SEQUENCE LISTING (30 sequences)

SEQ ID NO: 1 - DNA, 20 bp, Unknown
  Description of Unknown Nucleating polypeptide binding motif sequence
  modified_base (1)..(1): a, c, t or g
  modified_base (3)..(3): a, c, t or g
nbndccdshagrkgghrshv

SEQ ID NO: 2 - DNA, 20 bp, Unknown
  Description of Unknown Nucleating polypeptide binding motif sequence
  modified_base (18)..(18): a, c, t or g
  modified_base (20)..(20): a, c, t or g
vhsrhggkrgahsdccdnbn

SEQ ID NO: 3 - DNA, 7 bp, Artificial Sequence
  Description of Artificial Sequence Synthetic oligonucleotide
tatawaw

SEQ ID NO: 4 - DNA, 20 bp, Artificial Sequence
  Description of Artificial Sequence Synthetic oligonucleotide
  Description of Combined DNA/RNA Molecule Synthetic oligonucleotide
gacaggcagccatttuaaga

SEQ ID NO: 5 - DNA, 20 bp, Artificial Sequence
  Description of Artificial Sequence Synthetic oligonucleotide
  Description of Combined DNA/RNA Molecule Synthetic oligonucleotide
gacaggcagccatttuaaga

SEQ ID NO: 6 - RNA, 23 nt, Artificial Sequence
  Description of Artificial Sequence Synthetic oligonucleotide
uugacaggcagccauuuuaagag

SEQ ID NO: 7 - RNA, 23 nt, Artificial Sequence
  Description of Artificial Sequence Synthetic oligonucleotide
uugacaggcagccauuuuaagag

SEQ ID NO: 8 - RNA, 30 nt, Artificial Sequence
  Description of Artificial Sequence Synthetic oligonucleotide
cagacaggcagccauuuuaagagagaagag

SEQ ID NO: 9 - RNA, 30 nt, Artificial Sequence; identical to SEQ ID NO: 8
  Description of Artificial Sequence Synthetic oligonucleotide

SEQ ID NO: 10 - RNA, 30 nt, Artificial Sequence; identical to SEQ ID NO: 8
  Description of Artificial Sequence Synthetic oligonucleotide

SEQ ID NO: 11 - RNA, 30 nt, Artificial Sequence; identical to SEQ ID NO: 8
  Description of Artificial Sequence Synthetic oligonucleotide

SEQ ID NO: 12 - RNA, 117 nt, Artificial Sequence
  Description of Artificial Sequence Synthetic polynucleotide
guuggaaccauucaaaacagcauagcaaguuaaaauaaggcuaguccguuaucaacuugaaaaaguggcaccgagucggugcuuuuucucuucucucuuaaaauggcugccugucug

SEQ ID NO: 13 - RNA, 42 nt, Artificial Sequence
  Description of Artificial Sequence Synthetic oligonucleotide
gggugcggggcagagugugcguuuuagagcuaugcuguuuug

SEQ ID NO: 14 - RNA, 42 nt, Artificial Sequence
  Description of Artificial Sequence Synthetic oligonucleotide
ggugcggggcagagugugcgguuuuagagcuaugcuguuuug

SEQ ID NO: 15 - RNA, 42 nt, Artificial Sequence
  Description of Artificial Sequence Synthetic oligonucleotide
cggggcagagugugcggggaguuuuagagcuaugcuguuuug

SEQ ID NO: 16 - RNA, 42 nt, Artificial Sequence
  Description of Artificial Sequence Synthetic oligonucleotide
gggcgcugggggcucagguuguuuuagagcuaugcuguuuug

SEQ ID NO: 17 - RNA, 42 nt, Artificial Sequence
  Description of Artificial Sequence Synthetic oligonucleotide
aagggccuggccgggggugaguuuuagagcuaugcuguuuug

SEQ ID NO: 18 - RNA, 42 nt, Artificial Sequence
  Description of Artificial Sequence Synthetic oligonucleotide
ugagggggugagggggcgcuguuuuagagcuaugcuguuuug

SEQ ID NO: 19 - RNA, 42 nt, Artificial Sequence
  Description of Artificial Sequence Synthetic oligonucleotide
ggggcagagugugcggggaaguuuuagagcuaugcuguuuug

SEQ ID NO: 20 - RNA, 42 nt, Artificial Sequence
  Description of Artificial Sequence Synthetic oligonucleotide
gcggggaagggccuggccggguuuuagagcuaugcuguuuug

SEQ ID NO: 21 - RNA, 42 nt, Artificial Sequence
  Description of Artificial Sequence Synthetic oligonucleotide
ucacccccucacccccggccguuuuagagcuaugcuguuuug

SEQ ID NO: 22 - RNA, 42 nt, Artificial Sequence
  Description of Artificial Sequence Synthetic oligonucleotide
caagaccccuggaguucgaaguuuuagagcuaugcuguuuug

SEQ ID NO: 23 - protein, 4 aa, Artificial Sequence
  Description of Artificial Sequence Synthetic peptide
NANP

SEQ ID NO: 24 - DNA, 30 bp, Unknown
  Description of Unknown target ncRNA sequence
ctcttctctcttaaaatggctgcctgtctg

SEQ ID NO: 25 - protein, 7 aa, Artificial Sequence
  Description of Artificial Sequence Synthetic peptide
PKKKRKV

SEQ ID NO: 26 - DNA, 18 bp, Artificial Sequence
  Description of Artificial Sequence Synthetic primer
gggaatgagccgggagag

SEQ ID NO: 27 - DNA, 18 bp, Artificial Sequence
  Description of Artificial Sequence Synthetic primer
tgcgctgcgctcgctcct

SEQ ID NO: 28 - DNA, 20 bp, Artificial Sequence
  Description of Artificial Sequence Synthetic primer
tggactgaatcgttgacatg

SEQ ID NO: 29 - DNA, 18 bp, Artificial Sequence
  Description of Artificial Sequence Synthetic primer
ctttcccgttgccactgc

SEQ ID NO: 30 - RNA, 21 nt, Artificial Sequence
  Description of Artificial Sequence Synthetic oligonucleotide
cucuuaaaauggcugccuguc

(19) United States
(12) Patent Application Publication
(10) Pub. No.: US 2022/0403387 A1
(43) Pub. Date: Dec. 22, 2022

(54) MODULATING GENOMIC COMPLEXES

(71) Applicant: Flagship Pioneering Innovations V, Inc., Cambridge, MA (US)

(72) Inventors: Laura Gabriela Lande, Chestnut Hill, MA (US); David Arthur Berry, Newton, MA (US); Jodi Michelle Kennedy, Dedham, MA (US); Jeremiah Dale Farelli, Marblehead, MA (US)

(21) Appl. No.: 17/762,676

(22) PCT Filed: Sep. 22, 2020
(86) PCT No.: PCT/US20/51984

§ 371 Date: Mar. 22, 2022

Related U.S. Application Data
(60) Provisional application No. 62/904,437, filed on Sep. 23, 2019.

(51) Int. Cl. C12N 15/113

(52) U.S. Cl. CPC C12N 15/113; C12N 2310/113; C12N 2310/315; C12N 2310/3513; C12N 2310/321

(57) ABSTRACT

The present disclosure relates generally to modulation of genomic complexes via modulation of non-genomic components such as, non-coding RNAs.

### CROSS-REFERENCE TO RELATED APPLICATIONS

The present application claims priority to and benefit from U.S. provisional application U.S. Ser. No. 62/904,437 (filed Sep. 23, 2019), the contents of which is herein incorporated by reference.

### BACKGROUND

Modulation of certain genomic structures can impact gene expression. There is a need for novel tools to modulate genomic structures that influence expression, particularly in cases where over- or under-expression are associated with a disease (e.g., in a mammal, e.g., in humans).

### SUMMARY

The disclosure provides, among other things, modulating agents to alter genomic or transcription complexes, and/or the expression of one or more target genes, and methods of using and making the same. In some embodiments, a modulating agent comprises a targeting moiety and an effector moiety, wherein the targeting moiety comprises a nucleic acid that specifically binds to a non-coding RNA (ncRNA), e.g., an enhancer RNA (eRNA). In some embodiments, the ncRNA, e.g., eRNA, is part of a genomic or transcription complex associated with a target gene. Generally, binding of a targeting moiety to a ncRNA, e.g., eRNA, alters a property of a genomic or transcription complex, e.g., and consequently altering expression of a target gene. An effector moiety is a separate and different moiety than the targeting moiety, and in some embodiments either enhances the effect of the targeting moiety (e.g., on the genomic or transcription complex or expression) or provides an additional, e.g., different, effect beyond that provided by the targeting moiety.

Accordingly, in some aspects the disclosure is directed, in part, to a modulating agent, e.g, fusion molecule, comprising a targeting moiety that binds to a non-coding RNA (ncRNA), e.g., an enhancer RNA (eRNA), wherein the targeting moiety comprises a nucleic acid with a length of 10-50 nucleotides; and an effector moiety (e.g., covalently linked to the targeting moiety) that modulates, e.g., increases or decreases, expression of a gene regulated by the eRNA.

In another aspect, the disclosure is directed, in part, to a cell comprising a modulating agent, e.g., fusion molecule, described herein.

In another aspect, the disclosure is directed, in part, to a reaction mixture comprising a modulating agent, e.g., fusion molecule, described herein, and a cell.

In another aspect, the disclosure is directed, in part, to a pharmaceutical composition comprising a modulating agent, e.g., fusion molecule, described herein, and a pharmaceutically acceptable excipient.

In another aspect, the disclosure is directed, in part, to a method of treating a patient having aberrant expression of a target gene, the method comprising: administering to the patient a modulating agent, e.g., fusion molecule, described herein, thereby treating the patient having aberrant expression of a target gene.

In another aspect, the disclosure is directed, in part, to a method of decreasing expression of a target gene in a cell, the method comprising contacting the cell with a modulating agent, e.g., fusion molecule, described herein, thereby decreasing expression of the target gene.

In another aspect, the disclosure is directed, in part, to a method of modulating (e.g., inhibiting) a genomic complex or transcription complex, the method comprising contacting the genomic complex or transcription complex with a modulating agent, e.g., fusion molecule, described herein, wherein: (a) the level of genomic complex or transcription complex comprising the eRNA is altered (e.g., decreased) when the modulating agent, e.g., fusion molecule, is present relative to when it is absent; (b) the transcription complex comprises a transcription factor that binds the eRNA and the level of transcription complex comprising the transcription factor is altered (e.g., decreased) when the modulating agent, e.g., fusion molecule, is present relative to when it is absent; (c) the genomic complex comprises a genomic complex component that binds the eRNA and the level of genomic complex comprising the genomic complex component is altered (e.g., decreased) when the modulating agent, e.g., fusion molecule, is present relative to when it is absent; (d) the occupancy of the genomic complex or transcription complex at a target site is altered (e.g., decreased) when the modulating agent, e.g., fusion molecule, is present relative to when it is absent; or (e) the occupancy of the transcription factor or genomic complex component at a target site is altered (e.g., decreased) when the modulating agent, e.g., fusion molecule, is present relative to when it is absent.

In another aspect, the disclosure is directed, in part, to a method of delivering a modulating agent, e.g., fusion molecule, to a cell, e.g., a mammalian cell, comprising providing a modulating agent, e.g., fusion molecule, described herein; and contacting the cell with the modulating agent, e.g., fusion molecule, thereby delivering the modulating agent, e.g., fusion molecule, to the cell.

Additional features of any of the aforesaid methods or compositions include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Sep. 23, 2019. When one gene or protein references a plurality of sequence accession numbers, all of the sequence variants are encompassed.

### ENUMERATED EMBODIMENTS

1. A modulating agent, e.g, fusion molecule, comprising:
   a targeting moiety that binds to a non-coding RNA (ncRNA), e.g., an enhancer RNA (eRNA), wherein the targeting moiety comprises a nucleic acid with a length of 10-50 nucleotides, and
   an effector moiety that modulates, e.g., increases or decreases, expression of a gene regulated by the eRNA.
2. The modulating agent of embodiment 1, wherein the modulating agent is a fusion molecule.
3. A fusion molecule, comprising:
   a targeting moiety that binds to a non-coding RNA (ncRNA), e.g., an enhancer RNA (eRNA), wherein the targeting moiety comprises a nucleic acid with a length of 10-50 nucleotides, and
   an effector moiety covalently linked to the targeting moiety that modulates, e.g., increases or decreases, expression of a gene regulated by the eRNA.
4. The fusion molecule of either of embodiments 2 or 3, wherein the targeting moiety comprises no more than 50, 40, 30, or 20 nucleotides.
5. The fusion molecule of any of embodiments 2-4, wherein the fusion molecule comprises no more than 1300, 1200, 1100, 1000, 900, 800, 700, 600, 500, 400, 300, 200, or 100 amino acids.
6. The fusion molecule of any of embodiments 2-5, wherein the effector moiety does not comprise a CRISPR protein, e.g., does not comprise Cas9.
7. The fusion molecule of any of embodiments 2-6, wherein the effector moiety does not comprise CTCF or a functional fragment thereof.
8. The fusion molecule of any of embodiments 2-7, wherein the effector moiety does not bind to CTCF.
9. The fusion molecule of any of embodiments 2-8, wherein the effector moiety comprises an enzyme.
10. The fusion molecule of any of embodiments 2-9, wherein the effector moiety is capable of recruiting an endogenous protein, e.g., a protein endogenous to a cell, to the targeting moiety and/or eRNA.
11. The fusion molecule of any of embodiments 2-10, wherein the effector moiety comprises a genetic modifying moiety, e.g., chosen from a clustered regulatory interspaced short palindromic repeat (CRISPR) system, zinc finger nucleases (ZFNs), and Transcription Activator-Like Effector-based Nucleases (TALEN).
12. The fusion molecule of any of embodiments 2-11, wherein the effector moiety comprises an epigenetic modifying moiety, e.g., chosen from a DNA methylase (e.g., DNMT3a, DNMT3b, DNMTL); a DNA demethylation enzyme (e.g., members of the TET family); a histone methyltransferase; a histone deacetylase (e.g., HDAC1, HDAC2, HDAC3); sirtuin 1, 2, 3, 4, 5, 6, or 7; lysine-specific histone demethylase 1 (LSD1); histone-lysine-N-methyltransferase (Setdb1); euchromatic histone-lysine N-methyltransferase 2 (G9a); histone-lysine N-methyltransferase (SUV39H1); enhancer of zeste homolog 2 (EZH2); viral lysine methyltransferase (vSET); histone methyltransferase (SET2); or protein-lysine N-methyltransferase (SMYD2).
13. The fusion molecule of any of embodiments 2-12, wherein the effector moiety comprises a cleavable moiety, e.g., a moiety linked to the targeting moiety via a thrombin cleavable CPRSC linker.
14. The fusion molecule of any of embodiments 2-13, wherein the effector moiety comprises a small molecule.
15. The fusion molecule of any of embodiments 2-14, wherein the effector moiety comprises: methotrexate, NA bisulfite, or ammonium bisulfite.
16. The fusion molecule of any of embodiments 2-15, wherein the effector moiety comprises: a methyl transferase, a demethylase, a nuclease (e.g., Cas9), or a deaminase.
17. The fusion molecule of any of embodiments 2-16, wherein the effector moiety comprises: a peptide ligand, a full-length protein, a protein fragment, an antibody, an antibody fragment, a targeting aptamer, antigens, a receptors (e.g., glucagon-like peptide-1 (GLP-1), GLP-2 receptor 2, cholecystokinin B (CCKB), and somatostatin receptor), or a peptide therapeutic (e.g., those that bind to specific cell surface receptors such as G protein-coupled receptors (GPCRs) or ion channels; synthetic or analog peptides from naturally-bioactive peptides; anti-microbial peptides; pore-forming peptides; tumor targeting or cytotoxic peptides; or degradation or self-destruction peptides such as an apoptosis-inducing peptide signal or photosensitizer peptide).
18. The fusion molecule of any of embodiments 2-17, wherein the effector moiety comprises: a conjunction nucleating molecule, e.g., CTCF, cohesin, USF1, YY1, TATA-box binding protein associated factor 3 (TAF3), or ZNF143 binding motif.
19. The fusion molecule of any of embodiments 2-18, wherein the effector moiety comprises a DNA-binding domain.
20. The fusion molecule of any of embodiments 2-19, wherein the effector moiety comprises a gRNA, siRNA, RNAi molecule, or antisense oligonucleotide.
21. The fusion molecule of any of embodiments 2-20, wherein the effector moiety comprises Cas9.
22. The fusion molecule of any of embodiments 2-21, wherein the effector moiety comprises an aptamer, e.g., an oligonucleotide or peptide aptamer.
23. The fusion molecule of any of embodiments 2-22, wherein the targeting moiety comprises a protein or small molecule, e.g., an RNA-binding protein.
24. The fusion molecule of embodiment 23, wherein the targeting moiety comprises a Cas13 or Puf protein, or a functional fragment or variant thereof.
25. The fusion molecule of any of embodiments 2-24, wherein the targeting moiety comprises a gRNA, siRNA, or RNAi molecule.
26. The fusion molecule of any of embodiments 2-25, wherein the targeting moiety comprises a nucleic acid sequence that is complementary to the ncRNA, e.g., eRNA, or at least 80, 85, 90, 95, 99, or 100% identical to a sequence complementary to the ncRNA, e.g., eRNA.
27. The fusion molecule of any of embodiments 2-26, wherein the targeting moiety consists essentially of the nucleic acid with a length of 10-50 nucleotides.
28. The fusion molecule of any of embodiments 2-27, wherein the nucleic acid comprises one or more of deoxyribonucleic acids; ribonucleic acids, nucleic acid analogs (e.g., one or more of a peptide nucleic acid (PNA), a peptide-oligonucleotide conjugate, a locked nucleic acid (LNA), a bridged nucleic acid (BNA)); linkers; and combinations thereof.
29. The fusion molecule of any of embodiments 2-28, wherein the nucleic acid comprises one or more phosphorothioate bonds, e.g., between a pair of nucleic acids or nucleic acid analogs.
30. The fusion molecule of any of embodiments 2-29, wherein the K_{D} of the eRNA for another transcription factor, genomic complex component, or genomic sequence element increases by at least 1.05× (i.e., 1.05 times), 1.1×, 1.2×, 1.3×, 1.4×, 1.5×, 1.6×, 1.7×, 1.8×, 1.9×, 2×, 3×, 4×, 5×, 6×, 7×, 8×, 9×, 10×, 20×, 50×, or 100× (and optionally no more than 20×, 10×, 9×, 8×, 7×, 6×, 5×, 4×, 3×, 2×, 1.9×, 1.8×, 1.7×, 1.6×, 1.5×, 1.4×, 1.3×, 1.2×, or 1.1×) in the presence of the fusion molecule.
31. The fusion molecule of any of embodiments 2-30, wherein the level of a genomic complex or transcription complex comprising the eRNA decreases by at least 10, 20, 30, 40, 50, 60, 70, 80, 90, or 100% (and optionally, up to 100, 90, 80, 70, 60, 50, 40, 30, or 20%) in the presence of the fusion molecule.
32. The fusion molecule of any of embodiments 2-31, wherein binding of the targeting moiety to the eRNA decreases occupancy of a genomic complex or transcription complex comprising the eRNA at a genomic sequence element by at least 10, 20, 30, 40, 50, 60, 70, 80, 90, or 100% (and optionally, up to 100, 90, 80, 70, 60, 50, 40, 30, or 20%).
33. The fusion molecule of any of embodiments 2-32, wherein binding of the targeting moiety to the eRNA decreases occupancy of the eRNA in/at a genomic complex or transcription complex by at least 10, 20, 30, 40, 50, 60, 70, 80, 90, or 100% (and optionally, up to 100, 90, 80, 70, 60, 50, 40, 30, or 20%).
34. The fusion molecule of any of embodiments 2-33, wherein binding of the targeting moiety to the eRNA alters, e.g., decreases, the expression of a target gene, e.g., by at least 10, 20, 30, 40, 50, 60, 70, 80, 90, or 100% (and optionally, up to 100, 90, 80, 70, 60, 50, 40, 30, or 20%).
35. The fusion molecule of any of embodiments 2-34, wherein the nucleic acid comprises a sequence with at least 80, 85, 90, 95, 99, or 100% identity to SEQ ID NOs: 9013-9073, or having no more than 10, 9, 8, 7, 6, 5, 4, 3, 2, or 1 alterations (e.g., none) relative to a sequence selected from SEQ ID NOs: 9013-9073.
36. The fusion molecule of any of embodiments 2-35, wherein the fusion molecule comprises a peptide oligonucleotide conjugate.
37. The fusion molecule of any of embodiments 2-36, wherein the fusion molecule further comprises an additional moiety, e.g., a second effector or second targeting moiety, a tagging or monitoring moiety, membrane translocating moiety, pharmacoagent moiety, or a small molecule.
38. The fusion molecule of any of embodiments 2-37, wherein the fusion molecule further comprises a nuclear localization sequence (NLS).
39. The fusion molecule of any of embodiments 2-38, wherein the fusion molecule comprises a linker between the targeting moiety and the effector moiety.
40. A cell comprising the fusion molecule of any of embodiments 2-39.
41. A reaction mixture comprising the fusion molecule of any of embodiments 2-39 and a cell.
42. A pharmaceutical composition comprising the fusion molecule of any of embodiments 2-39 and a pharmaceutically acceptable excipient.
43. A method of treating a patient having aberrant expression of a target gene, the method comprising:
   administering to the patient the fusion molecule of any of embodiments 2-39,
   thereby treating the patient having aberrant expression of a target gene.
44. A method of decreasing expression of a target gene in a cell, the method comprising:
   contacting the cell with the fusion molecule of any of embodiments 2-39,
   thereby decreasing expression of the target gene.
45. The method or cell of any of embodiments 40, 43, or 44, wherein the cell comprises a genomic complex or transcription complex comprising the eRNA.
46. The method or cell of embodiment 45, wherein the level of genomic complex or transcription complex comprising the eRNA is altered (e.g., increased or decreased) when the fusion molecule is present relative to when it is absent.
47. The method or cell of embodiment 45, wherein the stability of the genomic complex or transcription complex at a target site is altered (e.g., increased or decreased) when the fusion molecule is present relative to when it is absent.
48. The method or cell of any of embodiments 45-47, wherein the cell comprises a genomic complex component or transcription factor that binds the eRNA.
49. The method or cell of embodiment 48, wherein the level of genomic complex component or transcription factor binding the eRNA is altered (e.g., increased or decreased) when the fusion molecule is present relative to when it is absent.
50. The method or cell of embodiment 48, wherein the stability of the transcription factor or genomic complex component at a target site is altered (e.g., increased or decreased) when the fusion molecule is present relative to when it is absent.
51. The method or cell of either of embodiments 47 or 50, wherein the target site is associated with expression of the target gene, e.g., and is selected from a promoter, enhancer, transcription start site, or coding sequence associated with the target gene.
52. A method of modulating (e.g., inhibiting or stabilizing) a genomic complex or transcription complex, the method comprising:
   contacting the genomic complex or transcription complex with the fusion molecule of any preceding claim,
   wherein:
   (a) the level of genomic complex or transcription complex comprising the eRNA is altered (e.g., increased or decreased) when the fusion molecule is present relative to when it is absent,
   (b) the transcription complex comprises a transcription factor that binds the eRNA and the level of transcription complex comprising the transcription factor is altered (e.g., increased or decreased) when the fusion molecule is present relative to when it is absent,
   (c) the genomic complex comprises a genomic complex component that binds the eRNA and the level of genomic complex comprising the genomic complex component is altered (e.g., increased or decreased) when the fusion molecule is present relative to when it is absent,
   (d) the occupancy of the genomic complex or transcription complex at a target site is altered (e.g., increased or decreased) when the fusion molecule is present relative to when it is absent, or
   (e) the occupancy of the transcription factor or genomic complex component at a target site is altered (e.g., increased or decreased) when the fusion molecule is present relative to when it is absent.
53. A method of delivering a fusion molecule to a cell, e.g., a mammalian cell, comprising:
   providing a fusion molecule of any of embodiments 2-39, and
   contacting the cell with the fusion molecule,
   thereby delivering the fusion molecule to the cell.
54. The fusion molecule, method, cell, or reaction mixture of any preceding claim, wherein the cell is selected from a neuronal cell (e.g., a CNS cell), a myocyte (e.g., a cardiomyocyte), a blood cell (e.g., an immune cell), an endothelial cell, a hepatocyte, a CD34+ cell, a CD3+ cell, or a fibroblast.

### Definitions

Anchor Sequence: The term “anchor sequence” as used herein, refers to a nucleic acid sequence recognized by a nucleating agent that binds sufficiently to form an anchor sequence-mediated conjunction, e.g., a complex. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction, or insulating against an enhancer that is outside the anchor sequence mediated conjunction. In some embodiments of the present disclosure, technologies are provided that may specifically target a particular anchor sequence or anchor sequences, without targeting other anchor sequences (e.g., sequences that may contain a nucleating agent (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule—e.g., the same chromosome) as the targeted anchor sequence is not modulated. In some embodiments, the anchor sequence comprises or is a nucleating polypeptide binding motif. In some embodiments, the anchor sequence is adjacent to a nucleating polypeptide binding motif.

Anchor Sequence-Mediated Conjunction: The term “anchor sequence-mediated conjunction” as used herein, refers to a DNA structure, in some cases, a complex, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequences (see, e.g. FIG. 1).

Associated with: Two events or entities are “associated” with one another, as that term is used herein, if presence, level, form and/or function of one is correlated with that of the other. For example, in some embodiments, a particular entity (e.g., polypeptide, genetic signature, metabolite, microbe, etc.) is considered to be associated with a particular disease, disorder, or condition, if its presence, level, form and/or function correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, a DNA sequence is “associated with” a target genomic or transcription complex when the nucleic acid is at least partially within the target genomic or transcription complex, and expression of a gene in the DNA sequence is affected by formation or disruption of the target genomic or transcription complex.

Domain: As used herein, the term “domain” refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, in some embodiments, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is or comprises a section or portion of a molecule (e.g., a small molecule, carbohydrate, lipid, nucleic acid, polypeptide, etc.). In some embodiments, a domain is or comprises a section of a polypeptide. In some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).

Effector moiety: As used herein, an “effector moiety” refers to a moiety (e.g., polypeptide) that alters a property of a genomic or transcription complex and/or modulates, e.g., inhibits, expression of a gene. In the context of a modulating agent, an effector moiety is a separate and different moiety than a targeting moiety also present in the modulating agent. In some embodiments, the effector moiety increases modulation of a gene targeted by the targeting moiety, compared to modulation of the same gene by the targeting moiety in the absence of the effector moiety, when the targeting moiety is in the nucleus. In some embodiments, the targeting moiety alone alters a property of a genomic or transcription complex and/or modulates, e.g., inhibits, gene expression, and the effector moiety increases that modulation of the gene expression. In some embodiments, an effector moiety is not a nuclear localization sequence (NLS). In some embodiments, a modulating agent comprises a targeting moiety, an effector moiety, and an NLS, wherein the effector moiety is not the same as the targeting moiety or NLS.

eRNA: As used herein, the term “eRNA” refers to an enhancer RNA. eRNA is a type of non-coding RNA that may be transcribed from an enhancer or portion of an enhancer. In some embodiments, an eRNA associates with, e.g., binds to, a genomic complex or transcription complex. An eRNA, in some embodiments, participates in transcription of one or more genes, e.g., genes regulated by that enhancer. In some embodiments, an eRNA is a component of a transcription complex or genomic complex, e.g., an anchor sequence-mediated conjunctions. In some embodiments, an eRNA is part of an anchor sequence-mediated conjunction that also comprises an enhancer (e.g., the enhancer encoding the eRNA) and a promoter operably linked to a target gene. In some embodiments, an eRNA is not part of an anchor sequence-mediated conjunction. In some embodiments, an eRNA may bind to one or more proteins, e.g., an anchor sequence nucleating protein such as CTCF and YY1, a component of the general transcription machinery, a protein known to be enriched in or near enhancers (e.g. Mediator, p300, etc.), or one or more transcriptional regulators (e.g., enhancer-binding proteins) (e.g., p53 or Oct4). In some embodiments, changes in levels of one or more eRNAs may correlate with and/or result in changes of levels of expression of a particular target gene. In some embodiments, for example, knockdown of an eRNA may correlate with and/or cause knockdown of a target gene.

Fusion Molecule: As used herein, the term “fusion molecule” refers to a compound comprising two or more moieties, e.g., a targeting moiety and an effector moiety, that alters a property of a genomic or transcription complex or modulates expression of a target gene, e.g., when present in the nucleus of a cell. In some embodiments, the two or more moieties, e.g., targeting moiety and effector moiety, are covalently-linked. A fusion molecule and its moieties may comprise any combination of polypeptide, nucleic acid, glycan, small molecule, or other components described herein (e.g., a targeting moiety may comprise a nucleic acid and an effector moiety may comprise a polypeptide). In some embodiments, a fusion molecule is a fusion protein, e.g., comprising one or more polypeptide domains covalently linked via peptide bonds. In some embodiments, a fusion molecule is a conjugate molecule that comprises a targeting moiety and effector moiety that are linked by a covalent bond other than a peptide bond or phosphodiester bond (e.g., a targeting moiety that comprises a nucleic acid and an effector moiety comprising a polypeptide linked by a covalent bond other than a peptide bond or phosphodiester bond). In some embodiments, a modulating agent is or comprises a fusion molecule.

Genomic complex: As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex binds. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory site (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., eRNA), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration (e.g., a loop) that it does not adopt when the complex is not formed.

Genomic Sequence Element: As used herein, the term “genomic sequence element” refers to a functional unit of nucleic acid situated in genomic DNA chosen from a gene, promoter, enhancer, anchor sequence, transcription factor binding site, a sequence proximal to any of the foregoing, or a portion of any of the foregoing. As used herein, proximal refers to a closeness of two sites, e.g., nucleic acid sites, such that binding of modulating agent at the first site and/or modification of the first site by modulating agent will produce the same or substantially the same effect as binding and/or modification of the other site. In some embodiments, proximal refers to a distance of less than 5000, 4000, 3000, 2000, 1000, 900, 800, 700, 600, 500, 400, 300, 200, 100, 50, or 25 base pairs.

Linker: As used herein, the term “linker” refers to a portion of a multi-element agent that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. In some embodiments, a linker consists essentially of amino acids; such a linker is referred to herein as a polypeptide linker. In some embodiments, a linker, e.g., polypeptide linker, is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length (and optionally no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length). In some embodiments, a linker tends not to adopt a rigid three-dimensional structure, but rather is structurally flexible. A variety of different linker elements that can appropriately be used when engineering polypeptides (e.g., fusion polypeptides) known in the art (see e.g., Holliger, P., et al. (1993) Proc. Natl. Acad. Sci. USA 90:6444-6448; Poljak, R. J., et al. (1994) Structure 2: 1 121-1123).

Non-natural amino acid: As used herein, the phrase “non-natural amino acid” refers to an entity having the chemical structure of an amino acid and therefore being capable of participating in at least two peptide bonds, but having an R group that differs from those found in nature. In some embodiments, non-natural amino acids may also have a second R group rather than a hydrogen, and/or may have one or more other substitutions on the amino or carboxylic acid moieties.

Peptide, Polypeptide, Protein: As used herein, the terms “peptide,” “polypeptide,” and “protein” refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types.

Target gene: As used herein, the term “target gene” means a gene that is targeted for modulation, e.g., of expression. In some embodiments, a target gene is part of a targeted genomic complex (e.g. a gene that has at least part of its genomic sequence as part of a target genomic complex, e.g. inside an anchor sequence-mediated conjunction), which genomic complex is targeted by one or more modulating agents as described herein. In some embodiments, modulation comprises inhibition of expression of the target gene. In some embodiments, a target gene is modulated by contacting the target gene or a genomic sequence element operably linked to the target gene with an active agent, e.g., fusion molecule, described herein. In some embodiments, a target gene is outside of a target genomic complex, for example, is a gene that encodes a component of a target genomic complex (e.g. a subunit of a transcription factor). In some embodiments, a target gene is aberrantly expressed (e.g., over-expressed) in a cell, e.g., a cell in a subject (e.g., patient).

Targeting moiety: As used herein, in the context of modulating agents, the term “targeting moiety” means an agent or entity that specifically binds with a component or set of components that participate in a genomic complex or transcription complex as described herein (e.g., in an anchor sequence-mediated conjunction). In some embodiments, a targeting moiety in accordance with the present disclosure targets one or more component(s) of a genomic complex as described herein. In some embodiments, a targeting moiety in accordance with the present disclosure targets one or more component(s) of a transcription complex as described herein. In some embodiments, a targeting moiety targets a genomic complex component other than a genomic sequence element. In some embodiments, a targeting moiety targets a plurality or combination of genomic complex components, which plurality in some embodiments may include a genomic sequence element. In some embodiments, a targeting moiety binds to an eRNA, e.g., an eRNA that is part of a genomic complex or transcription complex. In some aspects, contributions of the present disclosure include the insight that effective modulation of expression of a target gene and/or of a genomic complex or transcription complex, as described herein, can be achieved by targeting an eRNA that is part of a genomic complex or transcription complex comprising the target gene, e.g., using a fusion molecule comprising a targeting moiety and effector moiety. In some embodiments, the present disclosure contemplates that improved (e.g., with respect to, for example, degree of specificity for a particular genomic complex, transcription complex, or target gene) modulation may be achieved by targeting an eRNA that is part of a genomic complex or transcription complex associated with or comprising a target gene. Targeting a gene with a targeting moiety can comprise the targeting moiety binding to a ncRNA (e.g., an eRNA) that regulates expression of the gene.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as desired biological endpoint(s), substance to be delivered, target cell(s) or tissue(s), etc. For example, in some embodiments, an effective amount of compound in a formulation to treat a disease, disorder, and/or condition is an amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Transcription complex: As used herein, the term “transcription complex” is a complex that comprises at least one genomic sequence element, one or more transcription factor, and one or more non-coding RNAs (ncRNAs), e.g., an eRNA. In some embodiments, a genomic sequence element is chosen from a gene, an enhancer (e.g., associated with the gene), a promoter (e.g., associated with the gene), or an anchor sequence.

### BRIEF DESCRIPTION OF THE DRAWING

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1A is an illustration of an exemplary genomic complex as described herein.

FIG. 1B is an illustration of an exemplary genomic complex as described herein.

FIG. 1C is an illustration of an exemplary genomic complex as described herein.

FIG. 1D is an illustration of an exemplary genomic complex as described herein.

FIG. 1E is an illustration of an exemplary genomic complex as described herein.

FIG. 2 describes exemplary genomic complex modulating agents.

The patent or application file contains at least one drawing executed in color. Copies of this patent or patent application publication with color drawing(s) will be provided by the Office upon request and payment of the necessary fee.

### DETAILED DESCRIPTION OF CERTAIN EMBODIMENTS

The present disclosure provides technologies for modulating particular genomic or transcription complexes and/or altering, e.g., decreasing, expression of a target gene using a modulating agent, e.g., a fusion molecule, that targets an eRNA. In some embodiments, the eRNA is associated with, e.g., binds to, a genomic complex or transcription complex that comprises the target gene. By targeting the eRNA with a modulating agent, e.g., fusion molecule, disclosed herein, the level of associated genomic complex or transcription complex or the occupancy of the genomic complex or transcription complex at the target gene may be altered, e.g., decreased. In some embodiments, the eRNA is associated with, e.g., binds to, a genomic complex component or a transcription factor that associate with, e.g., bind to, a genomic complex or transcription complex comprising the target gene. In certain aspects, modulating agents, e.g., fusion molecules, that target an eRNA, and compositions comprising the same, are disclosed, as well as methods of characterizing, making, and/or using such agents are disclosed.

In some embodiments, provided compositions modulate transcription of one or more genes associated with a particular genomic complex or transcription complex (e.g., with a particular anchor sequence-mediated conjunction or genomic complex). The present disclosure teaches that compositions comprising a modulating agent, e.g., fusion molecule, as described herein that targets a genomic complex component or transcription factor, particularly eRNAs or genomic complex components or transcription factors comprising an eRNA, or combinations thereof, can modulate assembly and/or level of the particular genomic complex/transcription complex, and/or can modulate expression of one or more genes associated with (e.g., in operational proximity with) the particular genomic complex/transcription complex.

In some embodiments, a modulating agent, e.g., fusion molecule, is or comprises a targeting moiety that specifically targets the genomic complex component, transcription factor, eRNA, or combinations thereof.

In some aspects, contributions of the present disclosure include the insight that effective modulation of expression of a target gene and/or one or more genomic complexes can be achieved by targeting eRNA. In some embodiments, the present disclosure contemplates that improved (e.g., degree of specificity for a particular genomic complex or target gene as compared with other genomic complexes that may form or be formed in a given system or expression changes of other target genes) effectiveness of the modulation (e.g., in terms of impact on number of complexes detected in a population or relative expression change) may be achieved by targeting eRNA (e.g., eRNA that is part of a genomic complex or transcription complex comprising a target gene).

In some embodiments, the present disclosure provides technologies for altering particular genomic or transcription complexes (e.g., altering level of one or more particular genomic complexes) by targeting a non-genomic nucleic acid component of the complex. In some embodiments, a non-genomic nucleic acid suitable for targeting as described herein is an eRNA.

For example, those skilled in the art will be aware that certain genomic or transcription complexes may include one or more non-coding RNAs (ncRNAs) such as one or more enhancer RNAs (eRNAs). Those skilled in the art will be aware that eRNAs are typically transcribed from enhancers, and may participate in regulating expression of one or more genes regulated by the enhancer (target genes of the enhancer). In some embodiments, a genomic or transcription complex comprises an eRNA, an enhancer (e.g., the enhancer from which the eRNA was transcribed) and a promoter, e.g., operably linked to a target gene. In some embodiments, such a genomic or transcription complex further comprises one or more anchor sequence nucleating proteins such as CTCF and YY1, general transcription machinery components, Mediator, and/or one or more sequence-specific transcriptional regulatory agents such as p53 or Oct4. Without wishing to be bound by theory, changes in the level of an eRNA may result in changes of the level of expression of a target gene, e.g., by altering the level of genomic or transcription complex comprising the target gene or by altering the occupancy of the genomic or transcription complex at the target gene. In some embodiments, a modulating agent may target, e.g., bind, an eRNA, e.g., via a targeting moiety. In some embodiments, decreasing the level of an eRNA may cause a decrease in the level of expression of a target gene. By way of non-limiting example, knockdown of eRNAs listed in Table 1 (below) result in knockdown of particular target genes.

TABLE 1

| eRNA | Target |
|---|---|
| ncRNA-a1 | ECM1 |
| ncRNA-a2 | KLHL12 |
| ncRNA-a3 | TAL1 |
| ncRNA-a6 | Snai1 |
| ncRNA-a7 | Snai2 |
| TFF1-eRNA | TFF1 |
| FOXC1-eRNA | FOXC1 |
| CA12-eRNA | CA12 |
| PGR-eRNA | PGR |
| SIAH2-eRNA | SIAH2 |
| KCNK5-eRNA | KCNK5 |
| P2RY2-eRNA | P2RY2 |
| SMAD7-eRNA | SMAD7 |
| GREB1-eRNA | GREB1 |
| NRIP1-eRNA | NRIP1 |
| p53BER2 | PAPPA |
| p53BER4 | IER5 |

### Genomic Complexes

Genomic complexes relevant to the present disclosure include stable structures that comprise a plurality of polypeptide and/or nucleic acid components and that co-localize two or more genomic sequence elements (e.g., anchor sequences). Genomic complexes relevant to the present disclosure may include one or more eRNAs. In some embodiments, relevant genomic complexes are or comprise anchor sequence-mediated conjunctions.

### Anchor Sequence-Mediated Conjunction

In some embodiments, a genomic complex relevant to the present disclosure is or comprises an anchor sequence-mediated conjunction. In some embodiments, an anchor sequence-mediated conjunction is formed when nucleating protein(s) bind to anchor sequences in the genome and interactions between and among these proteins and, optionally, one or more other components, forms a conjunction in which the anchor sequences are physically co-localized. In some embodiments described herein, one or more genes is associated with an anchor sequence-mediated conjunction; in such embodiments, the anchor sequence-mediated conjunction typically includes one or more anchor sequences, one or more genes, and one or more transcriptional control sequences, such as an enhancing or silencing sequence. In some embodiments, a transcriptional control sequence is within, partially within, or outside an anchor sequence-mediated conjunction.

In some embodiments, an anchor sequence-mediated conjunction comprises or is associated with one or more genomic sequence elements. Genomic sequence elements involved in genomic complexes (e.g., an anchor sequence-mediated conjunction), may be non-contiguous with one another. In some embodiments with noncontiguous genomic sequence elements (e.g., anchor sequences), a first genomic sequence element (e.g., anchor sequence) may be separated from a second genomic sequence element (e.g., anchor sequence) by about 500 bp to about 500 Mb, about 750 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a first genomic sequence element (e.g., anchor sequence) is separated from a second genomic sequence element (e.g., anchor sequence) by about 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

In some embodiments, a genomic complex as described herein (e.g., an anchor sequence-mediated conjunction) is or comprises an intra-chromosomal complex. In certain embodiments, a genomic complex as described herein comprises a plurality of anchor sequence-mediated conjunctions. In some embodiments, a genomic complex (e.g., an anchor sequence-mediated conjunction) includes a TATA box, a CAAT box, a GC box, or a CAP site.

In some embodiments, an anchor sequence-mediated conjunction comprises a plurality of genomic complexes; in some such embodiments, an anchor sequence-mediated conjunction comprises at least one of an anchor sequence, a nucleic acid sequence, and a transcriptional control sequence in one or more genomic complexes.

In some aspects, compositions as provided herein may comprise a modulating agent that alters the level of a genomic complex and/or expression of a target gene. In some embodiments, the modulating agent comprise a targeting moiety that binds a component of a genomic complex, e.g., an eRNA, e.g., the presence of which can impact transcription of a gene associated with the genomic complex. In some embodiments, a modulating agent may modify one or more components of the targeted genomic complex, for example by physically interacting with one or more components of the complex, post-translationally modifying one or more components of the complex, and/or editing (e.g., by substitution, addition or deletion) one or more nucleic acid components of the complex.

In some embodiments, a genomic complex comprises one or more, e.g., 2, 3, 4, 5, or more, genes. In some embodiments, the present disclosure provides methods of modulating expression of a target gene in a complex comprising targeting a complex that achieves co-localization of genomic sequences that are outside of, not part of, or comprised within (i) a gene whose expression is modulated (e.g. a target gene); and/or (ii) one or more associated transcriptional control sequences that influence transcription of the gene whose expression is modulated.

In some embodiments, the present disclosure provides methods of modulating transcription of a target gene comprising targeting a complex that achieves co-localization of genomic sequences that are non-contiguous with (i) a gene whose expression is modulated; and/or (ii) associated transcriptional control sequences that influence transcription of the gene whose expression is modulated.

In some embodiments, an anchor sequence-mediated conjunction is associated with one or more, e.g., 2, 3, 4, 5, or more, transcriptional control sequences. In some embodiments, a target gene is non-contiguous with one or more transcriptional control sequences. In some embodiments where a gene is non-contiguous with its transcriptional control sequence(s), a gene may be separated from one or more transcriptional control sequences by about 100 bp to about 500 Mb, about 500 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a gene is separated from a transcriptional control sequence by about 100 bp, 300 bp, 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

In some embodiments, a particular type of anchor sequence-mediated conjunction (genomic complex) may help to determine how to modulate gene expression, e.g., choice of targeting moiety, by altering a genomic complex. For example, in some embodiments, some types of anchor sequence-mediated conjunctions comprise one or more transcription control sequences within an anchor sequence-mediated conjunction. Disruption of such a genomic complex by disrupting formation of a complex, e.g., altering one or more anchor sequences, is likely to decrease transcription of a target gene within a genomic complex.

In some embodiments, changes in structural features may alter post-nucleating activities and programs. In some embodiments, changes in structural features may result from changes to proteins, non-coding sequences, etc. that are part of a genomic complex but not part of a gene itself. In some embodiments, changes in non-structural (e.g., functional) features in absence of structural changes, may result from changes to proteins, non-coding sequences, etc.

### Anchor Sequences

In general, an anchor sequence is a genomic sequence element to which a genomic complex component binds specifically. In some embodiments, binding to an anchor sequence nucleates complex formation.

Each anchor sequence-mediated conjunction comprises one or more anchor sequences, e.g., a plurality. In some embodiments, anchor sequences can be manipulated or altered to disrupt naturally occurring complexes or to form one or more new complexes (e.g., to form exogenous complexes or to form non-naturally occurring complexes with exogenous or altered anchor sequences). Such alterations may modulate gene expression by, e.g., changing topological structure of DNA, e.g., by thereby modulating ability of a target gene to interact with gene regulation and control factors (e.g., enhancing and silencing/repressive sequences).

In some embodiments, chromatin structure is modified by substituting, adding or deleting one or more nucleotides within an anchor sequence-mediated conjunction. In some embodiments, chromatin structure is modified by substituting, adding, or deleting one or more nucleotides within an anchor sequence of an anchor sequence-mediated conjunction.

### Promoter Sequences

In some embodiments, a genomic complex as described herein achieves co-localization of genomic sequence elements that include a promoter. Those skilled in the art are aware that a promoter is, typically, a sequence element that initiates transcription of an associated gene. Promoters are typically near the 5′ end of a gene, not far from its transcription start site.

As those of ordinary skill are aware, transcription of protein-coding genes in eukaryotic cells is typically initiated by binding of general transcription factors (e.g., TFIID, TFIIE, TFIIH, etc.) and Mediator to core promoter sequences as a preinitiation complex that directs RNA polymerase II to the transcription start site, and in many instances remains bound to the core promoter sequences even after RNA polymerase escapes and elongation of the primary transcript is initiated.

In many embodiments, a promoter includes a sequence element such as TATA, Inr, DPE, or BRE, but those skilled in the art are well aware that such sequences are not necessarily required to define a promoter.

### Transcriptional Regulatory Sequences

In some embodiments, a genomic complex as described herein achieves co-localization of genomic sequence elements that include one or more transcriptional regulatory sequences. Those skilled in the art are familiar with a variety of positive (e.g., enhancers) or negative (e.g., repressors or silencers) transcriptional regulatory sequence elements that are associated with genes. Typically, when a cognate regulatory protein is bound to such a transcriptional regulatory sequence, transcription from the associated gene(s) is altered (e.g., increased for a positive regulatory sequence; decreased for a negative regulatory sequence.

### Detecting Genomic Complexes and Transcription Complexes

In some embodiments a given genomic complex or transcription complex is at a particular genomic site in a certain measurable quantity or configuration and administration of a modulating agent may change (e.g., decrease) an amount of complex present at a particular site. In some embodiments, alteration of a genomic complex or transcription complex by a modulating agent may change or impact another genomic complex or transcription complex located at a different genomic site.

In some embodiments, certain assays or tests may be conducted to determine presence or absence of one or more genomic or transcription complexes (e.g. presence or absence of one or more complexes in a given genomic location). In some embodiments, assays are conducted to determine if disruption of a genomic or transcription complex has been successful. In some embodiments, localization of complexes may be precisely performed via one or more assays. In some embodiments, assays are structural readouts. In some embodiments, assays are functional readouts. One of skill in the art, reading the present application, will have an understanding as to which assays and visualization techniques would be most appropriate to determine structure and/or function and/or activity (e.g. presence or absence) of genomic or transcription complexes.

In some embodiments, assays may quantify the amount of a particular genomic or transcription complex (e.g. Chromatin immunoprecipitation assays). In some embodiments, assays may visualize the presence of a particular modulating agent and/or genomic or transcription complex (e.g. immunostaining). In some embodiments, assays may both visualize and localize presence of a particular modulating agent and/or genomic or transcription complex (e.g. fluorescent in situ hybridization assays (FISH)). In some embodiments, a modulating agent will cause a detectable effect on function (e.g. functional assays in which an expected component of a genomic or transcription complex is changed in presence of a modulating agent, relative to absence of a modulating agent).

In some embodiments, an assay comprises a step of immunoprecipitation, e.g., chromatin immunoprecipitation.

In some embodiments, an assay comprises performing one or more serial chromatin immunoprecipitations, e.g., at least a first chromatin immunoprecipitation using an antibody against a first component of a targeted genomic or transcription complex, a second chromatin immunoprecipitation using an antibody against a second component of a targeted genomic or transcription complex, and optionally a step to determine presence and/or level of a genomic sequence that is in proximity to the genomic or transcription complex (e.g., a PCR assay).

In some embodiments, an assay is a chromosome conformation capture assay. In some embodiments, a chromosome capture assay detects presence and/or level of interactions between a single pair of genomic loci (e.g., a “one vs. one” assay, e.g., a 3C assay). In some embodiments, a chromosome capture assay detects presence and/or level of interactions between one genomic locus and multiple and/or all other genomic loci (e.g., a “one vs. many or all” assay, e.g., a 4C assay). In some embodiments, a chromosome capture assay detects presence and/or level of interactions between multiple and/or many genomic loci within a given region (e.g., a “many vs. many” assay, e.g., a 5C assay). In some embodiments, a chromosome capture assay detects presence and/or level of interactions between all or nearly all genomic loci (e.g., an “all vs. all” assay, e.g., a Hi-C assay).

In some embodiments, an assay comprises a step of cross-linking cell genomes (e.g., using formaldehyde). In some embodiments, an assay comprises a capture step (e.g., using an oligonucleotide) to enrich for specific loci or for a specific locus of interest. In some embodiments, an assay is a single-cell assay.

In some embodiments, an assay detects interactions between genomic loci at a genome-wide level, e.g., a Chromatin Interaction Analysis by Paired-End Tag Sequencing (ChiA-PET) assay. All references and publications cited herein are hereby incorporated by reference.

### Transcription Complexes

Transcription complexes relevant to the present disclosure include structures that comprise at least one genomic sequence element (e.g., a gene, promoter, or transcriptional control sequence, e.g., enhancer, silencer, or repressor), one or more transcription factor, and one or more ncRNA, e.g., an eRNA. In some embodiments, a transcription complex may be a genomic complex. In some embodiments, a genomic complex may be or comprise a transcription complex. For example, a transcription complex may comprise a gene, a promoter operably linked to the gene, a transcription factor (e.g., bound to the promoter), an enhancer, and an eRNA (e.g., which was transcribed from the enhancer). If the transcription complex also co-localized two or more genomic sequence elements (e.g., anchor sequences), it would also be a genomic complex.

In some embodiments, the present disclosure provides technologies for altering particular transcription complexes (e.g., altering level of one or more particular transcription complexes) by targeting a non-genomic nucleic acid component of the complex. In some embodiments, a non-genomic nucleic acid suitable for targeting as described herein is an eRNA.

For example, those skilled in the art will be aware that certain transcription complexes may include one or more non-coding RNAs (ncRNAs) such as one or more enhancer RNAs (eRNAs). Those skilled in the art will be aware that eRNAs are typically transcribed from enhancers, and may participate in regulating expression of one or more genes regulated by the enhancer (target genes of the enhancer). In some embodiments, a transcription complex comprises an eRNA, an enhancer (e.g., the enhancer from which the eRNA was transcribed) and a promoter, e.g., operably linked to a target gene. In some embodiments, such a transcription complex further comprises one or more anchor sequence nucleating proteins such as CTCF and YY1. A transcription complex comprises one or more transcription factors. One of skill in the art will appreciate that transcription factors include sequence specific factors (e.g., that promote transcription of a particular gene or genes, e.g., p53 or Oct4) and general transcription machinery components, e.g., Mediator. Without wishing to be bound by theory, changes in the level of an eRNA may result in changes of the level of expression of a target gene, e.g., by altering the level of transcription complex comprising the target gene or by altering the occupancy of the transcription complex at the target gene. In some embodiments, a modulating agent may target, e.g., bind, an eRNA, e.g., via a targeting moiety. In some embodiments, decreasing the level of an eRNA may cause a decrease in the level of a target gene. By way of non-limiting example, knockdown of eRNAs listed in Table 1 (below) result in knockdown of particular target genes.

### Modulating Agents

As described herein, the present disclosure provides technologies for modulating, e.g., disrupting, genomic complexes and/or transcription complexes by contacting a system in which such complexes have formed or would otherwise be expected to form with a modulating agent as described herein. In some embodiments, the extent of complex formation and/or maintenance (e.g., number of complexes in a system at a given moment in time, or over a period of time) is altered (e.g., reduced) by the presence of the modulating agent as compared with the extent observed in the absence of the modulating agent. In general, a modulating agent as described herein interacts with one or more enhancer RNAs (eRNAs). In some embodiments, modulating agents do not target genomic sequence elements. In some embodiments, targeting may include targeting of one or more genomic sequence elements, for example, in addition to targeting one or more eRNAs.

In some embodiments, a modulating agent disrupts one or more aspects of a complex (e.g., a genomic complex or transcription complex whose component(s) is/are targeted). In some embodiments, disruption is or comprises disruption of a topological structure of a genomic complex. In some embodiments, disruption of a topological structure of a genomic complex results in altered, e.g., decreased, expression of a given target gene. In some embodiments, no detectable disruption of a topological structure is observed, but altered expression of a given target gene is nonetheless observed. In some embodiments, disruption is or comprises binding to a component, e.g, an eRNA, of a complex (e.g., genomic or transcription complex). Binding may result in sequestering of the component, e.g., eRNA, or degradation of the component, e.g., eRNA (e.g., by an enzyme of the cell); in either exemplary case, the level of the component, e.g., eRNA, is altered, e.g., decreased, and the level or occupancy of the genomic/transcription complex, e.g., at a target gene, is thereby altered.

In some embodiments, a modulating agent strengthens or promotes one or more aspects of a complex (e.g., a genomic complex or transcription complex whose component(s) is/are targeted). In some embodiments, strengthening or promoting comprises stabilization of a topological structure of a genomic complex. In some embodiments, strengthening or promoting of a topological structure of a genomic complex results in altered, e.g., decreased or increased, expression of a given target gene. In some embodiments, no detectable alteration of a topological structure is observed, but altered expression of a given target gene is nonetheless observed. In some embodiments, strengthening or promoting is or comprises binding to a component, e.g, an eRNA, of a complex (e.g., genomic or transcription complex). Binding may result in stabilization of an interaction of the component, e.g., eRNA, with another genomic complex component or transcription factor, e.g., and thereby the level or occupancy of the genomic/transcription complex, e.g., at a target gene, is altered.

In some embodiments, contacting a target component, e.g., eRNA, of a genomic/transcription complex with a modulating agent results in alteration of gene expression. In some embodiments, alteration may be or comprise a change (e.g. decrease in expression) relative to gene expression in the absence of a modulating agent.

A modulating agent may bind its target component, e.g., eRNA, of a genomic/transcription complex and alter formation of the genomic/transcription complex (e.g., by altering affinity of the targeted component to one or more other complex components, e.g., by at least 10%, 15%, 20%, 25%, 30%, 35%, 40%, 45%, 50%, 55%, 60%, 65%, 70%, 75%, 80%, 85%, 90%, 95%, or more). Alternatively or additionally, in some embodiments, binding by a modulating agent alters topology of genomic DNA impacted by a genomic complex, e.g., at least 10%, 15%, 20%, 25%, 30%, 35%, 40%, 45%, 50%, 55%, 60%, 65%, 70%, 75%, 80%, 85%, 90%, 95%, or more). In some embodiments, a modulating agent alters expression of a gene associated with a targeted genomic/transcription complex by at least 10%, 15%, 20%, 25%, 30%, 35%, 40%, 45%, 50%, 55%, 60%, 65%, 70%, 75%, 80%, 85%, 90%, 95%, or more. In some embodiments, formation of a genomic/transcription complex and/or changes to genomic DNA topology are assessed by ChIP-Seq, ChIA-PET, RNA-Seq, and/or qPCR.

In some embodiments, a modulating agent disrupts a genomic or transcription complex by targeting an eRNA. In some embodiments, a modulating agent physically interferes with formation and/or maintenance of a genomic complex. In some embodiments, a modulating agent binds to an eRNA to disrupt a genomic complex.

In some embodiments, the present disclosure provides a modulating agent, comprising a targeting moiety that binds specifically to one or more eRNAs, and/or to a genomic complex component or transcription factor that would otherwise bind to said eRNA(s), and not to non-targeted sequences, e.g., non-targeted eRNAs, (or genomic complex components or transcription factors that bind to them). In some embodiments, said binding of targeting moiety to eRNA occurs within a cell, e.g., with sufficient affinity that it competes for binding of an endogenous polypeptide (e.g., a genomic complex component or transcription factor which binds the eRNA) within a cell.

In some embodiments, the present disclosure provides a modulating agent comprising an effector moiety which enhances the modulation of the expression, e.g., decrease of expression, of a target gene in addition to or separate from any effect a targeting moiety may have on expression of the target gene. In some embodiments, the effector moiety decreases expression of the target gene. In some embodiments, the effector moiety does not bind to an eRNA (e.g., the eRNA which the targeting moiety binds to).

As described in more detail below, a modulating agent (and/or any of a targeting moiety, effector moiety, and/or other moiety) may be or comprise a polypeptide, e.g., a protein or protein fragment, an antibody or antibody fragment (e.g., an antigen-binding fragment, a fusion molecule, etc), an oligonucleotide, a peptide nucleic acid, a small molecule, etc. and/or may include one or more non-natural residues or other structures. In some embodiments, a modulating agent may be or include an aptamer and/or a pharmacoagent, particularly one with poor pharmacokinetics as described herein.

A modulating agent may be or comprise a fusion molecule. In some embodiments, a fusion molecule comprises a targeting moiety and an effector moiety which are covalently connected to one another.

In some embodiments, a fusion molecule, e.g., the targeting moiety of a fusion molecule, comprises no more than 100, 90, 80, 70, 60, 50, 40, 30, or 20 nucleotides (and optionally at least 10, 20, 30, 40, 50, 60, 70, 80, or 90 nucleotides). In some embodiments, a fusion molecule, e.g., the effector moiety of a fusion molecule, comprises no more than 2000, 1900, 1800, 1700, 1600, 1500, 1400, 1300, 1200, 1100, 1000, 900, 800, 700, 600, 500, 400, 300, 200, or 100 amino acids (and optionally at least 50, 100, 200, 300, 400, 500, 600, 700, 800, 900, 1000, 1100, 1200, 1300, 1400, 1500, 1600, 1700, 1800, or 1900 amino acids). In some embodiments, a fusion molecule, e.g., the effector moiety of a fusion molecule, comprises 100-2000, 100-1900, 100-1800, 100-1700, 100-1600, 100-1500, 100-1400, 100-1300, 100-1200, 100-1100, 100-1000, 100-900, 100-800, 100-700, 100-600, 100-500, 100-400, 100-300, 100-200, 200-2000, 200-1900, 200-1800, 200-1700, 200-1600, 200-1500, 200-1400, 200-1300, 200-1200, 200-1100, 200-1000, 200-900, 200-800, 200-700, 200-600, 200-500, 200-400, 200-300, 300-2000, 300-1900, 300-1800, 300-1700, 300-1600, 300-1500, 300-1400, 300-1300, 300-1200, 300-1100, 300-1000, 300-900, 300-800, 300-700, 300-600, 300-500, 300-400, 400-2000, 400-1900, 400-1800, 400-1700, 400-1600, 400-1500, 400-1400, 400-1300, 400-1200, 400-1100, 400-1000, 400-900, 400-800, 400-700, 400-600, 400-500, 500-2000, 500-1900, 500-1800, 500-1700, 500-1600, 500-1500, 500-1400, 500-1300, 500-1200, 500-1100, 500-1000, 500-900, 500-800, 500-700, 500-600, 600-2000, 600-1900, 600-1800, 600-1700, 600-1600, 600-1500, 600-1400, 600-1300, 600-1200, 600-1100, 600-1000, 600-900, 600-800, 600-700, 700-2000, 700-1900, 700-1800, 700-1700, 700-1600, 700-1500, 700-1400, 700-1300, 700-1200, 700-1100, 700-1000, 700-900, 700-800, 800-2000, 800-1900, 800-1800, 800-1700, 800-1600, 800-1500, 800-1400, 800-1300, 800-1200, 800-1100, 800-1000, 800-900, 900-2000, 900-1900, 900-1800, 900-1700, 900-1600, 900-1500, 900-1400, 900-1300, 900-1200, 900-1100, 900-1000, 1000-2000, 1000-1900, 1000-1800, 1000-1700, 1000-1600, 1000-1500, 1000-1400, 1000-1300, 1000-1200, 1000-1100, 1100-2000, 1100-1900, 1100-1800, 1100-1700, 1100-1600, 1100-1500, 1100-1400, 1100-1300, 1100-1200, 1200-2000, 1200-1900, 1200-1800, 1200-1700, 1200-1600, 1200-1500, 1200-1400, 1200-1300, 1300-2000, 1300-1900, 1300-1800, 1300-1700, 1300-1600, 1300-1500, 1300-1400, 1400-2000, 1400-1900, 1400-1800, 1400-1700, 1400-1600, 1400-1500, 1500-2000, 1500-1900, 1500-1800, 1500-1700, 1500-1600, 1600-2000, 1600-1900, 1600-1800, 1600-1700, 1700-2000, 1700-1900, 1700-1800, 1800-2000, 1800-1900, or 1900-2000 amino acids.

A modulating agent, e.g., fusion molecule, may comprise a polypeptide, e.g., an RNA-binding protein, e.g., an RNA-binding protein that targets a ncRNA, e.g., eRNA. In some embodiments, an RNA-binding protein is chosen from Puf, Cas13, or a variant or functional fragment of either thereof. In some embodiments, a targeting moiety comprises a polypeptide, e.g., an RNA-binding protein, e.g., Puf or Cas13. In some embodiments, an effector moiety comprises a polypeptide, e.g., an RNA-binding protein, e.g., Puf or Cas13. In some embodiments, a modulating agent, e.g., fusion molecule, comprises a targeting moiety comprising an RNA-binding protein, and an effector moiety that strengthens, promotes, or stabilizes a genomic complex or transcription complex. In some embodiments, the effector moiety is or comprises p300, VP16, VP64, VP160, or a functional fragment or variant of any thereof.

A modulating agent, e.g., fusion molecule, may comprise nucleic acid, e.g., one or more nucleic acids. The term “nucleic acid” refers to any compound that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

In some embodiments, a targeting moiety comprises or is nucleic acid. In some embodiments, an effector moiety comprises or is nucleic acid. In some embodiments, a nucleic acid that may be included in a nucleic acid moiety or entity as described herein, may be or comprise DNA, RNA, and/or an artificial or synthetic nucleic acid or nucleic acid analog or mimic. For example, in some embodiments, a nucleic acid included in a nucleic acid moiety as described herein may be or include one or more of genomic DNA (gDNA), complementary DNA (cDNA), a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA as described herein and/or that targets an expression product of a particular gene associated with a targeted genomic complex as described herein), etc. In some embodiments, a nucleic acid may include one or more residues that is not a naturally-occurring DNA or RNA residue, may include one or more linkages that is/are not phosphodiester bonds (e.g., that may be, for example, phosphorothioate bonds, etc), and/or may include one or more modifications such as, for example, a 2′O modification such as 2′-OMeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

In some embodiments, nucleic acids may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

Some examples of nucleic acids include, but are not limited to, a nucleic acid that hybridizes to an endogenous gene (e.g., gRNA or antisense ssDNA as described herein elsewhere), a nucleic acid that hybridizes to an exogenous nucleic acid such as a viral DNA or RNA, nucleic acid that hybridizes to an RNA, a nucleic acid that interferes with gene transcription, a nucleic acid that interferes with RNA translation, a nucleic acid that stabilizes RNA or destabilizes RNA such as through targeting for degradation, a nucleic acid that interferes with a DNA or RNA binding factor through interference of its expression or its function, a nucleic acid that is linked to a intracellular protein or protein complex and modulates its function, etc.

The present disclosure contemplates modulating agents comprising RNA therapeutics (e.g., modified RNAs) as useful components of provided compositions as described herein. For example, in some embodiments, a modified mRNA encoding a protein of interest may be linked to a polypeptide described herein and expressed in vivo in a subject.

In some embodiments, a modulating agent, e.g., fusion molecule, comprises one or more nucleoside analogs. In some embodiments, a nucleic acid sequence may include in addition or as an alternative to one or more natural nucleosides nucleosides, e.g., purines or pyrimidines, e.g., adenine, cytosine, guanine, thymine and uracil, one or more nucleoside analogs. In some embodiments, a nucleic acid sequence includes one or more nucleoside analogs. A nucleoside analog may include, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-β]pyridine, and any others that can base pair with a purine or a pyrimidine side chain.

In some embodiments, a modulating agent, e.g., fusion molecule, comprises a nucleic acid sequence that encodes a gene expression product.

In some embodiments, a targeting moiety comprises a nucleic acid that does not encode a gene expression product. For example, a targeting moiety may comprise an oligonucleotide that hybridizes to a ncRNA, e.g., an eRNA. For example, in some embodiments, a sequence of an oligonucleotide comprises a complement of a target eRNA, or has a sequence that is at least 80%, at least 85%, at least 90%, at least 95%, at least 97%, at least 98%, at least 99% identical to the complement of a target eRNA.

A nucleic acid sequence suitable for use in a modulating agent, e.g., fusion molecule, may include, but is not limited to, DNA, RNA, modified oligonucleotides (e.g., chemical modifications, such as modifications that alter backbone linkages, sugar molecules, and/or nucleic acid bases), and artificial nucleic acids. In some embodiments, a nucleic acid sequence includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules.

In some embodiments, a nucleic acid sequence suitable for use in a modulating agent, e.g., fusion molecule, has a length from about 15-200, 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 215-190, 20-190, 30-190, 40-190, 50-190, 60-190, 70-190, 80-190, 90-190, 100-190, 110-190, 120-190, 130-190, 140-190, 150-190, 160-190, 170-190, 180-190, 15-180, 20-180, 30-180, 40-180, 50-180, 60-180, 70-180, 80-180, 90-180, 100-180, 110-180, 120-180, 130-180, 140-180, 150-180, 160-180, 170-180, 15-170, 20-170, 30-170, 40-170, 50-170, 60-170, 70-170, 80-170, 90-170, 100-170, 110-170, 120-170, 130-170, 140-170, 150-170, 160-170, 15-160, 20-160, 30-160, 40-160, 50-160, 60-160, 70-160, 80-160, 90-160, 100-160, 110-160, 120-160, 130-160, 140-160, 150-160, 215-150, 20-150, 30-150, 40-150, 50-150, 60-150, 70-150, 80-150, 90-150, 100-150, 110-150, 120-150, 130-150, 140-150, 15-140, 20-140, 30-140, 40-140, 50-140, 60-140, 70-140, 80-140, 90-140, 100-140, 110-140, 120-140, 130-140, 15-130, 20-130, 30-130, 40-130, 50-130, 60-130, 70-130, 80-130, 90-130, 100-130, 110-130, 120-130, 215-120, 20-120, 30-120, 40-120, 50-120, 60-120, 70-120, 80-120, 90-120, 100-120, 110-120, 15-110, 20-110, 30-110, 40-110, 50-110, 60-110, 70-110, 80-110, 90-110, 100-110, 15-100, 20-100, 30-100, 40-100, 50-100, 60-100, 70-100, 80-100, 90-100, 15-90, 20-90, 30-90, 40-90, 50-90, 60-90, 70-90, 80-90, 15-80, 20-80, 30-80, 40-80, 50-80, 60-80, 70-80, 15-70, 20-70, 30-70, 40-70, 50-70, 60-70, 15-60, 20-60, 30-60, 40-60, 50-60, 15-50, 20-50, 30-50, 40-50, 15-40, 20-40, 30-40, 15-30, 20-30, or 15-20 nucleotides, or any range therebetween.

In some embodiments, a nucleic acid (e.g., a nucleic acid encoding a modulating agent, e.g., fusion molecule, or a nucleic acid that is comprised in a modulating agent, e.g., fusion molecule) may comprise operably linked sequences. The term “operably linked” describes a relationship between a first nucleic acid sequence and a second nucleic acid sequence wherein the first nucleic acid sequence can affect the second nucleic acid sequence, e.g., by being co-expressed together, e.g., as a fusion gene, and/or by affecting transcription, epigenetic modification, and/or chromosomal topology. In some embodiments, operably linked means two nucleic acid sequences are comprised on the same nucleic acid molecule. In a further embodiment, operably linked may further mean that the two nucleic acid sequences are proximal to one another on the same nucleic acid molecule, e.g., within 1000, 500, 100, 50, or 10 base pairs of each other or directly adjacent to each other. In an embodiment, a promoter or enhancer sequence that is operably linked to a sequence encoding a protein can promote the transcription of the sequence encoding a protein, e.g., in a cell or cell free system capable of performing transcription. In an embodiment, a first nucleic acid sequence encoding a protein or fragment of a protein that is operably linked to a second nucleic acid sequence encoding a second protein or second fragment of a protein are expressed together, e.g., the first and second nucleic acid sequences comprise a fusion gene and are transcribed and translated together to produce a fusion protein.

### Targeting Moiety

In some embodiments, a modulating agent is or comprises a fusion molecule comprising a targeting moiety. In some embodiments, a targeting moiety targets, e.g., binds, an eRNA, e.g., an eRNA that is a component of a genomic complex or transcription complex, or to a genomic complex component or transcription factor that binds an eRNA. The target of a targeting moiety may be referred to as its targeted component (e.g., an eRNA, or a genomic complex component or transcription factor which binds the eRNA).

In some embodiments, interaction between a targeting moiety and its targeted component interferes with one or more other interactions that the targeted component would otherwise make. In some embodiments, binding of a targeting moiety to a targeted component prevents the targeted component from interacting with another transcription factor, genomic complex component, or genomic sequence element. In some embodiments, binding of a targeting moiety to a targeted component decreases binding affinity of the targeted component for another transcription factor, genomic complex component, or genomic sequence element. In some embodiments, K_{D} of a targeted component for another transcription factor, genomic complex component, or genomic sequence element increases by at least 1.05× (i.e., 1.05 times), 1.1×, 1.2×, 1.3×, 1.4×, 1.5×, 1.6×, 1.7×, 1.8×, 1.9×, 2×, 3×, 4×, 5×, 6×, 7×, 8×, 9×, 10×, 20×, 50×, or 100× (and optionally no more than 20×, 10×, 9×, 8×, 7×, 6×, 5×, 4×, 3×, 2×, 1.9×, 1.8×, 1.7×, 1.6×, 1.5×, 1.4×, 1.3×, 1.2×, or 1.1×) in presence of a modulating agent, e.g., fusion molecule, comprising the targeting moiety than in the absence of the modulating agent, e.g., fusion molecule, comprising the targeting moiety. In some embodiments, the binding affinity and/or K_{D} are determined using ChIP-Seq.

In some embodiments, binding of a targeting moiety to a targeted component alters, e.g., decreases, the level of a genomic complex or transcription complex comprising the targeted component. In some embodiments, the level of a genomic complex or transcription complex comprising the targeted component decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., fusion molecule, comprising the targeting moiety relative to the absence of said modulating agent. In some embodiments, binding of a targeting moiety to a targeted component alters, e.g., decreases, occupancy of the genomic complex or transcription complex at a genomic sequence element (e.g., a target gene, or an enhancer associated with a targeted eRNA). In some embodiments, occupancy decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., fusion molecule, comprising the targeting moiety relative to the absence of said modulating agent. In some embodiments, formation of a genomic/transcription complex and/or changes to genomic DNA topology are assessed by ChIP-Seq, ChIA-PET, RNA-Seq, and/or qPCR.

In some embodiments, binding of a targeting moiety to a targeted component alters, e.g., decreases the occupancy of the genomic complex or transcription complex at a genomic sequence element (e.g., a gene, promoter, or enhancer, e.g., associated with the geonmic or transcription complex). In some embodiments, binding of a targeting moiety to a targeted component decreases occupancy of the genomic complex or transcription complex at a genomic sequence element by at least 10, 20, 30, 40, 50, 60, 70, 80, 90, or 100% (and optionally, up to 100, 90, 80, 70, 60, 50, 40, 30, or 20%) in the presence of a modulating agent, e.g., fusion molecule, comprising the targeting moiety relative to the absence of said modulating agent. In some embodiments, occupancy refers to the frequency with which an element can be found associated with another element, e.g., as determined by HiC, ChIP, immunoprecipitation, or other association measuring assays known in the art.

In some embodiments, binding of a targeting moiety to a targeted component alters, e.g., decreases the occupancy of the targeted component in/at the genomic complex or transcription complex. In some embodiments, binding of a targeting moiety to a targeted component decreases occupancy of the targeted component in/at the genomic complex or transcription complex by at least 10, 20, 30, 40, 50, 60, 70, 80, 90, or 100% (and optionally, up to 100, 90, 80, 70, 60, 50, 40, 30, or 20%) in the presence of a modulating agent, e.g., fusion molecule, comprising the targeting moiety relative to the absence of said modulating agent.

In some embodiments, binding of a targeting moiety to a targeted component alters, e.g., decreases, the expression of a target gene associated with the genomic complex or transcription complex comprising the targeted component. In some embodiments, the expression of the target gene decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., fusion molecule, comprising the targeting moiety relative to the absence of said modulating agent.

In some embodiments, a target gene is “associated with” a genomic or transcription complex, e.g., an anchor sequence-mediated conjunction, if formation or disruption of the genomic or transcription complex, e.g., anchor sequence-mediated conjunction causes an alteration in expression (e.g., transcription) of the target gene. For example, in some embodiments, formation or disruption of an anchor sequence-mediated conjunction causes an enhancing or silencing/repressive sequence to associate with or become unassociated with a target gene.

In some embodiments, a targeting moiety is or comprises a nucleic acid (e.g., an oligonucleotide (e.g., a gRNA, siRNA, etc.) which, in some embodiments, may contain one or more modified residues, linkages, or other features), a polypeptide (e.g., a protein, a protein fragment, an antibody, an antibody fragment (e.g., an antigen-binding fragment), or both. In some embodiments, the targeting moiety may include one or more modified residues, linkages, or other features), peptide nucleic acid, small molecule, etc.

Those skilled in the art reading the present disclosure will appreciate that, in some embodiments, a modulating agent is complex-specific. That is, in some embodiments, a targeting moiety binds specifically to its targeted component in one or more genomic or transcription complexes (e.g., within a cell) and not to non-targeted genomic or transcription complexes (e.g., within the same cell). In some embodiments, a modulating agent specifically targets a genomic complex that is present in only certain cell types and/or present at certain developmental stages or times.

In some embodiments, a targeting moiety is designed and/or administered so that it specifically interacts with a particular genomic or transcription complex relative to other genomic or transcription complexes that may be present in the same system (e.g., cell, tissue, etc).

In some embodiments, a targeting moiety comprises a nucleic acid sequence complementary to a targeted component, e.g., an eRNA, in a genomic or transcription complex. In some embodiments, a targeting moiety comprises a nucleic acid sequence that is complementary to at least 10%, 15%, 20%, 25%, 30%, 35%, 40%, 45%, 50%, 55%, 60%, 65%, 70%, 75%, 80%, 85%, 90%, 95%, or more of a targeted component, e.g., eRNA, in a genomic or transcription complex.

In some embodiments, a targeting moiety comprises a nucleic acid sequence that is at least 50, 60, 70, 75, 80, 85, 90, 91, 92, 93, 94, 95, 96, 97, 98, 99, or 100% identical to a sequence selected from SEQ ID NOs: 9013-9073. In some embodiments, a targeting moiety comprises a nucleic acid sequence that comprises no more than 1, 2, 3, 4, 5, 6, 7, 8, 9, 10, 11, 12, 13, 14, or 15 alterations (e.g., substitutions, deletions, or insertions) relative to a sequence selected from SEQ ID NOs: 9013-9073.

In some embodiments, a targeting moiety comprises a nucleic acid sequence that is at least 50, 60, 70, 75, 80, 85, 90, 91, 92, 93, 94, 95, 96, 97, 98, 99, or 100% identical to the complement of a sequence selected from SEQ ID NOs: 9013-9073. In some embodiments, a targeting moiety comprises a nucleic acid sequence that comprises no more than 1, 2, 3, 4, 5, 6, 7, 8, 9, 10, 11, 12, 13, 14, or 15 alterations (e.g., substitutions, deletions, or insertions) relative to the complement of a sequence selected from SEQ ID NOs: 9013-9073.

In some embodiments, a targeting moiety comprises a protein, e.g., an RNA-binding protein, or functional fragment thereof. In some embodiments, such an RNA-binding protein binds to an ncRNA, e.g., eRNA or other RNA target described herein. Examples of RNA-binding proteins include, but are not limited to, Cas13 and Puf.

In some embodiments, a targeting moiety that targets a polypeptide component of a genomic or transcription complex may be or comprise a polypeptide agent (e.g., an antibody or antigen binding fragment thereof) that specifically binds with the target polypeptide component. In some embodiments, a targeting moiety targets, e.g., binds, a polypeptide component, e.g., a polypeptide that binds an eRNA, of a genomic or transcription complex. In some embodiments, a targeting moiety that targets a polypeptide is not or does not comprise a polypeptide. In some embodiments, a targeting moiety that targets a polypeptide comprises a polypeptide, e.g., an antibody or antigen binding fragment thereof. In some embodiments, a targeting moiety that targets a polypeptide, e.g., a polypeptide that binds an eRNA, comprises a small molecule or a nucleic acid (e.g., an oligonucleotide) that specifically binds with the targeted component. In some embodiments, a targeting moiety may comprise or further comprise (e.g., in addition to a nucleic acid) a non-antibody polypeptide, such as another protein (e.g., another genomic/transcription complex component, or a variant thereof) that interacts with the targeted component.

For example, in some embodiments, a targeting moiety comprises one or more of: a DNA binding small molecule (e.g., minor or major groove binders), peptide (e.g., zinc finger, TALEN, novel or modified peptide), protein (e.g., CTCF, modified CTCF with impaired CTCF binding and/or cohesion binding affinity), or nucleic acids (e.g., ssDNA, modified DNA or RNA, peptide oligonucleotide conjugates, locked nucleic acids, bridged nucleic acids, polyamides, peptide nucleic acids, and/or triplex forming oligonucleotides.

In some embodiments, a targeting moiety targets, e.g., binds, to a nucleic acid. Such a targeting moiety may comprise Synthetic Nucleic Acids (SNAs), Peptide Nucleic Acids (PNAs), Locked Nucleic Acids (LNAs), Bridged Nucleic Acids (BNAs), polyamide-SNA/LNA/BNA/PNA conjugates, DNA intercalating agents (e.g., SNA/LNA/BNA/PNA conjugates), and DNA sequence-specific binding peptide- or protein-SNA/LNA/PNA/BNA conjugates.

In some embodiments, a targeting moiety targets a ncRNA, e.g., eRNA, transcribed from ARID1A promoter. In some embodiments, a targeting moiety comprises a nucleic acid comprising a sequence at least 80, 85, 90, 91, 92, 93, 94, 95, 96, 97, 98, 99, or 100% identical to a sequence complementary to a ncRNA, e.g., eRNA, transcribed from ARID1A promoter. In some embodiments, a targeting moiety comprises a nucleic acid comprising a sequence with 8, 7, 6, 5, 4, 3, 2, 1, or no alterations relative to a sequence complementary to a ncRNA, e.g., eRNA, transcribed from ARID1A promoter. In some embodiments, a targeting moiety comprises a ribonucleic acid duplex that hybridizes with a ncRNA, e.g., eRNA, transcribed from ARID1A promoter, wherein the ncRNA:ribonucleic acid duplex complex is vulnerable to siRNA mediated degradation.

In some embodiments, a targeting moiety comprises a nucleic acid sequence that is at least 80, 85, 90, 91, 92, 93, 94, 95, 96, 97, 98, 99, or 100% identical to a sequence chosen from Tables 2-7. In some embodiments, a targeting moiety comprises a nucleic acid comprising a sequence with 8, 7, 6, 5, 4, 3, 2, 1, or no alterations relative to a sequence chosen from Tables 2-7.

### Effector Moiety

A modulating agent, e.g., fusion molecule, as described herein modulates (e.g., has an effect on) the structure and/or function of a targeted genomic complex or transcription complex, e.g., comprising an eRNA. In some embodiments the modulating agent comprises a targeting moiety, which, by binding a targeted component of the genomic/transcription complex (e.g., an eRNA or a component comprising an eRNA), achieves the modulation. In some embodiments, a modulating agent, e.g., fusion molecule, comprises a targeting moiety and an effector moiety, wherein the effector moiety contributes to or enhances the effect of the modulating agent. In some embodiments, the effector moiety adds to the effect that binding of the targeting moiety has, e.g., on the level or occupancy of a genomic complex or transcription complex or the expression of a target gene. In some embodiments, the effector moiety has functionality unrelated to the effect that binding of the targeting moiety has. For example, effector moieties may target, e.g., bind, a genomic sequence element (e.g., a genomic sequence element in or proximal to a genomic complex or transcription complex targeted by the targeting moiety).

In some embodiments, an effector moiety modulates a biological activity, e.g., increasing or decreasing an enzymatic activity, gene expression, cell signaling, and cellular or organ function. In some embodiments, an effector moiety binds a regulatory protein, e.g., which affects transcription or translation, thereby modulating the activity of the regulatory protein. In some embodiments, an effector moiety is an activator or inhibitor (or “negative effector”) as described herein. An effector moiety may also modulate protein stability/degradation and/or transcript stability/degradation. For example, an effector moiety may target a protein for ubiqutinylation or modulate (e.g., increase or decrease ubiquitinylation) the degradation of a target protein. In some embodiments, an effector moiety inhibits an enzymatic activity by blocking an enzyme's active site. For example, an effector moiety may be or comprise methotrexate, a structural analog of tetrahydrofolate, a coenzyme for dihydrofolate reductase that binds to dihydrofolate reductase 1000-fold more tightly than its natural substrate and inhibits nucleotide base synthesis.

In some embodiments, a modulating agent, e.g., fusion molecule, comprises a targeting moiety that binds a nucleic acid, e.g., eRNA, within a genomic complex or transcription complex (e.g., an anchor sequence-mediated conjunction), and is operably linked to an effector moiety that modulates the genomic complex or transcription complex.

In some embodiments, an effector moiety is a chemical, e.g., a chemical that modulates a cytosine (C) or an adenine (A) (e.g., Na bisulfite, ammonium bisulfite). In some embodiments, an effector moiety has enzymatic activity (e.g., methyl transferase, demethylase, nuclease (e.g., Cas9), or deaminase activity).

An effector moiety may be or comprise one or more of a small molecule, a peptide, a nucleic acid, a nanoparticle, an aptamer, or a pharmacoagent with poor PK/PD.

In some embodiments, a modulating agent, e.g., fusion molecule, comprises one effector moiety. In some embodiments, a modulating agent, e.g., fusion molecule, comprises more than one effector moiety. In some embodiments, a modulating agent, e.g., fusion molecule, comprises 1, 2, 3, 4, 5, 6, 7, 8, 9, 10, 11, 12, 13, 14, 15, 16, 17, 18, 19, 20, or more effector domains (and optionally, less than 20, 19, 18, 17, 16, 15, 14, 13, 12, 11, 10, 9, 8, 7, 6, 5, 4, 3, or 2 effector domains). For example, a modulating agent, e.g., fusion molecule, may comprise a plurality of enzymes with a role in DNA methylation (e.g., one or more methyltransferases, demethylases, or DNA topology modifying enzymes). In some embodiments, a modulating agent, e.g., fusion molecule, comprises a linker, e.g., an amino acid linker, connecting the targeting moiety and the effector moiety. In some embodiments, a linker comprises 2 or more amino acids, e.g., one or more GS sequences. In some embodiments wherein a modulating agent, e.g., fusion molecule, comprises a plurality of effector moieties, the modulating agent comprises linkers between each of the moieties.

In some embodiments, a modulating agent, e.g., fusion molecule, e.g., effector moiety, may comprise a peptide ligand, a full-length protein, a protein fragment, an antibody, an antibody fragment, and/or a targeting aptamer. In some embodiments, the protein of a modulating agent, e.g., fusion molecule, may bind a receptor such as an extracellular receptor, neuropeptide, hormone peptide, peptide drug, toxic peptide, viral or microbial peptide, synthetic peptide, or agonist or antagonist peptide.

In some embodiments, peptide or protein moieties a modulating agent, e.g., fusion molecule, e.g., effector moiety, may comprise antigens, antibodies, antibody fragments such as, e.g. single domain antibodies, ligands, and receptors such as, e.g., glucagon-like peptide-1 (GLP-1), GLP-2 receptor 2, cholecystokinin B (CCKB), and somatostatin receptor, peptide therapeutics such as, e.g., those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, and degradation or self-destruction peptides such as an apoptosis-inducing peptide signal or photosensitizer peptide.

Peptide or protein moieties as described herein may also include small antigen-binding peptides, e.g., antigen binding antibody or antibody-like fragments, such as, e.g., single chain antibodies, nanobodies (see, e.g., Steeland et al. 2016. Nanobodies as therapeutics: big opportunities for small antibodies. Drug Discov Today: 21(7):1076-113). Such small antigen binding peptides may bind, e.g. a cytosolic antigen, a nuclear antigen, an intra-organellar antigen.

In some aspects, a modulating agent, e.g., fusion molecule, e.g., effector moiety, comprises an antibody or fragment thereof (e.g., the targeting or effector moiety comprises an antibody). In some embodiments, gene expression is altered via use of effector moieties that are or comprise one or more antibodies or fragments thereof. In some embodiments, gene expression is altered via use of effector moieties that are or comprise one or more antibodies (or fragments thereof) and dCas9. In some embodiments, an antibody or fragment thereof is targeted to a particular genomic or transcription complex. In some embodiments, more than one antibody or fragment thereof (e.g., more than one of identical antibodies or one or more distinct antibodies (e.g., at least two antibodies, where each antibody is a different antibody)) is targeted to a particular genomic or transcription complex.

In some embodiments, gene expression is altered, e.g., decreased, via use of a modulating agent, e.g., fusion molecule, e.g., effector moiety, that comprises one or more antibodies or fragments thereof and dCas9. In some embodiments, one or more antibodies or fragments thereof is/are targeted to a particular genomic or transcription complex via dCas9 and target-specific guide RNA. In some embodiments, an antibody or fragment thereof for use in a modulating agent, e.g., fusion molecule. An antibody may be a fusion, a chimeric antibody, a non-humanized antibody, a partially or fully humanized antibody, etc. As will be understood by one of skill in the art, format of antibody(ies) used for targeting may be the same or different depending on a given target.

In some embodiments, a modulating agent, e.g., fusion molecule, e.g., effector moiety, comprises a conjunction nucleating molecule, a nucleic acid encoding a conjunction nucleating molecule, or a combination thereof. In some embodiments, an effector moiety comprises a conjunction nucleating molecule, a nucleic acid encoding a conjunction nucleating molecule, or a combination thereof.

A conjunction nucleating molecule may be, e.g., CTCF, cohesin, USF1, YY1, TATA-box binding protein associated factor 3 (TAF3), ZNF143 binding motif, or another polypeptide that promotes formation of an anchor sequence-mediated conjunction. A conjunction nucleating molecule may be an endogenous polypeptide or other protein, such as a transcription factor, e.g., autoimmune regulator (AIRE), another factor, e.g., X-inactivation specific transcript (XIST), or an engineered polypeptide that is engineered to recognize a specific DNA sequence of interest, e.g., having a zinc finger, leucine zipper or bHLH domain for sequence recognition. A conjunction nucleating molecule may modulate DNA interactions within or around the anchor sequence-mediated conjunction. For example, a conjunction nucleating molecule can recruit other factors to an anchor sequence that alters an anchor sequence-mediated conjunction formation or disruption.

A conjunction nucleating molecule may also have a dimerization domain for homo- or heterodimerization. One or more conjunction nucleating molecules, e.g., endogenous and engineered, may interact to form an anchor sequence-mediated conjunction. In some embodiments, a conjunction nucleating molecule is engineered to further include a stabilization domain, e.g., cohesion interaction domain, to stabilize an anchor sequence-mediated conjunction. In some embodiments, a conjunction nucleating molecule is engineered to bind a target sequence, e.g., target sequence binding affinity is modulated. In some embodiments, a conjunction nucleating molecule is selected or engineered with a selected binding affinity for an anchor sequence within an anchor sequence-mediated conjunction. Conjunction nucleating molecules and their corresponding anchor sequences may be identified through use of cells that harbor inactivating mutations in CTCF and Chromosome Conformation Capture or 3C-based methods, e.g., Hi-C or high-throughput sequencing, to examine topologically associated domains, e.g., topological interactions between distal DNA regions or loci, in the absence of CTCF. Long-range DNA interactions may also be identified. Additional analyses may include ChIA-PET analysis using a bait, such as Cohesin, YY1 or USF1, ZNF143 binding motif, and MS to identify complexes that are associated with a bait.

In some embodiments, a modulating agent, e.g., fusion molecule, e.g., effector moiety, comprises a DNA-binding domain of a protein. In some such embodiments, the targeting moiety of the modulating agent may be or comprise the DNA-binding domain. In some embodiments, one or more of a targeting moiety and/or an effector moiety is or comprises a DNA-binding domain.

In some embodiments, DNA binding domains enhance or alter effect of targeting of a modulating agent, e.g., fusion molecule, but do not alone achieve complete targeting by a modulating agent. In some embodiments, DNA binding domains enhance targeting of a modulating agent, e.g., fusion molecule. In some embodiments, DNA binding domains enhance efficacy of a modulating agent, e.g., fusion molecule. DNA-binding proteins have distinct structural motifs that play a key role in binding DNA. A helix-turn-helix (HTH) motif is a common DNA recognition motif in repressor proteins. Such a motif comprises two helices, one of which recognizes DNA (aka recognition helix) with side chains providing binding specificity. Such motifs are commonly used to regulate proteins that are involved in developmental processes. Sometimes more than one protein competes for the same sequence or recognizes the same DNA fragment. Different proteins may differ in their affinity for the same sequence, or DNA conformation, respectively through H-bonds, salt bridges and Van der Waals interactions. DNA-binding proteins with a helix-hairpin-helix HhH structural motif may be involved in non-sequence-specific DNA binding that occurs via the formation of hydrogen bonds between protein backbone nitrogens and DNA phosphate groups.

DNA-binding proteins with an HLH structural motif are transcriptional regulatory proteins and are principally related to a wide array of developmental processes. An HLH structural motif is longer, in terms of residues, than HTH or HhH motifs. Many of these proteins interact to form homo- and hetero-dimers. A structural motif is composed of two long helix regions, with an N-terminal helix binding to DNA, while a complex region allows the protein to dimerize.

In some transcription factors, a dimer binding site with DNA forms a leucine zipper. This motif includes two amphipathic helices, one from each subunit, interacting with each other resulting in a left handed coiled-coil super secondary structure. A leucine zipper is an interdigitation of regularly spaced leucine residues in one helix with leucines from an adjacent helix. Mostly, helices involved in leucine zippers exhibit a heptad sequence (abcdefg) with residues a and d being hydrophobic and other residues being hydrophilic. Leucine zipper motifs can mediate either homo- or heterodimer formation. Some eukaryotic transcription factors show a unique motif called a Zn-finger, where a Zn⁺⁺ ion is coordinated by 2 Cys and 2 His residues. Such a transcription factor includes a trimer with the stoichiometry ββ′α. An apparent effect of Zn⁺⁺ coordination is stabilization of a small complex structure instead of hydrophobic core residues. Each Zn-finger interacts in a conformationally identical manner with successive triple base pair segments in the major groove of the double helix. Protein-DNA interaction is determined by two factors: (i) H-bonding interaction between α-helix and DNA segment, mostly between Arg residues and Guanine bases. (ii) H-bonding interaction with DNA phosphate backbone, mostly with Arg and His. An alternative Zn-finger motif chelates Zn⁺⁺ with 6 Cys. DNA-binding proteins also include TATA box binding proteins (TBP), first identified as a component of the class II initiation factor TFIID. These binding proteins participate in transcription by all three nuclear RNA polymerases acting as subunit in each of them. Structure of TBP shows two α/β structural domains of 89-90 amino acids. The C-terminal or core region of TBP binds with high affinity to a TATA consensus sequence (TATAa/tAa/t, SEQ ID NO: 3) recognizing minor groove determinants and promoting DNA bending. TBP resemble a molecular saddle. The binding side is lined with central 8 strands of a 10-stranded anti-parallel β-sheet. The upper surface contains four α-helices and binds to various components of transcription machinery.

DNA provides base specificity via nitrogen bases. R-groups of amino acids, with basic residues such as Lysine, Arginine, Histidine, Asparagine and Glutamine can easily interact with adenine of an A: T base pair, and guanine of a G: C base pair, where NH2 and X═O groups of base pairs can preferably form hydrogen bonds with amino acid residues of Glutamine, Aspargine, Arginine and Lysine.

In some embodiments, a DNA-binding protein is a transcription factor. Transcription factors (TFs) may be modular proteins containing a DNA-binding domain that is responsible for specific recognition of base sequences and one or more effector domains that can activate or repress transcription. TFs interact with chromatin and recruit protein complexes that serve as coactivators or corepressors. In some embodiments, a modulating agent, e.g., a fusion molecule, e.g., the effector moiety of a fusion molecule, comprises one or more RNAs (e.g. gRNA) and dCas9. In some embodiments, one or more RNAs is/are targeted to particular genomic or transcription complexes via dCas9 and target-specific guide RNA. As will be understood by one of skill in the art, RNAs used for targeting may be the same or different depending on a given target.

In some embodiments, gene expression is altered via use of a modulating agent, e.g., fusion molecule, comprising an effector moiety, that comprises an antibody or fragment thereof and dCas9. In some embodiments, one or more RNAs is/are targeted to a particular genomic complex via dCas9 and target-specific guide RNA.

In some embodiments, a modulating agent, e.g., fusion molecule, e.g., the effector moiety of a fusion molecule, comprises a nucleic acid sequence, e.g., a guide RNA (gRNA). In some embodiments, a modulating agent, e.g., fusion molecule, e.g., the effector moiety of a fusion molecule, comprises a guide RNA or nucleic acid encoding the guide RNA. A gRNA is a short synthetic RNA composed of a “scaffold” sequence necessary for Cas9-binding and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

In some embodiments, a gRNA is complementary to a nucleic acid participating in a genomic or transcription complex, e.g., a genomic sequence element (e.g., anchor sequence) or a ncRNA (e.g., eRNA).

In some embodiments, a gRNA is complementary to part of a genomic complex or transcription complex. In some embodiments, a gRNA is complementary to a genomic sequence element. In some embodiments, a gRNA is complementary to genomic sequence that is not itself part of a genomic complex or transcription complex (e.g., an anchor sequence-mediated conjunction). For example, in some such embodiments, a gRNA may be complementary to genomic sequence encoding a transcription factor, wherein the transcription factor is part of a target genomic complex, but the genomic sequence encoding the transcription factor is, e.g. on a different chromosome.

In some embodiments, an epigenetic modifying moiety comprises a gRNA, antisense DNA, or triplex forming oligonucleotide used as a DNA target and steric presence in the vicinity of the genomic complex or transcription complex, e.g., in the vicinity of the anchoring sequence. A gRNA recognizes specific DNA sequences (e.g., an anchor sequence, a CTCF anchor sequence, flanked by sequences that confer sequence specificity). A gRNA may include additional sequences that interfere with conjunction nucleating molecule sequence to act as a steric blocker. In some embodiments, a gRNA is combined with one or more peptides, e.g., S-adenosyl methionine (SAM), that acts as a steric presence to interfere with a conjunction nucleating molecule.

In some embodiments, a modulating agent, e.g., fusion molecule, e.g., effector moiety, comprises an RNAi molecule. Certain RNA agents can inhibit gene expression through a biological process using RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207). In some embodiments, the present disclosure provides compositions to inhibit expression of a gene encoding a polypeptide described herein, e.g., a conjunction nucleating molecule or epigenetic modifying agent.

RNAi molecules comprise a sequence substantially complementary, or fully complementary, to all or a fragment of a target gene. RNAi molecules may complement sequences at a boundary between introns and exons to prevent maturation of newly-generated nuclear RNA transcripts of specific genes into mRNA for transcription. RNAi molecules complementary to specific genes can hybridize with an mRNA for that gene and prevent its translation. An antisense molecule can be, for example, DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG). An antisense molecule may be comprised of synthetic nucleotides.

RNAi molecules can be provided to the cell as “ready-to-use” RNA synthesized in vitro or as an antisense gene transfected into cells which will yield RNAi molecules upon transcription. Hybridization with mRNA results in degradation of a hybridized molecule by RNAse H and/or inhibition of formation of translation complexes. Both result in a failure to produce a product of an original gene.

Length of an RNAi molecule that hybridizes to a transcript of interest should be around 10 nucleotides, between about 15 or 30 nucleotides, or about 15, 16, 17, 18, 19, 20, 21, 22, 23, 24, 25, 26, 27, 28, 29, 30 or more nucleotides. Degree of identity of an antisense sequence to a targeted transcript should be at least 75%, at least 80%, at least 85%, at least 90%, or at least 95%.

RNAi molecules may also comprise overhangs, i.e. typically unpaired, overhanging nucleotides which are not directly involved in a double helical structure normally formed by a core sequences of herein defined pair of sense strand and antisense strand. RNAi molecules may contain 3′ and/or 5′ overhangs of about 1-5 bases independently on each of a sense and antisense strand. In some embodiments, both sense and antisense strands contain 3′ and 5′ overhangs. In some embodiments, one or more 3′ overhang nucleotides of one strand base (e.g. sense) pairs with one or more 5′ overhang nucleotides of the other strand (e.g. antisense). In some embodiments, one or more 3′ overhang nucleotides of one strand base (e.g. sense) do not pair with the one or more 5′ overhang nucleotides of the other strand (e.g. antisense). Sense and antisense strands of an RNAi molecule may or may not contain the same number of nucleotide bases. Antisense and sense strands may form a duplex wherein a 5′ end only has a blunt end, a 3′ end only has a blunt end, both a 5′ and 3′ ends are blunt ended, or neither a 5′ end nor the 3′ end are blunt ended. In some embodiments, one or more nucleotides in an overhang contains a thiophosphate, phosphorothioate, deoxynucleotide inverted (3′ to 3′ linked) nucleotide or is a modified ribonucleotide or deoxynucleotide.

Small interfering RNA (siRNA) molecules comprise a nucleotide sequence that is identical to about 15 to about 25 contiguous nucleotides of a target mRNA. In some embodiments, an siRNA sequence commences with a dinucleotide AA, comprises a GC-content of about 30-70% (about 30-60%, about 40-60%, or about 45%-55%), and does not have a high percentage identity to any nucleotide sequence other than a target in a genome of a mammal in which it is to be introduced, for example as determined by standard BLAST search.

siRNAs and shRNAs resemble intermediates in processing pathway(s) of endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave an mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from an miRNA's 5′ end (Rajewsky, Nat Genet 38 Suppl:S8-13, 2006; Lim et al., Nature 433:769-773, 2005). This region is known as a seed region. Because siRNAs and miRNAs are interchangeable, exogenous siRNAs downregulate mRNAs with seed complementarity to an siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in relevant literature. RNAi molecules are readily designed and produced by technologies known in the art. In addition, there are computational tools that increase chances of finding effective and specific sequence motifs (Pei et al. 2006, Reynolds et al. 2004, Khvorova et al. 2003, Schwarz et al. 2003, Ui-Tei et al. 2004, Heale et al. 2005, Chalk et al. 2004, Amarzguioui et al. 2004).

The RNAi molecule modulates expression of RNA encoded by a gene. Because multiple genes can share some degree of sequence homology with each other, in some embodiments, the RNAi molecule can be designed to target a class of genes with sufficient sequence homology. In some embodiments, an RNAi molecule can contain a sequence that has complementarity to sequences that are shared amongst different gene targets or are unique for a specific gene target. In some embodiments, an RNAi molecule can be designed to target conserved regions of an RNA sequence having homology between several genes thereby targeting several genes in a gene family (e.g., different gene isoforms, splice variants, mutant genes, etc.). In some embodiments, an RNAi molecule can be designed to target a sequence that is unique to a specific RNA sequence of a single gene.

In some embodiments, an RNAi molecule targets a sequence encoding a component of a genomic complex or transcription complex, e.g., a conjunction nucleating molecule, e.g., CTCF, cohesin, USF1, YY1, TATA-box binding protein associated factor 3 (TAF3), ZNF143, or another polypeptide that promotes the formation of an anchor sequence-mediated conjunction, or an epigenetic modifying agent, e.g., an enzyme involved in post-translational modifications including, but are not limited to, DNA methylases (e.g., DNMT3a, DNMT3b, DNMTL), DNA demethylation (e.g., the TET family enzymes catalyze oxidation of 5-methylcytosine to 5-hydroxymethylcytosine and higher oxidative derivatives), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), protein-lysine N-methyltransferase (SMYD2), and others. In some embodiments, the RNAi molecule targets a protein deacetylase, e.g., sirtuin 1, 2, 3, 4, 5, 6, or 7. In some embodiments, the present disclosure provides a composition comprising an RNAi that targets a conjunction nucleating molecule, e.g., CTCF.

In some embodiments, an RNAi molecule targets a nucleic acid sequence that is part of a genomic complex or transcription complex (e.g. ncRNA, e.g., eRNA). In some embodiments, a modulating agent, e.g., fusion molecule, e.g., the targeting moiety or effector moiety of a fusion molecule, comprises an RNAi molecule that targets an eRNA that is part of a genomic complex or transcription complex. A modulating agent, e.g., fusion molecule, e.g., effector moiety, may comprise an aptamer, such as an oligonucleotide aptamer or a peptide aptamer. Aptamer moieties are oligonucleotide or peptide aptamers.

A modulating agent, e.g., fusion molecule, e.g., effector moiety, may comprise an oligonucleotide aptamer. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers possess desirable storage properties, and elicit little or no immunogenicity in therapeutic applications.

Both DNA and RNA aptamers show robust binding affinities for various targets. For example, DNA and RNA aptamers have been selected for t lysozyme, thrombin, human immunodeficiency virus trans-acting responsive element (HIV TAR), https://en.wikipedia.org/wiki/Aptamer-cite_note-10 hemin, interferon γ, vascular endothelial growth factor (VEGF), prostate specific antigen (PSA), dopamine, and the non-classical oncogene, heat shock factor 1 (HSF1).

Diagnostic techniques for aptamer based plasma protein profiling includes aptamer plasma proteomics. This technology will enable future multi-biomarker protein measurements that can aid diagnostic distinction of disease versus healthy states.

A modulating agent, e.g., fusion molecule, e.g., effector moiety, may comprise a peptide aptamer moiety. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide complexes of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer complex attached to a transcription factor binding domain is screened against a target protein attached to a transcription factor activating domain. In vivo binding of a peptide aptamer to its target via this selection strategy is detected as expression of a downstream yeast marker gene. Such experiments identify particular proteins bound by aptamers, and protein interactions that aptamers disrupt, to cause a given phenotype. In addition, peptide aptamers derivatized with appropriate functional moieties can cause specific post-translational modification of their target proteins, or change subcellular localization of the targets.

Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently a yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. Peptides panned from combinatorial peptide libraries have been stored in a special database with named MimoDB.

Effector Moieties that Negatively Effect Genomic/Transcription Complexes

In some embodiments, an effector moiety reduces the level of a genomic complex or transcription complex, e.g., an anchor sequence-mediated conjunction, (e.g., when a cell has been contacted with a modulating agent (e.g., fusion molecule) comprising the effector moiety, or when the effector moiety has been co-localized to the genomic complex component or transcription factor by the targeting moiety) as compared with when it is absent. In some embodiments, the level of a genomic complex or transcription complex decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., fusion molecule, comprising the effector moiety relative to the absence of said modulating agent. In some embodiments, the presence of the effector moiety alters, e.g., decreases, occupancy of the genomic complex or transcription complex at a genomic sequence element (e.g., a target gene, or an enhancer associated with a targeted eRNA). In some embodiments, occupancy decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., fusion molecule, comprising the effector moiety relative to the absence of said modulating agent.

In some embodiments, the occupancy of a genomic complex or transcription complex at a genomic sequence element (e.g., a gene, promoter, or enhancer, e.g., associated with the geonmic or transcription complex) is decreased in the presence of a modulating agent, e.g., fusion molecule, comprising the effector moiety relative to the absence of said modulating agent. In some embodiments, the presence of the effector moiety alters, e.g., decreases, occupancy of the genomic complex or transcription complex at a genomic sequence element by at least 10, 20, 30, 40, 50, 60, 70, 80, 90, or 100% (and optionally, up to 100, 90, 80, 70, 60, 50, 40, 30, or 20%) in the presence of a modulating agent, e.g., fusion molecule, comprising the effector moiety relative to the absence of said modulating agent.

In some embodiments, the occupancy of a targeted component in/at the genomic complex or transcription complex is decreased in the presence of a modulating agent, e.g., fusion molecule, comprising the effector moiety relative to the absence of said modulating agent. In some embodiments, the presence of the effector moiety alters, e.g., decreases, occupancy of a targeted component in/at the genomic complex or transcription complex by at least 10, 20, 30, 40, 50, 60, 70, 80, 90, or 100% (and optionally, up to 100, 90, 80, 70, 60, 50, 40, 30, or 20%) in the presence of a modulating agent, e.g., fusion molecule, comprising the effector moiety relative to the absence of said modulating agent.

In some embodiments, a modulating agent, e.g., fusion molecule, that disrupts an interaction between a genomic sequence element and another genomic complex component or transcription factor comprises a effector moiety that decreases the dimerization of an endogenous nucleating polypeptide when present as compared with when the effector moiety is absent. In some embodiments, the change in dimerization and/or disruption of interaction are determined using ChIP-Seq.

In some embodiments, a effector moiety alters, e.g., decreases, the level of a genomic complex or transcription complex comprising a targeted component. In some embodiments, the change in genomic complex level and/or disruption of interaction are determined using ChIP-Seq.

In some embodiments, a effector moiety alters, e.g., decreases, the expression of a target gene associated with the genomic complex or transcription complex comprising a targeted component. In some embodiments, the expression of the target gene decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., fusion molecule, comprising the effector moiety relative to the absence of said modulating agent.

In some embodiments, a modulating agent, e.g., fusion molecule, comprises a targeting moiety that targets, e.g., binds, a nucleic acid component of a genomic complex or transcription complex (e.g., eRNA), and an effector moiety that provides a steric presence (e.g., to inhibit binding of another genomic complex component or transcription factor, e.g., to a component that binds the eRNA). An effector moiety may comprise a dominant negative binding molecule or fragment thereof (e.g., a protein that recognizes and binds a genomic complex component (e.g., a genomic sequence element, e.g., an anchor sequence, (e.g., a CTCF binding motif)) or transcription factor, but with an alteration (e.g., mutation) preventing formation of a functional transcription factor or genomic complex), a polypeptide that interferes with transcription factor binding or function (e.g., contact between a transcription factor and its target sequence to be transcribed), a nucleic acid sequence ligated to a small molecule that imparts steric interference, or any other combination of a recognition element and a steric blocker.

An exemplary effector moiety may include, but is not limited to: ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3a, DNMT3b, DNMTL), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdb1), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDM1A and lysine-specific histone demethylase 1 (LSD1), helicases such as DHX9, acetyltransferases, deacetylases (e.g., sirtuin 1, 2, 3, 4, 5, 6, or 7), kinases, phosphatases, DNA-intercalating agents such as ethidium bromide, SYBR green, and proflavine, efflux pump inhibitors such as peptidomimetics like phenylalanine arginyl β-naphthylamide or quinoline derivatives, nuclear receptor activators and inhibitors, proteasome inhibitors, competitive inhibitors for enzymes such as those involved in lysosomal storage diseases, protein synthesis inhibitors, nucleases (e.g., Cpf1, Cas9, zinc finger nuclease), fusions of one or more thereof (e.g., dCas9-DNMT, dCas9-APOBEC, dCas9-UG1), and specific domains from proteins, such as KRAB domain.

Genetic Modifying Moieties

In some embodiments, a modulating agent (e.g., fusion molecule) comprises an effector moiety that is or comprises a genetic modifying moiety (e.g., components of a gene editing system). In some embodiments, a genetic modifying moiety comprises one or more components of a gene editing system. Genetic modifying moieties may be used in a variety of contexts including but not limited to gene editing. For example, such moieties may be used to localize an effector moiety to a genetic locus, e.g., so that the modulating agent, e.g., effector moiety, may physically modify, genetically modify, and/or epigenetically modify a target sequences, e.g., anchor sequence.

In some embodiments, a genetic modifying moiety may target one or more nucleotides, such as through a gene editing system, of a sequence, e.g., an ncRNA such as an eRNA. In some embodiments, a genetic modifying moiety binds an ncRNA such as an eRNA and alters a genomic or transcription complex, e.g., alters topology of an anchor sequence-mediated conjunction.

In some embodiments, a genetic modifying moiety targets one or more nucleotides of genomic DNA, e.g., such as through CRISPR, TALEN, dCas9, oligonucleotide pairing, recombination, transposon, within or as a component of a genomic or transcription complex (e.g. within an anchor sequence-mediated conjunction) for substitution, addition or deletion.

In some embodiments, a genetic modifying moiety introduces a targeted alteration into one or more nucleotides of genomic DNA within a genomic or transcription complex, wherein the alteration modulates transcription of a gene, e.g., in a human cell. In some embodiments, a genetic modifying moiety introduces a targeted alteration into an ncRNA or eRNA that is part of a genomic or transcription complex (e.g., an anchor sequence-mediated conjunction), wherein the alteration modulates transcription of a gene associated with the genomic or transcription complex. A targeted alteration may include a substitution, addition, or deletion of one or more nucleotides.

Exemplary gene editing systems whose components may be suitable for use in genetic modifying moieties include clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 Jul. 30, 46:1-8; and Zheng et al., Precise gene deletion and replacement using the CRISPR/Cas9 system in human cells. BioTechniques, Vol. 57, No. 3, September 2014, pp. 115-124.

For example, in some embodiments, a genetic modifying moiety is site-specific and comprises a Cas nuclease (e.g., Cas9) and a site-specific guide RNA, as described further herein. In some embodiments, a genetic modifying moiety comprises a Cas nuclease (e.g., Cas9), a site-specific guide RNA and an effector domain (e.g., epigenome editors including but not restricted to: DNMT3a, DNMT3L, DNMT3b, KRAB domain, Tet1, p300, VP64. In some embodiments, a Cas nuclease is enzymatically inactive, e.g., a dCas9, as described further herein.

In some embodiments, methods and compositions as provided herein can be used with a CRISPR-based gene editing, whereby guide RNA (gRNA) are used in a clustered regulatory interspaced short palindromic repeat (CRISPR) system for gene editing. CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. For example, in a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. crRNA also contains a region that binds to the tracrRNA to form a partially double-stranded structure which is cleaved by RNase III, resulting in a crRNA/tracrRNA hybrid. A crRNA/tracrRNA hybrid then directs Cas9 endonuclease to recognize and cleave a target DNA sequence. A target DNA sequence must generally be adjacent to a “protospacer adjacent motif” (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPRI), 5′-NGGNG (Streptococcus thermophilus CRISPR3), and 5′-NNNGATT (Neisseria meningitidis). Some endonucleases, e.g., Cas9 endonucleases, are associated with G-rich PAM sites, e. g., 5′-NGG, and perform blunt-end cleaving of the target DNA at a location 3 nucleotides upstream from (5′ from) the PAM site. Another class TT CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words, a Cpf1 system requires only Cpf1 nuclease and a crRNA to cleave a target DNA sequence. Cpf1 endonucleases, are associated with T-rich PAM sites, e. g., 5′-TTN. Cpf1 can also recognize a 5′-CTA PAM motif. Cpf1 cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a modulating agent includes a sequence targeting polypeptide, such as an enzyme, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the Cas protein is modified to deactivate the nuclease, e.g., nuclease-deficient Cas9, and to recruit transcription activators or repressors, e.g., the ω-subunit of the E. coli Pol, VP64, the activation domain of p65, KRAB, or SID4X, to induce epigenetic modifications, e.g., histone acetyltransferase, histone methyltransferase and demethylase, DNA methyltransferase and enzyme with a role in DNA demethylation (e.g., the TET family enzymes catalyze oxidation of 5-methylcytosine to 5-hydroxymethylcytosine and higher oxidative derivatives).

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence are required by Cas9 for DNA cleavage to occur; for Cpf1 at least about 16 nucleotides of gRNA sequence is needed to achieve detectable DNA cleavage.

Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut target DNA but interferes with transcription by steric hindrance. dCas9 can further be fused with a heterologous effector to repress (CRISPRi) or activate (CRISPRa) expression of a target gene. For example, Cas9 can be fused to a transcriptional silencer (e.g., a KRAB domain) or a transcriptional activator (e.g., a dCas9-VP64 fusion). A catalytically inactive Cas9 (dCas9) fused to FokI nuclease (“dCas9-FokI”) can be used to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, Mass. 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, a genetic modifying moiety may comprise a polypeptide (e.g. peptide or protein moiety) linked to a gRNA and a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. Choice of nuclease and gRNA(s) is determined by whether a targeted mutation is a deletion, substitution, or addition of nucleotides, e.g., a deletion, substitution, or addition of nucleotides to a targeted sequence. Fusions of a catalytically inactive endonuclease, e.g., a dead Cas9 (dCas9, e.g., D10A; H840A) tethered with all or a portion of (e.g., biologically active portion of) an (one or more) effector domain (e.g., epigenome editors including but not restricted to: DNMT3a, DNMT3L, DNMT3b, KRAB domain, Tet1, p300, VP64 and fusions of the aforementioned) create himeric proteins that can be linked to a polypeptide to guide a provided composition to specific DNA sites by one or more RNA sequences (e.g., DNA recognition elements including, but not restricted to zinc finger arrays, sgRNA, TAL arrays, peptide nucleic acids described herein) to modulate activity and/or expression of one or more target nucleic acids sequences (e.g., to methylate or demethylate a DNA sequence).

As used herein, a “biologically active portion of an effector domain” is a portion that maintains function (e.g. completely, partially, minimally) of an effector domain (e.g., a “minimal” or “core” domain). In some embodiments, fusion of a dCas9 with all or a portion of one or more effector domains of an epigenetic modifying agent (such as a DNA methylase or enzyme with a role in DNA demethylation, e.g., DNMT3a, DNMT3b, DNMT3L, a DNMT inhibitor, combinations thereof, TET family enzymes, protein acetyl transferase or deacetylase, dCas9-DNMT3a/3L, dCas9-DNMT3a/3L/KRAB, dCas9/VP64) creates a chimeric protein that is linked to the polypeptide and useful in the methods described herein. An effector moiety comprising such a chimeric protein is referred to as either a genetic modifying moiety (because of its use of a gene editing system component, Cas9) or an epigenetic modifying moiety (because of its use of an effector domain of an epigenetic modifying agent).

In some embodiments, a genetic modifying moiety comprises one or more components of a CRISPR system described herein.

In some embodiments, provided technologies are described as comprising a gRNA that specifically targets a target gene. In some embodiments, the target gene is an oncogene, a tumor suppressor, or a a nucleotide repeat disease related gene.

In some embodiments, technologies provided herein include methods of delivering one or more genetic modifying moieties (e.g., CRISPR system components) described herein to a subject, e.g., to a nucleus of a cell or tissue of a subject, by linking such a moiety to a targeting moiety as part of a fusion molecule.

Epigenetic Modifying Moieties

In some embodiments, an effector moiety is or comprises an epigenetic modifying moiety that modulates the two-dimensional structure of chromatin (i.e., that modulate structure of chromatin in a way that would alter its two-dimensional representation).

Epigenetic modifying moieties useful in methods and compositions of the present disclosure include agents that affect, e.g., DNA methylation, histone acetylation, and RNA-associated silencing. In some embodiments, methods provided herein involve sequence-specific targeting of an epigenetic enzyme (e.g., an enzyme that generates or removes epigenetic marks, e.g., acetylation and/or methylation). Exemplary epigenetic enzymes that can be targeted to a genomic sequence element as described herein include DNA methylases (e.g., DNMT3a, DNMT3b, DNMTL), DNA demethylation (e.g., the TET family), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012):1-18.

In some embodiments, an epigenetic modifying moiety comprises a histone methyltransferase activity (e.g., a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof). In some embodiments, an epigenetic modifying moiety comprises a histone demethylase activity (e.g., a protein chosen from KDM1A (i.e., LSD1), KDM1B (i.e., LSD2), KDM2A, KDM2B, KDM5A, KDM5B, KDM5C, KDM5D, KDM4B, N066, or a functional variant or fragment of any thereof). In some embodiments, an epigenetic modifying moiety comprises a histone deacetylase activity (e.g., a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof). In some embodiments, an epigenetic modifying moiety comprises a DNA methyltransferase activity (e.g., a protein chosen from MQl, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof). In some embodiments, an epigenetic modifying moiety comprises a DNA demethylase activity (e.g., a protein chosen from TET1, TET2, TET3, or TDG, or a functional variant or fragment of any thereof). In some embodiments, an epigenetic modifying moiety comprises a transcription repressor activity (e.g., a protein chosen from KRAB, MeCP2, HP1, RBBP4, REST, FOGI, SUZ12, or a functional variant or fragment of any thereof).

In some embodiments, an epigenetic modifying moiety useful herein comprises a construct described in Koferle et al. Genome Medicine 7.59 (2015):1-3 (e.g., at Table 1), incorporated herein by reference. For example, in some embodiments, an expression repressor comprises or is a construct found in Table 1 of Koferle et al., e.g., a histone acetyltransferase, histone deacetylase, histone methyltransferase, DNA demethylation, or H3K4 and/or H3K9 histone demethylase described in Table 1 (e.g., dCas9-p300, TALE-TET1, ZF-DNMT3A, or TALE-LSD1).

### Fusion Molecules

In some embodiments, a modulating agent of the present disclosure may be or comprise a fusion molecule, such as a fusion molecule that comprises two or more moieties. In some embodiments, a fusion molecule comprises one or more moieties described herein, e.g., a targeting moiety and/or effector moiety.

For example, in some embodiments, provided compositions are fusion molecules comprising a targeting moiety (such as any one of the targeting moieties as described herein) and an effector moiety comprising a deaminating agent, wherein a targeting moiety targets a fusion molecule to a target genomic or transcription complex, e.g., by binding specifically to an eRNA associated with said target genomic or transcription complex. A variety of deaminating agents can be used, such as deaminating agents that do not have enzymatic activity (e.g., chemical agents such as sodium bisulfite), and/or deaminating agents that have enzymatic activity (e.g., a deaminase or functional portion thereof).

In some aspects, the present disclosure provides modulating agents, e.g., a fusion molecule, comprising a domain, e.g., an enzyme domain, that acts on DNA (e.g., a nuclease domain, e.g., a Cas9 domain, e.g., a dCas9 domain; a DNA methyltransferase, a demethylase, a deaminase), in combination with at least one guide RNA (gRNA) or antisense DNA oligonucleotide that targets the fusion molecule to an anchor sequence of a target anchor sequence-mediated conjunction or an eRNA associated with a genomic or transcription complex (e.g., that comprises an anchor sequence). In some embodiments, an enzyme domain is a Cas9 or a dCas9. In some embodiments, a fusion molecule comprises two enzyme domains, e.g., a dCas9 and a methylase or demethylase domain.

In some aspects, the present disclosure provides modulating agents, e.g., a fusion molecule, comprising a domain, e.g., an enzyme domain, that acts on DNA (e.g., a nuclease domain, e.g., a Cas9 domain, e.g., a dCas9 domain; a DNA methyltransferase, a demethylase, a deaminase), in combination with at least one guide RNA (gRNA) or antisense DNA oligonucleotide that targets the fusion molecule to sequence within a genomic complex that is not an anchor sequence. In some embodiments, targeting by the fusion molecule (e.g., binding and/or the localized biological activity of the fusion molecule) is effective to alter, in a human cell, a genomic or transcription complex, e.g., anchor sequence-mediated conjunction. In some embodiments, a sequence is targeted to a component of a genomic or transcription complex that is or comprises an ncRNA, e.g., eRNA. In some embodiments, an enzyme domain is a Cas9 or a dCas9. In some embodiments, a fusion molecule comprises two enzyme domains, e.g., a dCas9 and a methylase or demethylase domain.

In some aspects, the present disclosure provides methods of modulating expression of a gene by administering a composition comprising a fusion molecule, e.g., protein fusion, described herein. In some embodiments, for example, a fusion molecule may comprise (e.g., as part of an effector or targeting moiety) dCas9-DNMT (e.g., comprises dCas9 and DNMT as part of the same polypeptide chain), dCas9-DNMT-3a-3L, dCas9-DNMT-3a-3a, dCas9-DNMT-3a-3L-3a, dCas9-DNMT-3a-3L-KRAB, dCas9-KRAB, dCas9-APOBEC, APOBEC-dCas9, dCas9-APOBEC-UGI, dCas9-UGI, UGI-dCas9-APOBEC, UGI-APOBEC-dCas9, any variation of protein fusions as described herein, or other fusions of proteins or protein domains described herein.

Exemplary dCas9 fusion methods and compositions that are adaptable to methods and compositions provided by the present disclosure are known and are described, e.g., in Kearns et al., Functional annotation of native enhancers with a Cas9-histone demethylase fusion. Nature Methods 12, 401-403 (2015); and McDonald et al., Reprogrammable CRISPR/Cas9-based system for inducing site-specific DNA methylation. Biology Open 2016: doi: 10.1242/bio.019067. Using methods known in the art, dCas9 can be fused to any of a variety of agents and/or molecules as described herein; such resulting fusion molecules can be useful in various disclosed methods.

In some embodiments, a modulating agent, e.g., fusion molecule, may be or comprise a peptide oligonucleotide conjugate moiety or entity. Peptide oligonucleotide conjugates include chimeric molecules comprising a nucleic acid moiety linked to a peptide moiety (such as a peptide/nucleic acid mixmer). In some embodiments, a peptide moiety may include any peptide or protein moiety described herein. In some embodiments, a nucleic acid moiety may include any nucleic acid or oligonucleotide, e.g., DNA or RNA or modified DNA or RNA, described herein.

In some embodiments, a peptide oligonucleotide conjugate comprises a peptide antisense oligonucleotide conjugate. In some embodiments, a peptide oligonucleotide conjugate is a synthetic oligonucleotide with a chemically modified backbone. A peptide oligonucleotide conjugate can bind to both DNA and RNA targets in a sequence-specific manner to form a duplex structure. When bound to double-stranded DNA (dsDNA) target, a peptide oligonucleotide conjugate replaces one DNA strand in a duplex by strand invasion to form a triplex structure and a displaced DNA strand may exist as a single-stranded D-loop.

In some embodiments, a peptide oligonucleotide conjugate may be cell- and/or tissue-specific. In some embodiments, such a conjugate may be conjugated directly to, e.g. oligos, peptides, and/or proteins, etc.

In some embodiments, a peptide oligonucleotide conjugate comprises a membrane translocating polypeptide, for example, membrane translocating polypeptides as described elsewhere herein.

Solid-phase synthesis of several peptide-oligonucleotide conjugates has been described in, for example, Williams, et al., 2010, Curr. Protoc. Nucleic Acid Chem., Chapter Unit 4.41, doi: 10.1002/0471142700.nc0441s42. Synthesis and characterization of very short peptide-oligonucleotide conjugates and stepwise solid-phase synthesis of peptide-oligonucleotide conjugates on new solid supports have been described in, for example, Bongardt, et al., Innovation Perspect. Solid Phase Synth. Comb. Libr., Collect. Pap., Int. Symp., 5th, 1999, 267-270; Antopolsky, et al., Helv. Chim. Acta, 1999, 82, 2130-2140.

In some embodiments, provided compositions are pharmaceutical compositions comprising fusion molecules as described herein.

In some aspects, the present disclosure provides cells or tissues comprising fusion molecules as described herein.

In some aspects, the present disclosure provides pharmaceutical compositions comprising fusion molecules as described herein.

### Linkers

In some embodiments, modulating agents, e.g., fusion molecules, may include one or more linkers. In some embodiments, a modulating agent, e.g., fusion molecule, comprising a first moiety and a second moiety has a linker between the first and second moieties, e.g., between a targeting moiety and an effector moiety. A linker may be a chemical bond, e.g., one or more covalent bonds or non-covalent bonds. In some embodiments linkers are covalent. In some embodiments, linkers are non-covalent. In some embodiments, a linker is a peptide linker. Such a linker may be between 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 amino acids in length, or greater than or equal to 2, 5, 10, 15, 20, 25, or 30 amino acids in length (and optionally up to 50, 40, 30, 25, 20, 15, 10, or 5 amino acids in length). In some embodiments, a linker can be used to space a first moiety from a second, e.g., a targeting moiety from an effector moiety. In some embodiments, for example, a linker can be positioned between a targeting moiety and an effector moiety, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g. polypeptide) from a modulating agent, but sufficiently stable to prevent premature cleavage.

In some embodiments, one or more moieties of a modulating agent described herein are linked with one or more linkers.

As will be known by one of skill in the art, commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of a linker in aqueous solutions by forming hydrogen bonds with water molecules, and therefore reduce unfavorable interactions between a linker and protein moieties.

Rigid linkers are useful to keep a fixed distance between domains and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments.

Examples of linking molecules include a hydrophobic linker, such as a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more components of a modulating agent (e.g. two polypeptides). Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine or other hydrophobic residue. Components of a modulating agent may be linked using charge-based chemistry, such that a positively charged component of a modulating agent is linked to a negative charge of another component or nucleic acid.

In some embodiments, a modulating agent, e.g., fusion molecule, has the capacity to form linkages, e.g., after administration (e.g. to a subject), to other polypeptides, to another moiety as described herein, e.g., an effector molecule, e.g., a nucleic acid, protein, peptide or other molecule, or other agents, e.g., intracellular molecules, such as through covalent bonds or non-covalent bonds. In some embodiments, one or more amino acids on a polypeptide of a modulating agent are capable of linking with a nucleic acid, such as through arginine forming a pseudo-pairing with guanosine or an internucleotide phosphate linkage or an interpolymeric linkage. In some embodiments, a nucleic acid is a DNA such as genomic DNA, RNA such as tRNA or mRNA molecule. In some embodiments, one or more amino acids on a polypeptide are capable of linking with a protein or peptide.

In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

### Tagging or Monitoring Moieties

A modulating agent, e.g., fusion molecule, may further comprise a tagging or monitoring moiety, e.g., to label or monitor the modulating agent, a target component, or an effector function. A person of skill in the art will be aware of many tagging or monitoring moieties compatible with the modulating agents, e.g., fusion molecules, of the disclosure; these include, but are not limited to: affinity tags, solubilization tags, light sensitive tags, fluorescent tags, and other protein tags. A tagging or monitoring moiety may be removable by chemical agents or enzymatic cleavage, such as proteolysis or intein splicing. An affinity tag may be useful to purify a tagged polypeptide using an affinity technique. Some examples include, chitin binding protein (CBP), maltose binding protein (MBP), glutathione-S-transferase (GST), and poly(His) tag. A solubilization tag may be useful to aid recombinant proteins expressed in chaperone-deficient species such as E. coli to assist in the proper folding in proteins and keep them from precipitating. Some examples include thioredoxin (TRX) and poly(NANP). A tagging or monitoring moiety may include a light sensitive tag, e.g., fluorescence. Fluorescent tags are useful for visualization. GFP and its variants are some examples commonly used as fluorescent tags. Protein tags may allow specific enzymatic modifications (such as biotinylation by biotin ligase) or chemical modifications (such as reaction with FlAsH-EDT2 for fluorescence imaging) to occur. Often tagging or monitoring moiety are combined, in order to connect proteins to multiple other components. A tagging or monitoring moiety may also be removed by specific proteolysis or enzymatic cleavage (e.g. by TEV protease, Thrombin, Factor Xa or Enteropeptidase).

In some embodiments, a tagging or monitoring moiety may be a small molecule, peptide, protein (including, e.g. protein fragment, antibody, antibody fragment, etc), nucleic acid, nanoparticle, aptamer, or other agent or portion thereof.

### Cleavable Moieties

In some embodiments, a modulating agent, e.g., fusion molecule, comprises a moiety (e.g., a targeting, effector, or tagging or monitoring moiety) that may be cleaved from a polypeptide portion of the modulating agent (e.g., after administration) by specific proteolysis or enzymatic cleavage (e.g., by TEV protease, Thrombin, Factor Xa or Enteropeptidase).

### Membrane Translocating Moieties

In some embodiments, a modulating agent, e.g., fusion molecule, of the present disclosure further comprises a membrane translocating polypeptide, e.g., linked to moiety (e.g., the targeting moiety) such as through covalent bonds or non-covalent bonds or a linker as described herein. In some embodiments, a modulating agent, e.g., fusion molecule, comprises a moiety linked to a membrane translocating moiety through a peptide bond. In some embodiments, an amino terminal of a modulating agent, e.g., fusion molecule, is linked to membrane translocating moiety, such as through a peptide bond with an optional linker. In some embodiments, a carboxyl terminal of a modulating agent, e.g., fusion molecule, is linked to a membrane translocating moiety as described herein.

In some embodiments, one or more amino acids of a membrane translocating polypeptide are linked with another moiety, such as through disulfide bonds between cysteine side chains, hydrogen bonding, or any other another moiety may be a ligand or antibody to target a composition to a specific cell expressing a particular receptor. For example, in some embodiments, a chemotherapeutic agent, such as topotecan a topoisomerase inhibitor, is linked to one end of a polypeptide and a ligand or antibody is linked to another end of a polypeptide to target a composition to a specific cell or tissue. In some embodiments, other moieties are both effectors with biological activity.

In some embodiments, a plurality of membrane translocating polypeptides, either the same or different membrane translocating polypeptides, are linked to a single modulating agent. Polypeptides may act as a coating that surrounds a modulating agent and aids in its membrane penetration.

In some embodiments, a modulating agent, e.g., fusion molecule, of the present disclosure may comprise a membrane translocating polypeptide linked to a targeting moiety and/or an effector moiety on one or both ends and another separate moiety may be linked to another site on a polypeptide. In some embodiments, upon administration, a modulating agent, e.g., fusion molecule, penetrates a cell membrane and an effector performs a function. In some embodiments, after an effector performs a function, ubiquitin targets the modulating agent, e.g., fusion molecule, for degradation. In some embodiments, upon administration, modulating agent, e.g., fusion molecule, may target a non-CTCF genomic sequence (e.g., an ncRNA such as an eRNA) to modulate transcription of a gene.

In some embodiments, modulating agent, e.g., fusion molecule, provided by the present disclosure may comprise a membrane translocating polypeptide linked to a targeting moiety and/or an effector moiety through covalent bonds and another optional moiety (e.g., a targeting moiety and/or an effector moiety) linked to nucleic acids in a polypeptide. In some embodiments, for example, a protein synthesis inhibitor is covalently linked to a modulating agent, e.g., fusion molecule, and an siRNA or other target specific nucleic acid is hybridized to nucleic acids in the modulating agent. Upon administration, an siRNA targets a modulating agent, e.g., fusion molecule, to an mRNA transcript and a protein synthesis inhibitor and siRNA act to inhibit expression of an mRNA.

Membrane translocating polypeptides as described herein can be linked to another moiety by employing standard ligation techniques, such as those described herein or known in the art to link polypeptides.

### Pharmacoagent Moieties

In some embodiments, a modulating agent further comprises a pharmacoagent moiety. In some embodiments, such a modulating agent, e.g., fusion molecule, may have undesirable pharmacokinetic or pharmacodynamics (PK/PD) parameter. Linking a pharmacoagent moiety to a targeting moiety and/or an effector moiety may improve at least one PK/PD parameter, such as targeting, absorption, and transport of the pharmacoagent, or reduce at least one undesirable PK/PD parameter, such as diffusion to off-target sites, and toxic metabolism. For example, linking a pharmacoagent moiety to a targeting moiety and/or an effector moiety as described herein to an agent with poor targeting/transport, e.g., doxorubicin, beta-lactams such as penicillin, improves its specificity.

### Small Molecules

As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid. In some embodiments, a modulating agent is or comprises a small molecule (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent. Those of ordinary skill in the art, reading the present disclosure, will appreciate that certain small molecule compounds described herein may be provided and/or utilized in any of a variety of forms such as, for example, crystal forms, salt forms, protected forms, pro-drug forms, ester forms, isomeric forms (e.g., optical and/or structural isomers), isotopic forms, etc. Those of skill in the art will appreciate that certain small molecule compounds have structures that can exist in one or more steroisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form. Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms. Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for 14N; ¹⁷O or ¹⁸O for 16O; ³⁶Cl for XXC; ¹⁸F for XXF; 131I for XXXI; etc). In some embodiments, such a small molecule may be utilized in accordance with the present disclosure in one or more isotopically modified forms, or mixtures thereof. In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form. In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

In some embodiments, a modulating agent, e.g., fusion molecule, comprises one or more small molecules.

In some embodiments, a modulating agent (e.g., a targeting, effector, and/or other moiety thereof) comprises a small molecule that intercalates into a nucleic acid structure, e.g., at a specific site.

In some embodiments, a modulating agent comprises a small molecule pharmacoagent.

In some embodiments, a modulating agent comprises a small molecule that alters one or more DNA methylation sites, e.g., mutates methylated cysteine to thymine, within an anchor sequence-mediated conjunction. For example, bisulfite compounds, e.g., sodium bisulfite, ammonium bisulfite, or other bisulfite salts, may be used to alter one or more DNA methylation sites, e.g., altering a nucleotide sequence from a cysteine to a thymine.

In some embodiments, a small molecule may include, but not be limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds. Small molecules may include, but are not limited to, a neurotransmitter, a hormone, a drug, a toxin, a viral or microbial particle, a synthetic molecule, and agonists or antagonists.

Examples of suitable small molecules include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Some examples of small molecules may include, but are not limited to, prion drugs such as tacrolimus, ubiquitin ligase or HECT ligase inhibitors such as heclin, histone modifying drugs such as sodium butyrate, enzymatic inhibitors such as 5-aza-cytidine, anthracyclines such as doxorubicin, beta-lactams such as penicillin, anti-bacterials, chemotherapy agents, anti-virals, modulators from other organisms such as VP64, and drugs with insufficient bioavailability such as chemotherapeutics with deficient pharmacokinetics.

In some embodiments, a small molecule is an epigenetic modifying agent, for example such as those described in de Groote et al. Nuc. Acids Res. (2012):1-18. Exemplary small molecule epigenetic modifying agents are described, e.g., in Lu et al. J. Biomolecular Screening 17.5(2012):555-71, e.g., at Table 1 or 2, incorporated herein by reference. In some embodiments, an epigenetic modifying agent comprises vorinostat, romidepsin. In some embodiments, an epigenetic modifying agent comprises an inhibitor of class I, II, III, and/or IV histone deacetylase (HDAC). In some embodiments, an epigenetic modifying agent comprises an activator of SirTI. In some embodiments, an epigenetic modifying agent comprises Garcinol, Lys-CoA, C646, (+)-JQI, I-BET, BICI, MS120, DZNep, UNC0321, EPZ004777, AZ505, AMI-I, pyrazole amide 7b, benzo[d]imidazole 17b, acylated dapsone derivative (e.g., PRMT1), methylstat, 4,4′-dicarboxy-2,2′-bipyridine, SID 85736331, hydroxamate analog 8, tanylcypromie, bisguanidine and biguanide polyamine analogs, UNC669, Vidaza, decitabine, sodium phenyl butyrate (SDB), lipoic acid (LA), quercetin, valproic acid, hydralazine, bactrim, green tea extract (e.g., epigallocatechin gallate (EGCG)), curcumin, sulforphane and/or allicin/diallyl disulfide. In some embodiments, an epigenetic modifying agent inhibits DNA methylation, e.g., is an inhibitor of DNA methyltransferase (e.g., is 5-azacitidine and/or decitabine). In some embodiments, an epigenetic modifying agent modifies histone modification, e.g., histone acetylation, histone methylation, histone sumoylation, and/or histone phosphorylation. In some embodiments, an epigenetic modifying agent is an inhibitor of a histone deacetylase (e.g., is vorinostat and/or trichostatin A).

In some embodiments, a small molecule is a pharmaceutically active agent. In some embodiments, a small molecule is an inhibitor of a metabolic activity or component. Useful classes of pharmaceutically active agents include, but are not limited to, antibiotics, anti-inflammatory drugs, angiogenic or vasoactive agents, growth factors and/or chemotherapeutic agents. One or a combination of molecules from categories and examples as described herein or from (Orme-Johnson 2007, Methods Cell Biol. 2007; 80:813-26) can be used. In some embodiments, the present disclosure provides compositions comprising one or more antibiotics, anti-inflammatory drugs, angiogenic or vasoactive agents, growth factors and/or chemotherapeutic agents.

In some embodiments, a modulating agent, e.g., a fusion molecule, comprises a small molecule moiety (e.g., a peptidomimetic or a small organic molecule with a molecular weight of less than 2000 daltons), a peptide or polypeptide (e.g., an antibody or antigen-binding fragment thereof), a nucleic acid (e.g., siRNA, mRNA, RNA, DNA, modified DNA or RNA, antisense DNA oligonucleotides, an antisense RNA, a ribozyme, a therapeutic mRNA encoding a protein), a nanoparticle, an aptamer, or pharmacoagent with poor PK/PD.

### Compositions: Methods of Making, Formulation, Delivery, and Administration

The present disclosure, among other things, provide compositions that comprise or deliver a modulating agent, e.g., fusion molecule. In some embodiments, a modulating agent, e.g., fusion molecule, that comprises a polypeptide moiety or entity may be provided via a composition that includes the fusion molecule, e.g., polypeptide moiety or entity, or alternatively via a composition that includes a nucleic acid encoding the fusion molecule, e.g., polypeptide moiety or entity, and associated with sufficient other sequences to achieve expression of the fusion molecule, e.g., polypeptide moiety or entity, in a system of interest (e.g., in a particular cell, tissue, organism, etc).

In some embodiments, a provided composition may be a pharmaceutical composition whose active ingredient comprises or delivers a modulating agent, e.g., fusion molecule, as described herein and is provided in combination with one or more pharmaceutically acceptable excipients, optionally formulated for administration to a subject (e.g., to a cell, tissue, or other site thereof).

Thus, in some embodiments, the present disclosure provides compositions comprising a modulating agent (e.g., fusion molecule), or a production intermediate thereof. In some particular embodiments, the present disclosure provides compositions of nucleic acids that encode a modulating agent (e.g., fusion molecule) or polypeptide portion thereof. In some such embodiments, provided nucleic acids may be or include DNA, RNA, or any other nucleic acid moiety or entity as described herein, and may be prepared by any technology described herein or otherwise available in the art (e.g., synthesis, cloning, amplification, in vitro or in vivo transcription, etc). In some embodiments, provided nucleic acids that encode a modulating agent (e.g., fusion molecule) or polypeptide portion thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc).

In some embodiments, a modulating agent (e.g., fusion molecule) is or comprises a vector, e.g., a viral vector, comprising one or more nucleic acids encoding one or more components of a modulating agent (e.g., fusion molecule) as described herein.

Nucleic acids as described herein or nucleic acids encoding a protein described herein, may be incorporated into a vector. Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. An expression vector may be provided to a cell in the form of a viral vector. Viral vector technology is well known in the art, and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Expression of natural or synthetic nucleic acids is typically achieved by operably linking a nucleic acid encoding the gene of interest to a promoter, and incorporating the construct into an expression vector. Vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, may regulate frequency of transcriptional initiation. Typically, these sequences are located in a region 30-110 bp upstream of a transcription start site, although a number of promoters have recently been shown to contain functional elements downstream of transcription start sites as well. Spacing between promoter elements frequently is flexible, so that promoter function is preserved when elements are inverted or moved relative to one another. In a thymidine kinase (tk) promoter, spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. In some embodiments of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, an actin promoter, a myosin promoter, a hemoglobin promoter, and a creatine kinase promoter. The present disclosure should not interpreted to be limited to use of any particular promoter or category of promoters (e.g. constitutive promoters). For example, in some embodiments, inducible promoters are contemplated as part of the present disclosure. In some embodiments, use of an inducible promoter provides a molecular switch capable of turning on expression of a polynucleotide sequence to which it is operatively linked, when such expression is desired. In some embodiments, use of an inducible promoter provides a molecular switch capable of turning off expression when expression is not desired. Examples of inducible promoters include, but are not limited to a metallothionine promoter, a glucocorticoid promoter, a progesterone promoter, and a tetracycline promoter.

In some embodiments, an expression vector to be introduced can also contain either a selectable marker gene or a reporter gene or both to facilitate identification and selection of expressing cells from the population of cells sought to be transfected or infected through viral vectors. In some aspects, a selectable marker may be carried on a separate piece of DNA and used in a co-transfection procedure. Both selectable markers and reporter genes may be flanked with appropriate transcriptional control sequences to enable expression in the host cells. Useful selectable markers may include, for example, antibiotic-resistance genes, such as neo, etc.

In some embodiments, reporter genes may be used for identifying potentially transfected cells and/or for evaluating the functionality of transcriptional control sequences. In general, a reporter gene is a gene that is not present in or expressed by a recipient source (of a reporter gene) and that encodes a polypeptide whose expression is manifested by some easily detectable property, e.g., enzymatic activity or visualizable fluorescence. Expression of a reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, a construct with a minimal 5′ flanking region that shows highest level of expression of reporter gene is identified as a promoter. Such promoter regions may be linked to a reporter gene and used to evaluate agents for ability to modulate promoter-driven transcription.

In various embodiments compositions described herein (e.g., modulating agents, e.g., fusion molecules) are pharmaceutical compositions. In some embodiments, compositions (e.g. pharmaceutical compositions) described herein may be formulated for delivery to a cell and/or to a subject via any route of administration. Modes of administration to a subject may include injection, infusion, inhalation, intranasal, intraocular, topical delivery, intercannular delivery, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), or local (e.g., local application on the skin, intravitreal injection). In some embodiments, one or more compositions is administered systemically. In some embodiments, administration is non-parenteral and a therapeutic is a parenteral therapeutic. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be a single dose. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “pharmaceutical composition” refers to an active agent (e.g., fusion molecule), formulated together with one or more pharmaceutically acceptable carriers (e.g., pharmaceutically acceptable carriers known to those of skill in the art). In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and/or to other mucosal surfaces.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In some embodiments, for example, materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt”, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

In various embodiments, the present disclosure provides pharmaceutical compositions described herein with a pharmaceutically acceptable excipient. Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutical preparations may be made following conventional techniques of pharmacy involving milling, mixing, granulation, and compressing, when necessary, for tablet forms; or milling, mixing and filling for hard gelatin capsule forms. When a liquid carrier is used, a preparation can be in the form of a syrup, elixir, emulsion or an aqueous or non-aqueous solution or suspension. Such a liquid formulation may be administered directly per os.

Pharmaceutical compositions according to the present disclosure may be delivered in a therapeutically effective amount. A precise therapeutically effective amount is an amount of a composition that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

In some aspects, the present disclosure provides methods of delivering a therapeutic comprising administering a composition as described herein to a subject, wherein a genomic complex modulating agent is a therapeutic and/or wherein delivery of a therapeutic causes changes in gene expression relative to gene expression in absence of a therapeutic.

Methods as provided in various embodiments herein may be utilized in any some aspects delineated herein. In some embodiments, one or more compositions is/are targeted to specific cells, or one or more specific tissues.

For example, in some embodiments one or more compositions is/are targeted to epithelial, connective, muscular, and/or nervous tissue or cells. In some embodiments a composition is targeted to a cell or tissue of a particular organ system, e.g., cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage); and/or combinations thereof. In some embodiments, a composition of the present disclosure crosses a blood-brain-barrier, a placental membrane, or a blood-testis barrier.

In some embodiments, a composition as provided herein is administered systemically.

In some embodiments, administration is non-parenteral and a therapeutic is a parenteral therapeutic.

In some embodiments, a composition of the present disclosure has improved PK/PD, e.g., increased pharmacokinetics or pharmacodynamics, such as improved targeting, absorption, or transport (e.g., at least 5%, 10%, 15%, 20%, 30%, 40%, 50%, 60%, 75%, 80%, 90% improved or more) as compared to a therapeutic alone. In some embodiments, a composition has reduced undesirable effects, such as reduced diffusion to a nontarget location, off-target activity, or toxic metabolism, as compared to a therapeutic alone (e.g., at least 5%, 10%, 15%, 20%, 30%, 40%, 50%, 60%, 75%, 80%, 90% or more reduced, as compared to a therapeutic alone). In some embodiments, a composition increases efficacy and/or decreases toxicity of a therapeutic (e.g., at least 5%, 10%, 15%, 20%, 30%, 40%, 50%, 60%, 75%, 80%, 90% or more) as compared to a therapeutic alone.

Pharmaceutical compositions described herein may be formulated for example including a carrier, such as a pharmaceutical carrier and/or a polymeric carrier, e.g., a liposome or vesicle, and delivered by known methods to a subject in need thereof (e.g., a human or non-human agricultural or domestic animal, e.g., cattle, dog, cat, horse, poultry). Such methods include transfection (e.g., lipid-mediated, cationic polymers, calcium phosphate); electroporation or other methods of membrane disruption (e.g., nucleofection) and viral delivery (e.g., lentivirus, retrovirus, adenovirus, AAV). Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

Pharmaceutical uses of the present disclosure may include compositions (e.g. modulating agents, e.g., fusion molecules) as described herein. In some aspects, a system for pharmaceutical use comprises: a protein comprising a first polypeptide domain, e.g., a Cas or modified Cas protein, and a second polypeptide domain, e.g., a polypeptide having DNA methyltransferase activity or associated with demethylation or deaminase activity, in combination with at least one guide RNA (gRNA) or antisense DNA oligonucleotide that targets an ncRNA, such as an eRNA. A system is effective to alter, in at least a human cell, a genomic complex or transcription complex, e.g., a target anchor sequence-mediated conjunction.

In some embodiments, pharmaceutical compositions of the present disclosure comprise a zinc finger nuclease (ZFN), or a mRNA encoding a ZFN, that targets (e.g., cleaves) an ncRNA, such as an eRNA.

In some aspects, a system for pharmaceutical use comprises a composition that binds an ncRNA, such as an eRNA, and alters formation of a genomic complex or transcription complex comprising the ncRNA (e.g., eRNA), e.g., an anchor sequence-mediated conjunction, wherein such a composition modulates transcription, in a human cell, of a target gene associated with the genomic complex or transcription complex, e.g., anchor sequence-mediated conjunction.

In some aspects, a system for altering, in a human cell, expression of a target gene, comprises a targeting moiety (e.g., a gRNA, a membrane translocating polypeptide) that associates with an an ncRNA, such as an eRNA, associated with a target gene, and an effector moiety (e.g. an enzyme, e.g., a nuclease or deactivated nuclease (e.g., a Cas9, dCas9), a methylase, a de-methylase, a deaminase) operably linked to the targeting moiety, wherein the system is effective to alter (e.g., decrease) expression of the target gene. The targeting moiety and effector moiety may be different and separate (e.g., comprised in different physical portions of a fusion molecule) moieties. A targeting moiety and an effector moiety may be linked, e.g., covalently, e.g., by a linker. In some embodiments, a system comprises a synthetic polypeptide comprising a targeting moiety and an effector moiety. In some embodiments, a system comprises a nucleic acid vector or vectors encoding at least one of a targeting moiety and an effector moiety.

In some aspects, pharmaceutical compositions may comprise a modulating agent, e.g., fusion molecule, that binds an an ncRNA, such as an eRNA, and alters, e.g., decreases, formation of an genomic or transcription complex, e.g., an anchor sequence-mediated conjunction, wherein the modulating agent modulates transcription, in a human cell, of a target gene associated with the genomic or transcription complex, e.g., anchor sequence-mediated conjunction. In some embodiments, a modulating agent, e.g., fusion molecule, disrupts formation of an genomic or transcription complex, e.g., anchor sequence-mediated conjunction (e.g., decreases affinity of an anchor sequence to a conjunction nucleating molecule, e.g., at least 10%, 15%, 20%, 25%, 30%, 35%, 40%, 45%, 50%, 55%, 60%, 65%, 70%, 75%, 80%, 85%, 90%, 95%, or more).

In some embodiments, administration of compositions described herein improves at least one pharmacokinetic or pharmacodynamic parameter of at least one component of the composition (e.g. a pharmacoagent), such as targeting, absorption, and transport, as compared to another moiety alone, or reduces at least one toxicokinetic parameter, such as diffusion to non-target location, off-target activity, and toxic metabolism, as compared to another moiety alone (e.g., by at least 5%, 10%, 20%, 25%, 30%, 40%, 50%, 60%, 70%, 80% or more). In some embodiments, administration of compositions of the present disclosure increases a therapeutic range of at least one component of a modulating agent (e.g., by at least 5%, 10%, 20%, 25%, 30%, 40%, 50%, 60%, 70%, 80% or more). In some embodiments, administration of compositions provided herein reduces a minimum effective dose, as compared to another moiety alone (e.g., by at least 5%, 10%, 20%, 25%, 30%, 40%, 50%, 60%, 70%, 80% or more). In some embodiments, administration of compositions provided increases a maximum tolerated dose, as compared to a modulating agent alone (e.g., by at least 5%, 10%, 20%, 25%, 30%, 40%, 50%, 60%, 70%, 80% or more). In some embodiments, administration of compositions provided herein increases efficacy or decreases toxicity of a therapeutic, such as non-parenteral administration of a parenteral therapeutic. In some embodiments, administration of compositions provided herein increases a therapeutic range of a modulating agent while decreasing toxicity, as compared to a modulating agent alone (e.g., by at least 5%, 10%, 20%, 25%, 30%, 40%, 50%, 60%, 70%, 80% or more).

In some embodiments, a modulating agent, e.g., fusion molecule, comprises or is a protein and may thus be produced by methods of making proteins. As will be appreciated by one of skill, methods of making proteins or polypeptides (which may be included in modulating agents as described herein) are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

A protein or polypeptide of compositions of the present disclosure can be biochemically synthesized by employing standard solid phase techniques. Such methods include exclusive solid phase synthesis, partial solid phase synthesis methods, fragment condensation, classical solution synthesis. These methods can be used when a peptide is relatively short (e.g., 10 kDa) and/or when it cannot be produced by recombinant techniques (i.e., not encoded by a nucleic acid sequence) and therefore involves different chemistry.

Solid phase synthesis procedures are well known in the art and further described by John Morrow Stewart and Janis Dillaha Young, Solid Phase Peptide Syntheses, 2nd Ed., Pierce Chemical Company, 1984; and Coin, I., et al., Nature Protocols, 2:3247-3256, 2007.

For longer peptides, recombinant methods may be used. Methods of making a recombinant therapeutic polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Exemplary methods for producing a therapeutic pharmaceutical protein or polypeptide involve expression in mammalian cells, although recombinant proteins can also be produced using insect cells, yeast, bacteria, or other cells under control of appropriate promoters. Mammalian expression vectors may comprise nontranscribed elements such as an origin of replication, a suitable promoter, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In cases where large amounts of the protein or polypeptide are desired, it can be generated using techniques such as described by Brian Bray, Nature Reviews Drug Discovery, 2:587-593, 2003; and Weissbach & Weissbach, 1988, Methods for Plant Molecular Biology, Academic Press, NY, Section VIII, pp 421-463.

Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein. Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012). Proteins comprise one or more amino acids. Amino acids include any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

In some aspects, the present disclosure provides a modulating agent, e.g., a fusion molecule, comprising a targeting moiety that binds an ncRNA, such as an eRNA, and alters, e.g., decreases or increases, formation of a genomic or transcription complex, e.g., an anchor sequence-mediated conjunction (e.g., decreases the level of the complex by at least 10%, 15%, 20%, 25%, 30%, 35%, 40%, 45%, 50%, 55%, 60%, 65%, 70%, 75%, 80%, 85%, 90%, 95%, or more).

In some aspects, a pharmaceutical composition includes a Cas protein and at least one guide RNA (gRNA) that targets a Cas protein to an ncRNA, such as an eRNA.

In some embodiments, a gRNA is administered in combination with a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. Choice of nuclease and gRNA(s) is determined by whether a targeted mutation is a deletion, substitution, or addition of nucleotides, e.g., a deletion, substitution, or addition of nucleotides to an ncRNA, such as an eRNA. For example, in some embodiments, one gRNA is administered, e.g., to produce an inactivating indel mutation in an ncRNA, such as an eRNA, e.g., one gRNA is administered in combination with a nuclease, e.g., wtCas9.

In some aspects, the present disclosure provides a composition comprising a nucleic acid or combination of nucleic acids that when administered to a subject in need thereof introduce a site specific alteration (e.g., insertion, deletion (e.g., knockout), translocation, inversion, single point mutation) in a target sequence of a target genomic complex/transcription complex or of a component of a target genomic complex or transcription complex, e.g., an ncRNA, eRNA, thereby modulating gene expression in a subject.

### Uses

Technologies provided herein achieve modulation of structure and/or function of genomic complexes or transcription complexes. Among other things, in some embodiments such provided technologies achieve modulation of gene expression and, for example, enable breadth over controlling gene activity, delivery, and penetrance, e.g., in a cell. In some embodiments, a cell is a mammalian cell. In some embodiments, a cell is a somatic cell. In some embodiments, a cell is a primary cell. For example, in some embodiments, a cell is a mammalian somatic cell. In some embodiments, a mammalian somatic cell is a primary cell. In some embodiments, a mammalian somatic cell is a non-embryonic cell.

In some embodiments, provided methods comprise a step of: delivering modulating agent (e.g., fusion molecule) to a cell. In some embodiments, a step of delivering is performed ex vivo. In some embodiments, methods further comprise, prior to the step of delivering, a step of removing a cell (e.g., a mammalian cell) from a subject. In some embodiments, methods further comprise, after the step of delivering, a step of (b) administering cells (e.g., mammalian cells) to a subject. In some embodiments, the step of delivering comprises administering a composition comprising a modulating agent (e.g., fusion molecule) to a subject. In some embodiments, a subject has a disease or condition.

In some embodiments, the step of delivering comprises delivery across a cell membrane. In some embodiments, provided methods comprise a step of (a) substituting, adding, or deleting one or more nucleotides of an ncRNA, such as an eRNA, within a cell, e.g., a mammalian somatic cell.

In some embodiments, the step of substituting, adding, or deleting is performed in vivo. In some embodiments, the step of substituting, adding, or deleting is performed ex vivo.

In some embodiments, provided methods comprise a step of delivering a mammalian somatic cell to a subject having a disease or condition, wherein one or more nucleotides of an ncRNA, such as an eRNA, within a mammalian somatic cell has been substituted, added, or deleted.

In some embodiments, provided methods comprise a step of: (a) administering somatic mammalian cells to a subject, wherein somatic mammalian cells were obtained from a subject, and modulating agent (e.g., fusion molecule) as described herein had been delivered ex vivo to somatic mammalian cells.

In some embodiments, indications that affect any one of blood, liver, immune system, neuronal system, etc. or combinations thereof may be treated by modulating gene expression through altering a genomic complex or transcription complex, e.g., an anchor sequence-mediated conjunction, in a mammalian subject.

In some aspects, provided methods comprise altering gene expression or altering a genomic complex or transcription complex, e.g., an anchor sequence-mediated conjunction, in a mammalian subject. Methods may include administering to a subject (separately or in a single pharmaceutical composition): a protein comprising a first polypeptide domain that comprises a Cas or modified Cas protein and a second polypeptide domain that comprises a polypeptide having DNA methyltransferase activity (or associated with demethylation or deaminase activity), or a nucleic acid encoding a protein comprising a first polypeptide domain that comprises a Cas or modified Cas protein and a second polypeptide domain that comprises a polypeptide having DNA methyltransferase activity (or associated with demethylation or deaminase activity), and at least one guide RNA (gRNA) that targets an ncRNA, such as an eRNA. In some embodiments, a gRNA targets a component of a genomic complex or transcription complex, such as an ncRNA or eRNA.

Methods and compositions as provided herein may treat disease by stably or transiently altering (e.g., decreasing) a genomic complex or transcription complex, e.g., an anchor sequence-mediated conjunction, or modulating transcription of a nucleic acid sequence. In some embodiments, chromatin structure or topology of a genomic complex or transcription complex, e.g., an anchor sequence-mediated conjunction, is altered to result in a stable modulation of transcription, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some other embodiments, chromatin structure or topology of a genomic complex or transcription complex, e.g., an anchor sequence-mediated conjunction, is altered to result in a transient modulation of transcription, such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some aspects, methods provided by the present disclosure may comprise modifying expression of a target gene, comprising administering to a cell, tissue or subject a modulating agent, e.g., fusion molecule, as described herein.

In some aspects, the present disclosure provides methods of modifying expression of a target gene, comprising altering a genomic complex or transcription complex, e.g., an anchor sequence-mediated conjunction, associated with a target gene, wherein an alteration modulates transcription of a target gene.

In some embodiments, provided technologies may comprise inducibly altering a genomic complex or transcription complex, e.g., an anchor sequence-mediated conjunction, or portion thereof (e.g., ncRNA, e.g., eRNA). Use of an inducible alteration to an anchor-mediated conjunction or other component of a genomic complex (e.g. ncRNA, e.g., eRNA) provides a molecular switch. In some embodiments, a molecular switch is capable of turning on an alteration when desired. In some embodiments, a molecular switch is capable of turning off an alteration when it is not desired. In some embodiments, a molecular switch is capable of both turning on and turning off an alteration, as desired. Examples of systems used for inducing alterations include, but are not limited to an inducible targeting moiety based on a prokaryotic operon, e.g., the lac operon, transposon Tn10, tetracycline operon, and the like, and an inducible targeting moiety based on a eukaryotic signaling pathway, e.g. steroid receptor-based expression systems, e.g., the estrogen receptor or progesterone-based expression system, the metallothionein-based expression system, the ecdysone-based expression system.

In some embodiments, cells or tissue may be excised from a subject and gene expression, e.g., endogenous or exogenous gene expression, may be altered ex vivo prior to transplantation of cells or tissues back into a subject. Any cell or tissue may be excised and used for re-transplantation. Some examples of cells and tissues include, but are not limited to, stem cells, adipocytes, immune cells, myocytes, bone marrow derived cells, cells from the kidney capsule, fibroblasts, endothelial cells, and hepatocytes. In some embodiments, for example, adipose tissue from a patient may be altered ex vivo to increase energy production and lipid utilization. Modified adipose cells are returned to a patient from whom they were excised and act as “furnaces,” e.g., they uptake lipids from circulation and use them for energy production.

The present disclosure also provides methods of delivering a composition described herein to a subject. In some embodiments, a composition is delivered across a cellular membrane, e.g., a plasma membrane, a nuclear membrane, an organellar membrane. Current polymeric delivery technologies increase endocytic rates in certain cell types, usually cells that preferentially utilize endocytosis, such as macrophages and other cell types that rely on calcium influx to trigger endocytosis. Without being bound by any particular theory, a composition described herein is believed to aid movement of a composition across membranes typically inaccessible by most agents.

In some aspects, a kit is described that includes: (a) a nucleic acid encoding a protein comprising a first polypeptide domain that comprises a Cas or modified Cas protein and a second polypeptide domain, e.g., a polypeptide having DNA methyltransferase activity or associated with demethylation or deaminase activity, and (b) at least one guide RNA (gRNA) for targeting a protein to an anchor sequence of a target anchor sequence-mediated conjunction in a target cell. In some embodiments, a nucleic acid encoding a protein and a gRNA are in the same vector, e.g., a plasmid, an AAV vector, an AAV9 vector. In some embodiments, a nucleic acid encoding a protein and a gRNA are in separate vectors.

### Modulating Gene Expression

As will be appreciated by one of skill in the art, particular genes are known to be associated with complexes and in many cases effect of a given genomic complex or transcription complex on gene expression is known. Thus, in some embodiments, as described herein, complex inhibition inhibits expression of an associated gene. In some embodiments, as described herein, complex inhibition promotes expression of an associated gene.

In some embodiments, transcription of a nucleic acid sequence is modulated, e.g., transcription of a target nucleic acid sequence, as compared with a reference value, e.g., transcription of a target sequence in absence of an altered genomic complex or transcription complex, e.g., anchor sequence-mediated conjunction.

In some embodiments, provided are technologies for modulating expression of a gene associated with a genomic complex or transcription complex, e.g., an anchor sequence-mediated conjunction, which conjunction comprises a first anchor sequence and a second anchor sequence. A gene that is associated with an anchor sequence-mediated conjunction may be at least partially within a conjunction (that is, situated sequence-wise between first and second anchor sequences), or it may be external to a conjunction in that it is not situated sequence-wise between a first and second anchor sequences, but is located on the same chromosome and in sufficient proximity to at least a first or a second anchor sequence such that its expression can be modulated by controlling the topology of the anchor sequence-mediated conjunction. Those of ordinary skill in the art will understand that distance in three-dimensional space between two elements (e.g., between the gene and the anchor sequence-mediated conjunction) may, in some embodiments, be more relevant than distance in terms of basepairs. In some embodiments, an external but associated gene is located within 2 Mb, within 1.9 Mb, within 1.8 Mb, within 1.7 Mb, within 1.6 Mb, within 1.5 Mb, within 1.4 Mb, with 1.3 Mb, within 1.3 Mb, within 1.2 Mb, within 1.1 Mb, within 1 Mb, within 900 kb, within 800 kb, within 700 kb, within 500 kb, within 400 kb, within 300 kb, within 200 kb, within 100 kb, within 50 kb, within 20 kb, within 10 kb, or within 5 kb of the first or second anchor sequence.

In some embodiments, modulating expression of a gene comprises altering accessibility of a transcriptional control sequence to a gene. A transcriptional control sequence, whether internal or external to an anchor sequence-mediated conjunction, can be an enhancing sequence or a silencing (or repressive) sequence.

For example, in some embodiments, methods are provided for modulating expression of a gene within an anchor sequence-mediated conjunction comprising a step of: contacting the first and/or second anchor sequence with a modulating agent as described herein. In some embodiments, an anchor sequence-mediated conjunction comprises at least one transcriptional control sequence that is “internal” to a conjunction in that it is at least partially located sequence-wise between first and second anchor sequences. Thus, in some embodiments, both a gene whose expression is to be modulated (the “target gene”) and a transcriptional control sequence are within an anchor sequence-mediated conjunction.

In some embodiments, a gene is separated from an internal transcriptional control sequence by at least 300, at least 400, at least 500, at least 600, at least 700, at least 800, or at least 900 base pairs. In some embodiments, a gene is separated from an internal transcriptional control sequence by at least 1.0, at least 1.2, at least 1.4, at least 1.6, or at least 1.8 kb. In some embodiments, a gene is separated from an internal transcriptional control sequence by at least 2 kb, at least 3 kb, at least 4 kb, at least 5 kb, at least 6 kb, at least 7 kb, at least 8 kb, at least 9 kb, or at least 10 kb. In some embodiments, a gene is separated from an internal transcriptional control sequence by at least 20 kb, at least 30 kb, at least 40 kb, at least 50 kb, at least 60 kb, at least 70 kb, at least 80 kb, at least 90 kb, or at least 100 kb. In some embodiments, a gene is separated from an internal transcriptional control sequence by at least 150 kb, at least 200 kb, at least 250 kb, at least 300 kb, at least 350 kb, at least 400 kb, at least 450 kb, or at least 500 kb. In some embodiments, the gene is separated from an internal transcriptional control sequence by at least 600 kb, at least 700 kb, at least 800 kb, at least 900 kb, or at least 1 Mb.

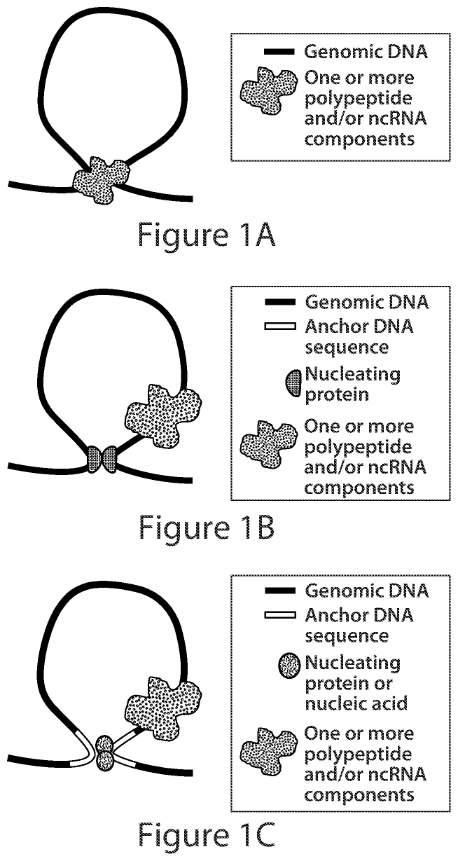

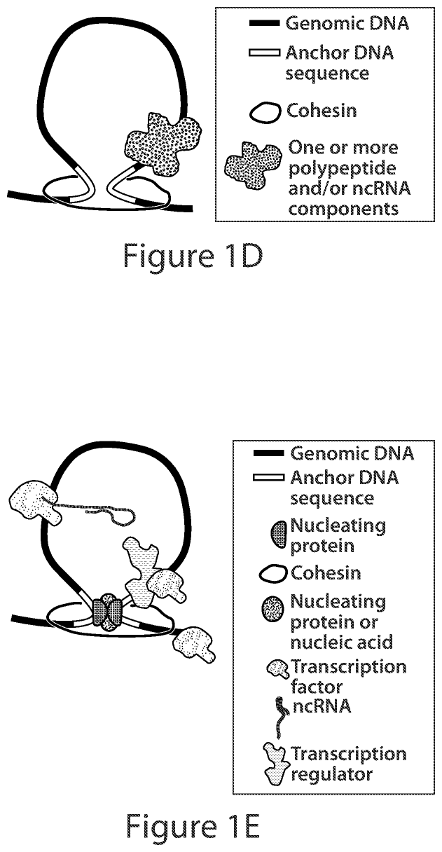

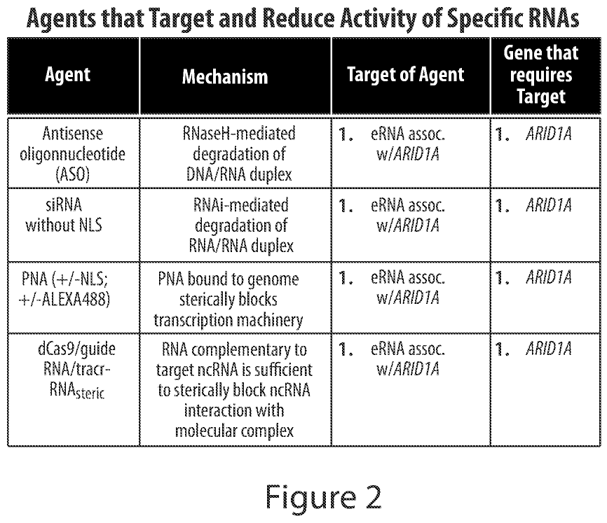

In some embodiments, an anchor sequence-mediated conjunction comprises at least one transcriptional control sequence that is “external” to the conjunction in that it is not located sequence-wise between first and second anchor sequences. (See, e.g., Types 2, 3, and 4 anchor sequence-mediated conjunctions depicted in FIG. 1.) In some embodiments, a first and/or a second anchor sequence is located within 1 Mb, within 900 kb, within 800 kb, within 700 kb, within 600 kb, within 500 kb, within 450 kb, within 400 kb, within 350 kb, within 300 kb, within 250 kb, within 200 kb, within 180 kb, within 160 kb, within 140 kb, within 120 kb, within 100 kb, within 90 kb, within 80 kb, within 70 kb, within 60 kb, within 50 kb, within 40 kb, within 30 kb, within 20 kb, or within 10 kb of an external transcriptional control sequence. In some embodiments, the first and/or the second anchor sequence is located within 9 kb, within 8 kb, within 7 kb, within 6 kb, within 5 kb, within 4 kb, within 3 kb, within 2 kb, or within 1 kb of an external transcriptional control sequence.

For example, in some embodiments, methods are provided for modulating expression of a gene external to an anchor sequence-mediated conjunction comprising a step of: contacting a first and/or second anchor sequence with a modulating agent as described herein. In some embodiments, an anchor sequence-mediated conjunction comprises at least one internal transcriptional control sequence.

In some embodiments, an anchor sequence-mediated conjunction comprises at least one external transcriptional control sequence.

In some embodiments, a modulating agent comprising a targeting moiety that targets an eRNA associated with a genomic or transcription complex comprising the disease-related gene is administered, thereby treating the disease (e.g., by inhibiting expression of the disease-related gene).

For example, compositions and methods described herein may be used to treat severe congenital neutropenia (SCN). In some embodiments, expression of the ELANE gene, which causes the disease, is inhibited. A targeting moiety is administered to target one or more anchor sequences adjacent to the ELANE gene for alteration and create a repressive complex comprising the ELANE gene.

In some aspects, the present disclosure provides methods of treating SCN with a pharmaceutical composition described herein. In some embodiments, administration of one or more compositions as described herein modulates gene expression of one or more genes, such as inhibiting gene expression of the ELANE gene, to treat SCN.

Compositions and methods described herein may be used to treat sickle cell anemia and beta thalassemia. In some embodiments, expression of the HbF from the HBG genes (shown to restore normal hemoglobin levels) is activated. A targeting moiety is administered to target one or more anchor sequences adjacent in the HBB gene cluster or the HBG genes. In some embodiments, an inhibitory complex comprising the HBB gene cluster is created. In some embodiments, an activation complex comprising the HBG genes is created. Downregulating BCL11A has also been shown to downregulate HBB and upregulate HBG expression. In some embodiments, an inhibitory anchor sequence-mediated conjunction associated with the BCL11A gene cluster is created.

In some aspects, the present disclosure provides methods of treating sickle cell anemia and beta thalassemia with a pharmaceutical composition described herein. In some embodiments, administration of a composition described herein modulates gene expression of one or more genes, such as modulating gene expression from the HBB gene cluster or the HBG genes, to treat SCN.

Compositions and methods described herein may be used to treat MYC-related tumors, e.g., MYC-addicted cancers. In some embodiments, expression of MYC, shown to cause tumors, is inhibited. A targeting moiety is administered to target one or more anchor sequences adjacent in the MYC gene. In some embodiments, an inhibitory complex comprising the MYC gene is created. In some embodiments, MYC expression is decreased by disrupting a MYC-associated anchor sequence-mediated conjunction, e.g., decreased transcription due to conformational changes of DNA previously open to transcription within an anchor sequence-mediated conjunction, e.g., decreased transcription due to conformational changes of the DNA creating additional distance between the MYC gene and an enhancing sequence. In some aspects, the present disclosure provides methods of treating MYC-related tumors with a pharmaceutical composition described herein. In some embodiments, administration of one or more compositions described herein modulates gene expression of one or more genes, such as modulating gene expression from the MYC gene, to treat MYC-related tumors.

Compositions and methods described herein may be used to treat myoclonic epilepsy of infancy (SMEI or Dravet's syndrome). In some embodiments, loss-of-function mutations in Naᵥ1.1, also known as the sodium channel, voltage-gated, type I, alpha subunit (SCN1A), from the SCN1A gene, cause severe Dravet's syndrome. In some embodiments, a modulating agent is administered to target one or more anchor sequences adjacent in the SCN1A gene. In some embodiments, a modulating agent is administered to target one or more anchor sequences adjacent in the SCN3A gene to increase expression of Naᵥ1.3, also known as the sodium channel, voltage-gated, type III, alpha subunit (SCN3A). In some embodiments, a modulating agent is administered to target one or more anchor sequences adjacent in the SCN5A gene to increase expression of Naᵥ1.5, also known as the sodium channel, voltage-gated, type V, alpha subunit (SCN5A). In some embodiments, a modulating agent is administered to target one or more anchor sequences adjacent in the SCN8A gene to increase expression of Naᵥ1.6, also known as the sodium channel, voltage-gated, type VIII, alpha subunit (SCN8A). In some embodiments an activation complex comprising any one of SCN1A, SCN3A, SCN5A, and SCN8A genes is created to increase expression of Naᵥ1.1, Naᵥ1.3, Naᵥ1.5, and Naᵥ1.6, respectively.

In some aspects, the present disclosure provides methods of treating Dravet's syndrome with a pharmaceutical composition described herein. In some embodiments, administration of a composition described herein modulates gene expression of one or more genes, such as modulating gene expression from the SCN1A, SCN3A, SCN5A, and SCN8A genes, to treat Dravet's syndrome. In some embodiments, administration of a composition comprising a membrane translocating polypeptide linked to a GABA agonist to increase GABA activity.

Compositions and methods as described herein may be used to treat familial erythromelalgia. In some embodiments, loss-of-function mutations in Naᵥ1.7, also known as the sodium channel, voltage-gated, type IX, alpha subunit (SCN9A), from the SCN9A gene, cause severe familial erythromelalgia. In some embodiments, a targeting moiety is administered to target one or more anchor sequences adjacent in the SCN9A gene. In some embodiments an activation complex comprising the SCN9A gene is created to increase expression of Naᵥ1.7.

In some aspects, the present disclosure provides methods of treating familial erythromelalgia with pharmaceutical compositions provided herein. In some embodiments, administration of compositions described herein may modulate gene expression of one or more genes, such as modulating gene expression from the SCN9A gene. In some embodiments, modulation of the SCN9A gene treats familial erythromelalgia. Methods provided herein may also improve existing therapeutics to increase bioavailability and/or reduce toxicokinetics.

Thus, among other things, the present application provides technologies for modulating gene expression by modulating genomic complexes as described herein.

In some embodiments, modulation may include inducing disruption or formation of insulated neighborhoods. In some embodiments, modulating insulated neighborhoods affects transcription by interfering with formation/reducing frequency of assembly/inducing dissociation of a genomic complex, i.e. a cellular complex responsible for mediating any regulatory effect(s) that insulated neighborhoods have on gene transcription.

In some aspects, the present disclosure provides methods that disrupt one or more genomic complexes. By way of non-limiting example, in some embodiments disruption may refer to changes in structural topology of one or more genomic complexes. In some embodiments, disruption, as used herein, may refer to changes in function of one or more genomic complexes without requiring impact or change to structural topology. For example, in some embodiments, methods may include disruption of structural topology of one or more genomic complexes. Without wishing to be bound by any theory, in some embodiments, disruption of genomic complexes may alter gene expression. Gene expression alteration may be or comprise upregulation of one or more genes relative to expression levels in absence of genomic complex disruption. Gene expression alteration may be or comprise downregulation of one or more genes relative to expression levels in absence of genomic complex disruption.

In some embodiments, disruption may be or comprise deleting one or more CTCF binding sites.

In some embodiments, disruption may be or comprise methylating one or more CTCF binding sites.

In some embodiments, disruption may be or comprise inducing degradation of non-coding RNA that is part of a genomic complex (e.g. between two CTCF binding sites/anchor sites).

In some embodiments, disruption may be or comprise interfering with assembly of one or more genomic complexes (e.g. a genomic complex that would otherwise form in absence of exogenous interference) by blocking resident non-coding RNA.

### Genetic Modification

In some embodiments, technologies (e.g. methods and/or compositions) provided by the present disclosure for altering a target gene may include site specific editing or mutating of a genomic sequence element (e.g., that participates in a genomic complex or transcription complex and/or is part of an gene associated therewith). For example, in some embodiments, an endogenous or naturally occurring anchor sequence may be altered to inactivate or delete an anchor sequence (e.g., thereby disrupting an anchor sequence-mediated conjunction or the genomic complex comprising said conjunction), or may be altered to mutate or replace an anchor sequence (e.g., to mutate or replace an anchor sequence with an altered anchor sequence that has an altered affinity, e.g., decreased affinity or increased affinity, to a nucleating protein) to modulate strength of a targeted conjunction. In some embodiments, for example, one or a plurality of exogenous anchor sequences can be incorporated into the genome of a subject to create a non-naturally occurring anchor sequence-mediated conjunction that incorporates a target gene, e.g., in order to silence a target gene. In some embodiments, an exogenous anchor sequence can form an anchor sequence-mediated conjunction with an endogenous anchor sequence. A nucleating protein may be, e.g., CTCF, cohesin, USF1, YY1, TAF3, ZNF143 binding motif, or another polypeptide that promotes formation of an anchor sequence-mediated conjunction.

In some embodiments, technologies as provided herein may include those that alter a target sequence (e.g. a sequence that is part of or participates in a targeted genomic complex).

In some embodiments, technologies as provided herein may include those that alter a target sequence (for example, an anchor sequence), which is a CTCF-binding motif:

(SEQ ID NO: 1)
N(T/C/G)N(G/A/T)CC(A/T/G)(C/G)(C/T/A)AG(G/A)(G/T)
GG(C/A/T)(G/A)(C/G)(C/T/A)(G/A/C),

where N is any nucleotide. A CTCF-binding motif may also be altered to be in the opposite orientation, e.g.,

| (SEQ ID NO: 2) | |
|---|---|
| | (G/A/C)(C/T/A)(C/G)(G/A)(C/A/T)GG(G/T)(G/A)GA |
| | (C/T/A)(C/G)(A/T/G)CC(G/A/T)N(T/C/G)N. |

An alteration can be introduced in a gene of a cell, e.g., in vitro, ex vivo, or in vivo.

In some cases, compositions and/or methods of the present disclosure are for altering chromatin structure, e.g., such that a two-dimensional representation of chromatin structure may change from that of a complex to a non-complex (or favor a non-complex over a complex) or vice versa, to alter a component of a genomic complex or transcription complex (e.g. a transcription factor and, e.g. its interaction with a genomic sequence), to inactivate a targeted CTCF-binding motif, e.g., an alteration abolishes CTCF binding thereby abolishing formation of a targeted conjunction, etc. In other examples, an alteration attenuates (e.g., decreases the level of) activity of a particular genomic complex component thereby decreasing or disrupting formation of a genomic complex (e.g., by altering a CTCF sequence to bind with less affinity to a nucleating protein). In some embodiments, a targeted alteration increases activity of a particular genomic complex component thereby increasing or maintaining formation of a genomic complex (e.g., by altering the CTCF sequence to bind with more affinity to a nucleating protein), thereby promoting formation of a targeted conjunction.

In some embodiments, provided modulating agents may comprise (i) a fusion molecule comprising an enzymatically inactive Cas polypeptide and a deaminating agent, or a nucleic acid encoding the fusion molecule; and (ii) a nucleic acid molecule (e.g. gRNA, PNA, BNA, etc), wherein the nucleic acid molecule targets a fusion molecule to a target sequence (e.g. in a genomic complex, e.g. in an anchor sequence-mediated conjunction) but not to at least one non-target anchor sequence (a “site-specific nucleic acid molecule”, such as described further herein).

In some embodiments, in order to introduce small mutations or a single-point mutation, a homologous recombination (HR) template can also be used. In some embodiments, an HR template is a single stranded DNA (ssDNA) oligo or a plasmid. In some embodiments, for example, for ssDNA oligo design, one may use around 100-150 bp total homology with a mutation introduced roughly in the middle, giving 50-75 bp homology arms.

In some embodiments, a nucleic acid molecule for targeting a target anchor sequence, e.g., a target sequence, is administered in combination with an HR template selected from:
(a) a nucleotide sequence comprising a target sequence of interest (e.g. target sequence that is part of or participates in a target genomic complex);
(b) a nucleotide sequence at least 75%, 80%, 85%, 90%, 95% identical to a target sequence of interest;
(c) a nucleotide sequence comprising a target sequence of interest having at least 1, 2, 3, 4, 5, but less than 15, 12 or 10 nucleotide additions, substitutions or deletions.

### Modifying Chromatin Structure

In some embodiments, methods provided herein modulate chromatin structure (e.g., genomic complexes, transcription complexes, or anchor sequence-mediated conjunctions) in order to modulate gene expression in a subject. Those skilled in the art reading the present specification will appreciate that modulations described herein may modulate chromatin structure in a way that would alter its two-dimensional representation (e.g., would add, alter, or delete a complex or a other anchor sequence-mediated conjunction); such modulations are referred to herein, in accordance with common parlance, as modulations or modification of a two-dimensional structure.

In some aspects, methods provided herein may comprise modifying a two-dimensional structure by altering a topology of a genomic complex or transcription complex, e.g., an anchor sequence-mediated conjunction, to modulate transcription of a nucleic acid sequence, wherein altered topology of a genomic complex or transcription complex, e.g., an anchor sequence-mediated conjunction, modulates transcription of a nucleic acid sequence.

In some aspects, methods provided herein may comprise modifying a two-dimensional structure chromatin structure by altering a topology of a plurality of genomic complexes or transcription complexes, e.g., anchor sequence-mediated conjunctions, to modulate transcription of a nucleic acid sequence, wherein altered topology modulates transcription of a nucleic acid sequence.

In some aspects, methods provided herein may comprise modulating transcription of a nucleic acid sequence by altering a genomic complex or transcription complex, e.g., an anchor sequence-mediated conjunction, that influences transcription of a nucleic acid sequence, wherein altering a genomic complex or transcription complex, e.g., an anchor sequence-mediated conjunction, modulates transcription of a nucleic acid sequence.

In some embodiments, altering a genomic complex or transcription complex, e.g., an anchor sequence-mediated conjunction, comprises modifying a chromatin structure, e.g., disrupting [reversible or irreversible] a topology of a genomic complex or transcription complex, e.g., an anchor sequence-mediated conjunction, altering one or more nucleotides in a genomic complex or transcription complex, e.g., an anchor sequence-mediated conjunction, [genetically modifying the sequence], epigenetically modifying [modulating DNA methylation at one or more sites] a genomic complex or transcription complex, e.g., an anchor sequence-mediated conjunction, or forming a non-naturally occurring anchor sequence-mediated conjunction. In some embodiments, altering a genomic complex or transcription complex, e.g., an anchor sequence-mediated conjunction, comprises modifying a chromatin structure. As appreciated by those of skill in the art, a given pair of anchor sequences may “breathe” in and out of an anchor sequence-mediated conjunction, though a given pair of anchor sequences may tend to be more or less often in a particular state (either in or out of a conjunction) depending on factors, such as, for example, cell type.

By “disruption” it is meant that formation and/or stability of a genomic complex or transcription complex, e.g., an anchor sequence-mediated conjunction, is negatively affected.

### Epigenetic Modification

In some embodiments, provided compositions and/or methods are described herein for altering a genomic complex or transcription complex by site specific epigenetic modification (e.g., methylation or demethylation).

In some embodiments, a modulating agent, e.g., fusion molecule, may cause epigenetic modification. For example, an endogenous or naturally occurring target sequence (e.g. a sequence within a target genomic complex or transcription complex) may be altered to increase its methylation (e.g., decreasing interaction of a component of a genomic complex or transcription complex (e.g. a transcription factor) with a portion of a genomic sequence, decreasing binding of a nucleating protein to the anchor sequence and disrupting or preventing an anchor sequence-mediated conjunction, or may be altered to decrease its methylation (e.g., interaction of a component of a genomic complex (e.g. a transcription factor) with a portion of a genomic sequence, increasing binding of a nucleating protein to an anchor sequence and promoting or increasing strength of an anchor sequence-mediated conjunction, etc.). In some particular embodiments, a modulating agent may be or comprise a fusion molecule, for example comprising a site-specific targeting moiety (such as any one of a targeting moieties as described herein) and an effector moiety, e.g., epigenetic modifying agent, wherein a site-specific targeting moiety targets a fusion molecule to a target anchor sequence but not to at least one non-target anchor sequence. In other embodiments, the targeting moiety targets the fusion molecule to a genomic sequence element associated with a target eRNA (or a genomic complex or transcription complex comprising the target eRNA). An epigenetic modifying agent can be any one of or any combination of epigenetic modifying agents as disclosed herein.

In some embodiments, for example, fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be guided to specific DNA sites by one or more RNA sequences (sgRNA) to modulate activity and/or expression of one or more target nucleic acids sequences (e.g., to methylate or demethylate a DNA sequence).

In some embodiments, fusion of a dCas9 with all or a portion of one or more effector domains of an epigenetic modifying agent (such as a DNA methylase or enzyme with a role in DNA demethylation) creates a chimeric protein that is useful in methods provided by the present disclosure. Accordingly, for example, in some embodiments, a nucleic acid encoding a dCas9-methylase fusion in combination with a site-specific gRNA or antisense DNA oligonucleotide that targets a fusion to a genomic complex component (such as a transcription factor, ncRNA (e.g., eRNA), CTCF binding motif, etc.), may together decrease affinity or ability of a component of a genomic complex or transcription complex to interact with a particular genomic sequence. In some embodiments, a nucleic acid encoding a dCas9-enzyme fusion in combination with a site-specific gRNA or antisense DNA oligonucleotide that targets a fusion to a genomic complex component (such as a transcription factor, ncRNA (e.g., eRNA), CTCF binding motif, etc.), may together increase affinity or ability of a component of a genomic complex or transcription complex to interact with a particular genomic sequence.

In some embodiments, all or a portion of one or more methylase, or enzyme with a role in DNA demethylation, effector domains are fused with an inactive nuclease, e.g., dCas9. In some embodiments, 1, 2, 3, 4, 5, 6, 7, 8, 9, 10, 11, 12, 13, 14, 15, 16, 17, 18, 19, 20, or more methylase, or enzyme with a role in DNA demethylation, effector domains (all or a biologically active portion) are fused with dCas9. Chimeric proteins as described herein may also comprise a linker, e.g., an amino acid linker. In some embodiments, a linker comprises 2 or more amino acids, e.g., one or more GS sequences. In some embodiment, fusion of Cas9 (e.g., dCas9) with two or more effector domains (e.g., of a DNA methylase or enzyme with a role in DNA demethylation) comprises one or more interspersed linkers (e.g., GS linkers) between domains. In some aspects, dCas9 is fused with 2-5 effector domains with interspersed linkers.

In embodiments, compositions and/or methods of the present disclosure may comprise a gRNA that specifically targets a sequence or component of a genomic complex or transcription complex (e.g. CTCF binding motif, ncRNA/eRNA, transcription factor, transcription regulator, etc.). In some embodiments, the sequence or component is associated with a particular type of gene or sequence, which may be associated with one or more diseases, disorders and/or conditions.

Epigenetic modifying agents useful in provided methods and/or compositions include agents that affect, e.g., DNA methylation, histone acetylation, and RNA-associated silencing. In some embodiments, methods provided herein may involve sequence-specific targeting of an epigenetic enzyme (e.g., an enzyme that generates or removes epigenetic marks, e.g., acetylation and/or methylation). In some embodiments, exemplary epigenetic enzymes that can be targeted to an anchor sequence using the CRISPR methods described herein include DNA methylases (e.g., DNMT3a, DNMT3b, DNMTL), enzymes with a role in DNA demethylation (e.g., the TET family enzymes catalyze oxidation of 5-methylcytosine to 5-hydroxymethylcytosine and higher oxidative derivatives), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012):1-18.

In some embodiments, an epigenetic modifying agent useful herein comprises a construct described in Koferle et al. Genome Medicine 7.59 (2015):1-3 (e.g., at Table 1), incorporated herein by reference.

Exemplary dCas9 fusion methods and compositions that are adaptable to methods and/or compositions of the present disclosure are known and are described, e.g., in Kearns et al., Functional annotation of native enhancers with a Cas9-histone demethylase fusion. Nature Methods 12, 401-403 (2015); and McDonald et al., Reprogrammable CRISPR/Cas9-based system for inducing site-specific DNA methylation. Biology Open 2016: doi: 10.1242/bio.019067.

In some embodiments, compositions and methods are described herein for reversibly disrupting a genomic complex or transcription complex, e.g., an anchor sequence-mediated conjunction. In some embodiments, for example, disruption may transiently modulate transcription, e.g., a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, compositions and/or methods provided herein may irreversibly disrupt a genomic complex or transcription complex, e.g., an anchor sequence-mediated conjunction.

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### EXAMPLES

### Example 1

The present Example describes a strategy to decrease expression of a gene (in this case, ARID1A) within a genomic complex as described herein.

This example describes technology for reducing level of a targeted genomic complex by using an agent that degrades and/or reduces half-life of a non-coding RNA (ncRNA) component of the genomic complex.

In this particular example, a targeting agent that is or comprises an oligonucleotide is designed and selected to hybridize specifically with the targeted ncRNA so that a hybridized duplex is generated, which hybridized duplex is susceptible to degradation by RNAse H. Typically, such a targeting agent is or comprises deoxyribonucleic acid.

In some embodiments, an agent as described in the present example is contacted with a system (e.g., with one or more cells) and is delivered to a site or location at which an active transcription complex comprising the ncRNA is present in the system. For example, in a system comprising one or more cells, the agent may be delivered to the nucleus; such delivery may be passive or active (e.g., may involve nuclear localization amino acid sequences associated with the agent).

In particular, this Example describes an agent comprising a deoxyribonucleic acid polymer that is complementary to a portion of a particular ncRNA that is transcribed from ARID1A promoter (5′-CTCTTCTCTCTTAAAATGGCTGCCTGTCTG-3′) (the “target ncRNA”) and is known to participate as a component of a genomic complex whose presence has been correlated with expression of the ARID1A gene. This complex has also been shown to contain YY1.

Deoxyribonucleic acid polymers in this Example may be chemically synthesized, e.g., by commercial vendors. Agents in this Example are reconstituted in sterile water.

In some embodiments, some or all of the residues in the oligonucleotide agents are linked together via phosphorothioate linkages. In some embodiments, all residues are linked via phosphorothioate linkages. In Table 2 below, the * refer to 2′-OMe (O-methyl) modifications of indicated sugar residues.

TABLE 2
Sequences of deoxyribonucleic acid polymer
targeting agents (e.g., oligonucleotide
agents) with or without nuclear localization
amino acid sequence PKKKRKV.

| SEQ ID NO | Sequence (5′-3′) |
|---|---|
| 9013 | rG*rA*rC*rA*rG*dGdCdAdGdCdCdAdTdTdTr |
| | U*rA*rA*rG*rA* |
| 9014 | Part 1: maleimide- |
| | rG*rA*rC*rA*rG*dGdCdAdGdCdCdAdTdTdTr |
| | U*rA*rA*rG*rA*; |
| | Part II: PKKKRKV-C |

HEK293T cells are transfected or electroporated with the agents described in Table 2 or with otherwise comparable agents whose nucleotide sequence is scrambled relative to that presented in Table 2 and/or is otherwise not sufficiently complementary to specifically hybridize with any particular RNA sequence in the human genome under conditions described in this example.

At 72 hr post-transfection, cells are harvested for RNA extraction and cDNA synthesis using commercially available reagents and protocols (Qiagen; Thermo Fisher Scientific), and genomic DNA is extracted (Qiagen). Resulting cDNA is used for quantitative real-time PCR (Thermo Fisher Scientific). Genomic locus-specific quantitative PCR probes/primers are multiplexed with internal control quantitative PCR probes/primers, and gene expression is subsequently analyzed using a real-time PCR kit (Applied Biosystems, Thermo Fisher Scientific). ARID1A-specific quantitative PCR probes/primers (Assay ID Hs00153408_m1, Thermo Fisher Scientific) are multiplexed with internal control quantitative PCR probes/primers, which are either PPIB (Assay ID Hs00168719_m1, Thermo Fisher Scientific) or GAPDH (Assay ID Hs02786624_g1, Thermo Fisher Scientific) using FAM-MGB or VIC-MGB dyes, respectively. Gene expression is subsequently analyzed using a real time PCR kit (Applied Biosystems, Thermo Fisher Scientific).

Additionally, probes specific for the ncRNA transcribed from the ARID1A promoter (5′-CTCTTCTCTCTTAAAATGGCTGCCTGTCTG-3′) are multiplexed with internal control quantitative PCR probes/primers, which are either PPIB (Assay ID Hs00168719_m1, Thermo Fisher Scientific) or GAPDH (Assay ID Hs02786624_g1, Thermo Fisher Scientific) using FAM-MGB or VIC-MGB dyes, respectively. Gene expression is subsequently analyzed using a real time PCR kit (Applied Biosystems, Thermo Fisher Scientific).

Cells transfected with one or more agents described in Table 2 are expected to show reduced ARID1A expression and reduced ncRNA transcribed from the ARID1A promoter.

To determine the extent to which agents described in Table 2 confer changes to assembly and/or stability of a genomic complex that assembles in proximity to ARID1A promoter, chromatin immunoprecipitations are performed using antibodies against a protein known to be enriched in the genomic complex, such as cohesin, RNA polymerase, or components of Mediator complex, as described in a previous Example. A quantitative PCR assay is then performed as described in a previous Example. Primer sequences used for amplification reactions are as follows: 5′-GGGAATGAGCCGGGAGAG′-3′, 5′-TGCGCTGCGCTCGCTCCT-3′.

Diminished input-normalized amplification, by about 5% to about 100%, indicates reduced assembly and/or stability (e.g., half-life) of the targeted genomic complex.

To determine the extent to which agents described in Table 2 confer changes to proximity of the ARID1A promoter to enhancers that regulate ARID1A expression, a 4C-seq assay is performed as described in Example 1. Primer sequences used for amplification reactions with a long template PCR reaction (Roche) are as follows: ND263926_f 5′-TGGACTGAATCGTTGACATG-3′ and ND263926_r 5′-CTTTCCCGTTGCCACTGC-3′.

A diminished number of sequencing reads compared to non-targeting control, by about 5% to about 100%, indicates reduced assembly and/or stability (e.g., half-life) of the targeted genomic complex, suggesting that one or more agents described in Table 2 are sufficient to disrupt association of the ARID1A promoter with one or more enhancers that regulate ARID1A expression. A 4C-seq assay with the ARID1A promoter as bait/viewpoint in a non-targeting control identifies enhancers that interact with the ARID1A promoter. If, in a 4C-seq assay, an agent targeted to ARID1A promoter reduces the probability that a given enhancer is observed in proximity to the ARID1A promoter, we will determine if targeting one or more agents described in Table 2 to that enhancer region leads to the same effect as does targeting the same agent(s) to the ARID1A promoter.

### Example 2

The present Example describes a strategy to decrease expression of a gene (in this case, ARID1A) within a genomic complex as described herein. This example describes technology for reducing level of a targeted genomic complex by using an agent that degrades and/or reduces half-life of a non-coding RNA (ncRNA) component of the genomic complex.

In this particular Example, a targeting agent that is or comprises a ribonucleic acid duplex polymer is designed and selected to hybridize specifically with the targeted ncRNA so that an RNA/ncRNA duplex is generated, which RNA/ncRNA duplex is susceptible to siRNA-mediated degradation, thereby interfering with the targeted ncRNA's participation in the genomic complex. In particular, this Example describes an agent comprising a ribonucleic acid duplex polymer with one strand complementary to a portion of a particular ncRNA that is transcribed from the ARID1A promoter (5′-CTCTTCTCTCTTAAAATGGCTGCCTGTCTG-3′) (the “target ncRNA”) and is known to participate as a component of a genomic complex whose presence has been correlated with expression of the ARID1A gene. This complex has also been shown to contain YY1.

Ribonucleic acid polymers in this Example may be chemically synthesized, e.g., by commercial vendors. Agents in this Example are reconstituted in sterile water.

TABLE 3
Sequences of ribonucleic acid duplex polymer
targeting agents (e.g., oligonucleotide
agents) with or without nuclear localization
amino acid sequence PKKKRKV.

| SEQ ID NO | Sequence (5′-3′) |
|---|---|
| 9029 | antisense strand: rUrUrGrArCrArGrGrCrAr |
| | GrCrCrArUrUrUrUrArArGrArG; |
| | Sense Strand :rCrUrCrUrUrArArArArUrGrGr |
| | CrUrGrCrCrUrGrUrC |
| 9030 | antisense strand: rUrUrGrArCrArGrGrCrAr |
| | GrCrCrArUrUrUrUrArArGrArG; |
| | Sense Strand Two Parts |
| | Part I: rCrUrCrUrUrArArArArUrGrGrCrUrGr |
| | CrCrUrGrUrC-maleimide; |
| | Part II: PKKKRKV-C |

HEK293T cells are transfected or electroporated with the agents described in Table 3 or with otherwise comparable agents whose nucleotide sequence is scrambled relative to that presented in Table 3 and/or is otherwise not sufficiently complementary to specifically hybridize with any particular RNA sequence in the human genome under conditions described in this example.

At 72 hr post-transfection, cells are harvested for RNA extraction and cDNA synthesis using commercially available reagents and protocols (Qiagen; Thermo Fisher Scientific), and genomic DNA is extracted (Qiagen). Resulting cDNA is used for quantitative real-time PCR (Thermo Fisher Scientific). Genomic locus-specific quantitative PCR probes/primers are multiplexed with internal control quantitative PCR probes/primers and gene expression is subsequently analyzed using a real-time PCR kit (Applied Biosystems, Thermo Fisher Scientific). ARID1A-specific quantitative PCR probes/primers (Assay ID Hs00153408_m1, Thermo Fisher Scientific) are multiplexed with internal control quantitative PCR probes/primers, which are either PPIB (Assay ID Hs00168719_m1, Thermo Fisher Scientific) or GAPDH (Assay ID Hs02786624_g1, Thermo Fisher Scientific) using the FAM-MGB and VIC-MGB dyes, respectively. Gene expression is subsequently analyzed using a real time PCR kit (Applied Biosystems, Thermo Fisher Scientific).

Additionally, probes specific for the ncRNA transcribed from ARID1A promoter (5′-CTCTTCTCTCTTAAAATGGCTGCCTGTCTG-3′) are multiplexed with internal control quantitative PCR probes/primers, which are either PPIB (Assay ID Hs00168719_m1, Thermo Fisher Scientific) or GAPDH (Assay ID Hs02786624_g1, Thermo Fisher Scientific) using FAM-MGB or VIC-MGB dyes, respectively. Gene expression is subsequently analyzed using a real time PCR kit (Applied Biosystems, Thermo Fisher Scientific).

Cells transfected with one or more agents described in Table 3 are expected to show reduced ARID1A expression and reduced ncRNA transcribed from ARID1A promoter.

To determine the extent to which agents described in Table 3 confer changes to assembly and/or stability of a genomic complex that assembles in proximity to ARID1A promoter, chromatin immunoprecipitations are performed using antibodies against a protein known to be enriched in the genomic complex, such as cohesin, RNA polymerase, or components of Mediator complex, as described in a previous Example. A quantitative PCR assay is then performed as described in a previous Example. Primer sequences used for amplification reactions are as follows: 5′-GGGAATGAGCCGGGAGAG′-3′, 5′-TGCGCTGCGCTCGCTCCT-3′.

Diminished input-normalized amplification, by about 5% to about 100%, indicates reduced assembly and/or stability (e.g., half-life) of the targeted genomic complex.

To determine the extent to which agents described in Table 3 confer changes to proximity of the ARID1A promoter to enhancers that regulate ARID1A expression, a 4C-seq assay is performed as described in Example 1. Primer sequences used for amplification reactions with a long template PCR reaction (Roche) are as follows: ND263926_f 5′-TGGACTGAATCGTTGACATG-3′ and ND263926_r 5′-CTTTCCCGTTGCCACTGC-3′.

A diminished number of sequencing reads compared to non-targeting control, by about 5% to about 100%, indicates reduced assembly and/or stability (e.g., half-life) of the targeted genomic complex, suggesting that one or more agents described in Table 3 are sufficient to disrupt association of the ARID1A promoter with one or more enhancers that regulate ARID1A expression. A 4C-seq assay with the ARID1A promoter as bait/viewpoint in a non-targeting control identifies enhancers that interact with the ARID1A promoter.

If, in a 4C-seq assay, an agent targeted to ARID1A promoter reduces the probability that a given enhancer is observed in proximity to the ARID1A promoter, we will determine if targeting one or more agents described in Table 3 to that enhancer region leads to the same effects as does targeting the same agent(s) to the ARID1A promoter.

### Example 3

The present Example describes a strategy to decrease expression of a gene (in this case, ARID1A) within a genomic complex as described herein. This example describes technology for reducing level of a targeted genomic complex by using an agent that directly binds a non-coding RNA (ncRNA) component of the genomic complex and thus sterically blocks the targeted ncRNA from interacting with other constituents of the genomic complex.

In this particular example, a targeting agent that is or comprises an oligonucleotide is designed and selected to hybridize specifically with the targeted ncRNA so that a hybridized duplex is generated. In this Example, the agent is or comprises a single stranded ribonucleic acid polymer that is complementary to the ncRNA.

In some embodiments, an agent as described in the present example is contacted with a system (e.g., with one or more cells) and is delivered to a site or location at which an active transcription complex comprising the ncRNA is present in the system. For example, in a system comprising one or more cells, the agent may be delivered to the nucleus; such delivery may be passive or active (e.g., may involve nuclear localization amino acid sequences associated with the agent).

In particular, this Example describes an agent comprising a single-stranded ribonucleic acid polymer that is complementary to a portion of a particular ncRNA that is transcribed from the ARID1A promoter (5′-CTCTTCTCTCTTAAAATGGCTGCCTGTCTG-3′) (the “target ncRNA”) and is known to participate as a component of a genomic complex whose presence has been correlated with expression of the ARID1A gene. This complex has also been shown to contain YY1.

Ribonucleic acid polymers in this Example may be chemically synthesized, e.g., by commercial vendors. Agents in this Example are reconstituted in sterile water.

TABLE 4
Sequences of ribonucleic acid polymer
targeting agents (e.g., oligonucleotide
agents) with or without nuclear
localization amino acid sequence PKKKRKV.

| SEQ ID NO | Sequence (5′-3′) |
|---|---|
| 9031 | rCrArGrArCrArGrGrCrArGrCrCrArUrUrUrUrAr |
| | ArGrArGrArGrArArGrArG |
| 9032 | Part 1: maleimide- |
| | rCrArGrArCrArGrGrCrArGrCrCrArUrUrUrUrAr |
| | ArGrArGrArGrArArGrArG; |
| | Part II: PKKKRKV-C |

HEK293T cells are transfected or electroporated with the agents described in Table 4 or with otherwise comparable agents whose nucleotide sequence is scrambled relative to that presented in Table 4 and/or is otherwise not sufficiently complementary to specifically hybridize with any particular RNA sequence in the human genome under conditions described in this example.

At 72 hr post-transfection, cells are harvested for RNA extraction and cDNA synthesis using commercially available reagents and protocols (Qiagen; Thermo Fisher Scientific), and genomic DNA is extracted (Qiagen). Resulting cDNA is used for quantitative real-time PCR (Thermo Fisher Scientific). Genomic locus-specific quantitative PCR probes/primers are multiplexed with internal control quantitative PCR probes/primers, and gene expression is subsequently analyzed using a real-time PCR kit (Applied Biosystems, Thermo Fisher Scientific). ARID1A-specific quantitative PCR probes/primers (Assay ID Hs00153408_m1, Thermo Fisher Scientific) are multiplexed with internal control quantitative PCR probes/primers, which are either PPIB (Assay ID Hs00168719_m1, Thermo Fisher Scientific) or GAPDH (Assay ID Hs02786624_g1, Thermo Fisher Scientific) using FAM-MGB or VIC-MGB dyes, respectively. Gene expression is subsequently analyzed using a real time PCR kit (Applied Biosystems, Thermo Fisher Scientific). Additionally, probes specific for the ncRNA transcribed from ARID1A promoter (5′-CTCTTCTCTCTTAAAATGGCTGCCTGTCTG-3′) are multiplexed with internal control quantitative PCR probes/primers, which are either PPIB (Assay ID Hs00168719_m1, Thermo Fisher Scientific) or GAPDH (Assay ID Hs02786624_g1, Thermo Fisher Scientific) using FAM-MGB or VIC-MGB dyes, respectively. Gene expression is subsequently analyzed using a real time PCR kit (Applied Biosystems, Thermo Fisher Scientific).

Cells transfected with one or more agents described in Table 4 are expected to show reduced ARID1A expression and reduced ncRNA transcribed from ARID1A promoter.

To determine the extent to which agents described in Table 4 confer changes to assembly and/or stability of a genomic complex that assembles in proximity to ARID1A promoter, chromatin immunoprecipitations are performed using antibodies against a protein known to be enriched in the genomic complex, such as cohesin, RNA polymerase, or components of Mediator complex—followed by quantitative PCR assay are performed as described in a previous Example. Primer sequences used for amplification reactions are as follows: 5′-GGGAATGAGCCGGGAGAG′-3′, 5′-TGCGCTGCGCTCGCTCCT-3′.

Diminished input-normalized amplification, by about 5% to about 100%, indicates reduced assembly and/or stability (e.g., half-life) of the targeted genomic complex.

To determine the extent to which agents described in Table 4 confer changes to proximity of the ARID1A promoter to enhancers that regulate ARID1A expression, a 4C-seq assay is performed as described in Example 1. Primer sequences used for amplification reactions with a long template PCR reaction (Roche) are as follows: ND263926_f 5′-TGGACTGAATCGTTGACATG-3′ and ND263926_r 5′-CTTTCCCGTTGCCACTGC-3′.

A diminished number of sequencing reads compared to non-targeting control, by about 5% to about 100%, indicates reduced assembly and/or stability (e.g., half-life) of the targeted genomic complex, suggesting that the one or more agents described in Table 4 are sufficient to disrupt association of the ARID1A promoter with one or more enhancers that regulate ARID1A expression.

### Example 4

The present Example describes a strategy to decrease expression of a gene (in this case, ARID1A) within a genomic complex as described herein. This example describes technology for reducing level of a targeted genomic complex by using an agent that directly binds a non-coding RNA (ncRNA) component of the genomic complex and thus sterically blocks the targeted ncRNA from interacting with other constituents of genomic complex. In this particular Example, the agent is a PNA (peptide nucleic acid) with ribonucleic acid bases, the sequence of which are complementary to at least a portion of the ncRNA.

In some embodiments, an agent as described in the present example is contacted with a system (e.g., with one or more cells) and is delivered to a site or location at which an active transcription complex comprising the ncRNA is present in the system. For example, in a system comprising one or more cells, the agent may be delivered to the nucleus; such delivery may be passive or active (e.g., may involve nuclear localization amino acid sequences associated with the agent).

In particular, this Example describes an agent comprising a PNA whose ribonucleic acid sequence is complementary to a portion of a particular ncRNA that is transcribed from the ARID1A promoter (5′-CTCTTCTCTCTTAAAATGGCTGCCTGTCTG-3′) (the “target ncRNA”) and is known to participate as a component of a genomic complex whose presence has been correlated with expression of the ARID1A gene. This complex has also been shown to contain YY1.

PNAs in this example may be chemically synthesized, e.g., by commercial vendors. Agents in this Example are reconstituted in sterile water.

TABLE 5
Sequences of peptide ribonucleic acid polymer
targeting agents with or without nuclear
localization amino acid sequence PKKKRKV.

| SEQ ID | |
|---|---|
| NO | Sequence (5′-3′) |
| 9060 | PNA: PKKKRKV- |
| | rCrArGrArCrArGrGrCrArGrCrCrArUrUrUrUrArAr |
| | GrArGrArGrArArGrArG |
| 9061 | PNA: PKKKRKV- |
| | rCrArG*rArCrA*rGrGrC*rArGrC*rCrArU*rUrUrU |
| | *rArArG*rArGrA*rGrArA*rGrArG* |

HEK293T cells are transfected or electroporated with the agents described in Table 5 or with otherwise comparable agents whose nucleotide sequence is scrambled relative to that presented in Table 5 and/or is otherwise not sufficiently complementary to specifically hybridize with any particular RNA sequence in the human genome under conditions described in this example.

At 72 hr post-transfection, cells are harvested for RNA extraction and cDNA synthesis using commercially available reagents and protocols (Qiagen; Thermo Fisher Scientific), and genomic DNA is extracted (Qiagen). Resulting cDNA is used for quantitative real-time PCR (Thermo Fisher Scientific). Genomic locus-specific quantitative PCR probes/primers are multiplexed with internal control quantitative PCR probes/primers and gene expression is subsequently analyzed using a real-time PCR kit (Applied Biosystems, Thermo Fisher Scientific). ARID1A-specific quantitative PCR probes/primers (Assay ID Hs00153408_m1, Thermo Fisher Scientific) are multiplexed with internal control quantitative PCR probes/primers, which are either PPIB (Assay ID Hs00168719_m1, Thermo Fisher Scientific) or GAPDH (Assay ID Hs02786624_g1, Thermo Fisher Scientific) using FAM-MGB or VIC-MGB dyes, respectively. Gene expression is subsequently analyzed using a real time PCR kit (Applied Biosystems, Thermo Fisher Scientific).

Additionally, probes specific for the ncRNA transcribed from ARID1A promoter (5′-CTCTTCTCTCTTAAAATGGCTGCCTGTCTG-3′) are multiplexed with internal control quantitative PCR probes/primers, which are either PPIB (Assay ID Hs00168719_m1, Thermo Fisher Scientific) or GAPDH (Assay ID Hs02786624_g1, Thermo Fisher Scientific) using FAM-MGB or VIC-MGB dyes, respectively. Gene expression is subsequently analyzed using a real time PCR kit (Applied Biosystems, Thermo Fisher Scientific).

Cells transfected with one or more agents described in Table 5 are expected to show reduced ARID1A expression and reduced ncRNA transcribed from ARID1A promoter.

To determine the extent to which agents described in Table 5 confer changes to assembly and/or stability of a genomic complex that assembles in proximity to ARID1A promoter, chromatin immunoprecipitations are performed using antibodies against a protein known to be enriched in the genomic complex, such as cohesin, RNA polymerase, or components of Mediator complex, as described in a previous Example. A quantitative PCR assay is then performed as described in a previous Example. Primer sequences used for amplification reactions are as follows: 5′-GGGAATGAGCCGGGAGAG′-3′, 5′-TGCGCTGCGCTCGCTCCT-3′.

Diminished input-normalized amplification, by about 5% to about 100%, indicates reduced assembly and/or stability (e.g., half-life) of the targeted genomic complex.

To determine the extent to which agents described in Table 5 confer changes to proximity of the ARID1A promoter to enhancers that regulate ARID1A expression, a 4C-seq assay is performed as described in Example 1. Primer sequences used for amplification reactions with a long template PCR reaction (Roche) are as follows: ND263926_f 5′-TGGACTGAATCGTTGACATG-3′ and ND263926_r 5′-CTTTCCCGTTGCCACTGC-3′.

A diminished number of sequencing reads compared to non-targeting control, by about 5% to about 100%, indicates reduced assembly and/or stability (e.g., half-life) of the targeted genomic complex, suggesting that one or more agents described in Table 5 are sufficient to disrupt association of ARID1A promoter with one or more enhancers that regulate ARID1A expression.

### Example 5

The present Example describes a strategy to decrease expression of a gene (in this case, ARID1A) within a genomic complex as described herein. This example describes technology for reducing level of a targeted genomic complex by using an agent that directly binds a non-coding RNA (ncRNA) component of the genomic complex and thus sterically blocks the targeted ncRNA from interacting with other constituents of the genomic complex.

In this particular example, a targeting agent that is or comprises an oligonucleotide is designed and selected to hybridize specifically with the targeted ncRNA so that a hybridized duplex is generated. In this Example, the agent is or comprises a single stranded ribonucleic acid polymer that is complementary to the targeted ncRNA and is covalently attached to the 3′ end of the tracr RNA. The agent is targeted to a specific genomic complex via targeted guide RNAs complexed with dCas9.

In particular, this Example describes an agent comprising a ribonucleic acid polymer that is complementary to a portion of a particular ncRNA that is transcribed from the ARID1A promoter (5′-CTCTTCTCTCTTAAAATGGCTGCCTGTCTG-3′) (the “target ncRNA”) and is known to participate as a component of a genomic complex whose presence has been correlated with expression of the ARID1A gene. This complex has also been shown to contain YY1.

Ribonucleic acid polymers in this Example may be chemically synthesized, e.g., by commercial vendors. Agents in this Example are reconstituted in sterile water.

TABLE 6
Sequences of ribonucleic acid polymer
targeting agents (e.g., oligonucleotide
agents) comprising tracr RNA covalently
linked to a ribonucleic acid sequence
that is complementary to targeted ncRNA.

| SEQ | |
|---|---|
| ID NO | Sequence (5′-3′) |
| 9062 | rGrUrUrGrGrArArCrCrArUrUrCrArArArArCrArGrC |
| | rArUrArGrCrArArGrUrUrArArArArUrArArGrGrCrU |
| | rArGrUrCrCrGrUrUrArUrCrArArCrUrUrGrArArArA |
| | rArGrUrGrGrCrArCrCrGrArGrUrCrGrGrUrGrCrUrU |
| | rUrUrUrCrUrCrUrUrCrUrCrUrCrUrUrArArArArUrG |
| | rGrCrUrGrCrCrUrGrUrCrUrG |

TABLE 7
Sequences of ribonucleic acid polymer
targeting agents (e.g., oligonucleotide
agents) comprising guide RNAs that target
dCas9/guideRNA/tracr RNA complexes to the
ARIDIA promoter region.

| SEQ | |
|---|---|
| ID NO | Sequence (5′-3′) |
| 9064 | rGrGrGrUrGrCrGrGrGrGrCrArGrArGrUrGrUrGrCrG |
| | rUrUrUrUrArGrArGrCrUrArUrGrCrUrGrUrUrUrUrG |
| 9065 | rGrGrUrGrCrGrGrGrGrCrArGrArGrUrGrUrGrCrGrG |
| | rUrUrUrUrArGrArGrCrUrArUrGrCrUrGrUrUrUrUrG |
| 9066 | rCrGrGrGrGrCrArGrArGrUrGrUrGrCrGrGrGrGrArG |
| | rUrUrUrUrArGrArGrCrUrArUrGrCrUrGrUrUrUrUrG |
| 9067 | rGrGrGrCrGrCrUrGrGrGrGrGrCrUrCrArGrGrUrUrG |
| | rUrUrUrUrArGrArGrCrUrArUrGrCrUrGrUrUrUrUrG |
| 9068 | rArArGrGrGrCrCrUrGrGrCrCrGrGrGrGrGrUrGrArG |
| | rUrUrUrUrArGrArGrCrUrArUrGrCrUrGrUrUrUrUrG |
| 9069 | rUrGrArGrGrGrGrGrUrGrArGrGrGrGrGrCrGrCrUrG |
| | rUrUrUrUrArGrArGrCrUrArUrGrCrUrGrUrUrUrUrG |
| 9070 | rGrGrGrGrCrArGrArGrUrGrUrGrCrGrGrGrGrArArG |
| | rUrUrUrUrArGrArGrCrUrArUrGrCrUrGrUrUrUrUrG |
| 9071 | rGrCrGrGrGrGrArArGrGrGrCrCrUrGrGrCrCrGrGrG |
| | rUrUrUrUrArGrArGrCrUrArUrGrCrUrGrUrUrUrUrG |
| 9072 | rUrCrArCrCrCrCrCrUrCrArCrCrCrCrCrGrGrCrCrG |
| | rUrUrUrUrArGrArGrCrUrArUrGrCrUrGrUrUrUrUrG |
| 9073 | rCrArArGrArCrCrCrCrUrGrGrArGrUrUrCrGrArArG |
| | rUrUrUrUrArGrArGrCrUrArUrGrCrUrGrUrUrUrUrG |

HEK293T cells are transfected or electroporated with the agents described in Table 6 or 7 or with otherwise comparable agents whose nucleotide sequence is scrambled relative to that presented in Table 6 or 7 and/or is otherwise not sufficiently complementary to specifically hybridize with any particular RNA sequence in the human genome under conditions described in this example.

At 72 hr post-transfection, cells are harvested for RNA extraction and cDNA synthesis using commercially available reagents and protocols (Qiagen; Thermo Fisher Scientific), and genomic DNA is extracted (Qiagen). Resulting cDNA is used for quantitative real-time PCR (Thermo Fisher Scientific). Genomic locus-specific quantitative PCR probes/primers are multiplexed with internal control quantitative PCR probes/primers and gene expression is subsequently analyzed using a real-time PCR kit (Applied Biosystems, Thermo Fisher Scientific). ARID1A-specific quantitative PCR probes/primers (Assay ID Hs00153408_m1, Thermo Fisher Scientific) are multiplexed with internal control quantitative PCR probes/primers, which are either PPIB (Assay ID Hs00168719_m1, Thermo Fisher Scientific) or GAPDH (Assay ID Hs02786624_g1, Thermo Fisher Scientific) using FAM-MGB or VIC-MGB dyes, respectively. Gene expression is subsequently analyzed using a real time PCR kit (Applied Biosystems, Thermo Fisher Scientific). Additionally, probes specific for the ncRNA transcribed from ARID1A promoter (5′-CTCTTCTCTCTTAAAATGGCTGCCTGTCTG-3′) are multiplexed with internal control quantitative PCR probes/primers, which are either PPIB (Assay ID Hs00168719_m1, Thermo Fisher Scientific) or GAPDH (Assay ID Hs02786624_g1, Thermo Fisher Scientific) using FAM-MGB or VIC-MGB dyes, respectively. Gene expression is subsequently analyzed by a real time PCR kit (Applied Biosystems, Thermo Fisher Scientific).

Cells transfected with one or more agents in described Table 6 or 7 are expected to show reduced ARID1A expression and reduced ncRNA transcribed from ARID1A promoter.

To determine the extent to which agents described in Tables 6 and 7 confer changes to assembly of a genomic complex that assembles in proximity to ARID1A promoter, chromatin immunoprecipitations are performed using antibodies a protein known to be enriched in the genomic complex, such as cohesin, RNA polymerase, or components of Mediator complex, as described in a previous Example. A quantitative PCR assay is then performed as described in a previous Example. Primer sequences used for amplification reactions are as follows: 5′-GGGAATGAGCCGGGAGAG′-3′, 5′-TGCGCTGCGCTCGCTCCT-3′.

Diminished input-normalized amplification, by about 5% to about 100%, indicates reduced assembly and/or stability (e.g., half-life) of the targeted genomic complex.

To determine the extent to which agents described in Table 6 and 7 confer changes to proximity of the ARID1A promoter to enhancers that regulate ARID1A expression, a 4C-seq assay is performed as described in Example 1. Primer sequences used for amplifications reaction with a long template PCR reaction (Roche) are as follows: ND263926_f 5′-TGGACTGAATCGTTGACATG-3′ and ND263926_r 5′-CTTTCCCGTTGCCACTGC-3′.

A diminished number of sequencing reads compared to a non-targeting control, by about 5% to about 100%, indicates reduced assembly and/or stability (e.g., half-life) of the targeted genomic complex, suggesting that one or more agents described in Tables 6 and 7 are sufficient to disrupt association of the ARID1A promoter with one or more enhancers that regulate ARID1A expression. A 4C-seq assay with the ARID1A promoter as bait/viewpoint in a non-targeting control identifies enhancers that interact with the ARID1A promoter. If, in a 4C-seq assay, an agent targeted to ARID1A promoter reduces the probability that a given enhancer is observed in proximity to the ARID1A promoter, we will determine if targeting one or more agents described in Table 6 and 7 to that enhancer region leads to the same effects as does targeting the same agent(s) to the ARID1A promoter.

### EQUIVALENTS

It is to be understood that while the invention has been described in conjunction with the detailed description thereof, the foregoing description is intended to illustrate and not limit the scope of the invention, which is defined by the scope of the appended claims. Some aspects, advantages, and modifications are within the scope of the following claims.

We claim:

1. A fusion molecule, comprising:
a targeting moiety that binds to an enhancer RNA (eRNA), wherein the targeting moiety comprises a nucleic acid with a length of 10-50 nucleotides, and
an effector moiety covalently linked to the targeting moiety that modulates, e.g., increases or decreases, expression of a gene regulated by the eRNA.

2. The fusion molecule of claim 1, wherein the targeting moiety comprises no more than 50, 40, 30, or 20 nucleotides.

3. The fusion molecule of claim 1 or 2, wherein the fusion molecule comprises no more than 1300, 1200, 1100, 1000, 900, 800, 700, 600, 500, 400, 300, 200, or 100 amino acids.

4. The fusion molecule of any of claims 1-3, wherein the effector moiety does not comprise a CRISPR protein, e.g., does not comprise Cas9.

5. The fusion molecule of any of claims 1-4, wherein the effector moiety does not comprise CTCF or a functional fragment thereof.

6. The fusion molecule of any of claims 1-5, wherein the effector moiety does not bind to CTCF.

7. The fusion molecule of any of claims 1-6, wherein the effector moiety comprises an enzyme.

8. The fusion molecule of any of claims 1-7, wherein the effector moiety is capable of recruiting an endogenous protein, e.g., a protein endogenous to a cell, to the targeting moiety and/or eRNA.

9. The fusion molecule of any of claims 1-8, wherein the effector moiety comprises a genetic modifying moiety, e.g., chosen from a clustered regulatory interspaced short palindromic repeat (CRISPR) system, zinc finger nucleases (ZFNs), and Transcription Activator-Like Effector-based Nucleases (TALEN).

10. The fusion molecule of any of claims 1-9, wherein the effector moiety comprises an epigenetic modifying moiety, e.g., chosen from a DNA methylase (e.g., DNMT3a, DNMT3b, DNMTL); a DNA demethylation enzyme (e.g., members of the TET family); a histone methyltransferase; a histone deacetylase (e.g., HDAC1, HDAC2, HDAC3); sirtuin 1, 2, 3, 4, 5, 6, or 7; lysine-specific histone demethylase 1 (LSD1); histone-lysine-N-methyltransferase (Setdb1); euchromatic histone-lysine N-methyltransferase 2 (G9a); histone-lysine N-methyltransferase (SUV39H1); enhancer of zeste homolog 2 (EZH2); viral lysine methyltransferase (vSET); histone methyltransferase (SET2); or protein-lysine N-methyltransferase (SMYD2).

11. The fusion molecule of any of claims 1-10, wherein the effector moiety comprises a cleavable moiety, e.g., a moiety linked to the targeting moiety via a thrombin cleavable CPRSC linker.

12. The fusion molecule of any of claims 1-11, wherein the effector moiety comprises a small molecule.

13. The fusion molecule of any of claims 1-12, wherein the targeting moiety further comprises a protein or small molecule, e.g., an RNA-binding protein.

14. The fusion molecule of any of claims 1-12, wherein the targeting moiety consists essentially of the nucleic acid with a length of 10-50 nucleotides.

15. The fusion molecule of any of claims 1-14, wherein the nucleic acid comprises one or more of deoxyribonucleic acids; ribonucleic acids, nucleic acid analogs (e.g., one or more of a peptide nucleic acid (PNA), a peptide-oligonucleotide conjugate, a locked nucleic acid (LNA), a bridged nucleic acid (BNA)); linkers; and combinations thereof.

16. The fusion molecule of any of claims 1-15, wherein the nucleic acid comprises one or more phosphorothioate bonds, e.g., between a pair of nucleic acids or nucleic acid analogs.

17. The fusion molecule of any of claims 1-16, wherein the nucleic acid comprises a sequence with at least 80, 85, 90, 95, 99, or 100% identity to SEQ ID NOs: 9013-9073, or having no more than 10, 9, 8, 7, 6, 5, 4, 3, 2, or 1 alterations (e.g., none) relative to a sequence selected from SEQ ID NOs: 9013-9073.

18. A cell comprising the fusion molecule of any preceding claim.

19. A reaction mixture comprising the fusion molecule of any of claims 1-17 and a cell.

20. A method of treating a patient having aberrant expression of a target gene, the method comprising:
administering to the patient the fusion molecule of any of claims 1-17,
thereby treating the patient having aberrant expression of a target gene.

21. A method of decreasing expression of a target gene in a cell, the method comprising:
contacting the cell with the fusion molecule of any of claims 1-17,
thereby decreasing expression of the target gene.

22. The method or cell of any of claims 18, 20, or 21, wherein the cell comprises a genomic complex or transcription complex comprising the eRNA.

23. The method or cell of claim 22, wherein the level of genomic complex or transcription complex comprising the eRNA is altered (e.g., decreased) when the fusion molecule is present relative to when it is absent.

24. The method or cell of claim 22, wherein the stability of the genomic complex or transcription complex at a target site is altered (e.g., decreased) when the fusion molecule is present relative to when it is absent.

25. The method or cell of any of claims 22-24, wherein the cell comprises a genomic complex component or transcription factor that binds the eRNA.

26. The method or cell of claim 25, wherein the level of genomic complex component or transcription factor binding the eRNA is altered (e.g., decreased) when the fusion molecule is present relative to when it is absent.

27. The method or cell of claim 25, wherein the stability of the transcription factor or genomic complex component at a target site is altered (e.g., decreased) when the fusion molecule is present relative to when it is absent.

28. The method or cell of either of claim 24 or 27, wherein the target site is associated with expression of the target gene, e.g., and is selected from a promoter, enhancer, transcription start site, or coding sequence associated with the target gene.

29. A method of modulating (e.g., inhibiting) a genomic complex or transcription complex, the method comprising:
contacting the genomic complex or transcription complex with the fusion molecule of any of claims 1-17,
wherein:
(a) the level of genomic complex or transcription complex comprising the eRNA is altered (e.g., decreased) when the fusion molecule is present relative to when it is absent,
(b) the transcription complex comprises a transcription factor that binds the eRNA and the level of transcription complex comprising the transcription factor is altered (e.g., decreased) when the fusion molecule is present relative to when it is absent,
(c) the genomic complex comprises a genomic complex component that binds the eRNA and the level of genomic complex comprising the genomic complex component is altered (e.g., decreased) when the fusion molecule is present relative to when it is absent,
(d) the occupancy of the genomic complex or transcription complex at a target site is altered (e.g., decreased) when the fusion molecule is present relative to when it is absent, or
(e) the occupancy of the transcription factor or genomic complex component at a target site is altered (e.g., decreased) when the fusion molecule is present relative to when it is absent.

30. A method of delivering a fusion molecule to a cell, e.g., a mammalian cell, comprising:
providing a fusion molecule of any of claims 1-17, and
contacting the cell with the fusion molecule,
thereby delivering the fusion molecule to the cell.

31. The fusion molecule, method, cell, or reaction mixture of any preceding claim, wherein the cell is selected from a neuronal cell (e.g., a CNS cell), a myocyte (e.g., a cardiomyocyte), a blood cell (e.g., an immune cell), an endothelial cell, a hepatocyte, a CD34+ cell, a CD3+ cell, or a fibroblast.
